(19) United States
(12) Patent Application Publication
(10) Pub. No.: US 2016/0032047 A1
(43) Pub. Date: Feb. 4, 2016

(54) Peg-based adhesive phenylic derivatives and methods of synthesis and use

(71) Applicants: John L. MURPHY, (US); Jeffrey L. DALSIN, (US); Arinne N. LYMAN, (US); Christopher P. RADANO, (US); DSM IP ASSETS B.V., Heerlen (NL)

(72) Inventors: John L MURPHY, Madison, WI (US); Jeffrey L DALSIN, Verona, WI (US); Arinne N LYMAN, Exton, PA (US); Christopher P RADANO, West Chester, PA (US)

(21) Appl. No.: 14/776,187

(22) PCT Filed: Mar. 14, 2014
(86) PCT No.: PCT/US14/00056

§ 371 Date: Sep. 14, 2015

Related U.S. Application Data
(60) Provisional application No. 61/782,795, filed on Mar. 14, 2013.

(51) Int. Cl. C08G 65/48; C08L 71/02; A61L 27/18; C09D 5/16; A61L 24/04

(52) U.S. Cl. CPC C08G 65/48; C09D 5/1637; A61L 24/046; A61L 27/18; C08L 71/02; A61L 2420/06; A61L 2420/08

(57) ABSTRACT

The invention provides compositions that use phenylic derivatives to provide adhesive properties. Selection of phenylic derivatives with linkers or linking groups, and the linkages between the linkers or linking groups with polyalkylene oxides, provided herein may be configured to control curing time, biodegradation and/or swelling.

This project was funded in part by NIH (2R44DK080547-02 and 2R44DK083199-02). ¹H NMR was performed at National Magnetic Resonance Facility at Madison, Wis., which is supported by NIH (2R44DK080547-02 and 2R44DK083199-02), the University of Wisconsin, and the USDA. The government has certain rights in the invention.

### FIELD OF THE INVENTION

The invention relates generally to medical adhesives with components often found in plant life, and their structural analogues, to adhere to biologic and synthetic surfaces. Modification of polymers with these components allows for cohesive and adhesive crosslinking under oxidative conditions.

### BACKGROUND OF THE INVENTION

Phenolic derivatives such as catechol and guaiacol derivatives are naturally occurring compounds found in nature. Catechol moieties may be associated with mussel adhesive proteins (MAPs) that use this derivative to form tenacious bonds in aqueous solutions. Alternatively, guaiacol derivatives are often associated with plants, and form the structural components of lignins. These structural components are formed through the oxidative crosslinking of the phenolic group to form polymer chains. This oxidative process also forms covalent bonds between amines and thiols on tissue surfaces. While various phenylic derivatives may be used to create an adhesive of use in, for example, surgical applications, guaiacol derivatives including, for example, ferulic acid and hydroferulic acid, may have advantages over other adhesive moieties. For example, ferulic acid is an abundant and widespread cinnamic acid derivative found in its free and bound form, and may be polymerized through oxidative processes. In vivo, ferulic acid may be coupled to polysaccharides through ester bonds and may be oxidized to form dehydrodimers and other oligomeric structures to form the structural components in plant cell walls. Moreover, ferulic acid may have metal-chelating properties as well as cytoprotective-properties as a result of antioxidant activity. Accordingly, ferulic acid is a useful and safe compound when used as an adhesive moiety in, for example, surgical applications.

Phenolic compounds which allow incorporation of oxidants may be used as medical adhesives. In turn, phenolic oxidative adhesive properties may be found in compounds that are not phenolic in nature with, for example, adhesive components that contain a phenyl derivative with at least one hydroxyl, thiol, or amine. In certain embodiments of the present invention, there may be at least one additional functional group on the phenyl ring adjacent to the hydroxyl, thiol, or amine. In some embodiments, a functional group on the molecule allows attachment to polymers. Suitable functional groups for attachment to polymers include, but are not limited to, amines, thiols, hydroxyl and carboxylic acid derivatives.

In medical practice, few adhesives provide both robust adhesion in a wet environment and suitable mechanical properties to be used as a tissue adhesive or sealant. For example, fibrin-based tissue sealants (e.g., Tisseel V H, Baxter Healthcare) provide a mechanical match for natural tissue, but possess poor tissue-adhesion characteristics. Conversely, cyanoacrylate adhesives (e.g., Dermabond, Ethicon, Inc.) produce adhesive bonds with tissue surfaces, but may be stiff and brittle with regard to mechanical properties and thus not match mechanical properties of tissue. Furthermore, cyanoacrylate adhesives release formaldehyde (associated with cytotoxicity) as they degrade. Therefore, a need exists for materials that overcome one or more of the current disadvantages.

### BRIEF SUMMARY OF THE INVENTION

1. A compound comprising formula (I):

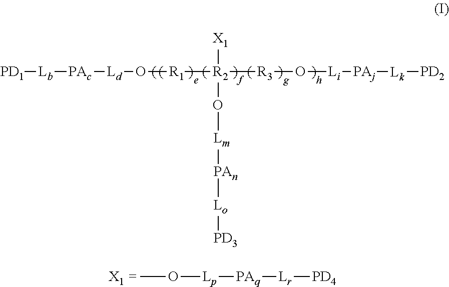

wherein

X₁ is optional;
each PD₁, PD₂, PD₃, and PD₄, independently, can be the same or different;

each L_{b}, Lₖ, Lₒ and Lᵣ, independently, can be the same or different;

optionally, each L_{d}, Lᵢ, Lₘ and Lₚ, if present, can be the same or different and if not present, represent a bond between the O and respective PA of the compound;

each PA_{c}, PAⱼ and PAₙ, independently, can be the same or different;

e is a value from 1 to about 3;

f is a value from 1 to about 10;

g is a value from 1 to about 3;

h is a value from 1 to about 10;

each of R₁, R₂ and R₃, independently, is a branched or unbranched alkyl group having at least 1 carbon atom;
each PA, independently, is a substantially poly(alkylene oxide) polyether or derivative thereof;

each L, independently, is a linker or is a suitable linking group selected from amide, ether, ester, urea, carbonate or urethane linking groups; and

each PD, independently, is a phenyl derivative, wherein

each of PD₁, PD₂, PD₃, and PD₄, independently, is a residue comprising:

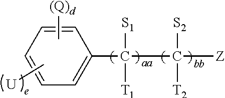

wherein Q is a OH, SH, or NH₂
“d” is 1 to 5
U is a H, OH, OCH, O-PG, SH, S-PG, NH2, NH-PG, N(PG)₂, NO₂, F, Cl, Br, or I, or combination thereof;
“e” is 1 to 5
“d+e” is equal to 5
each T₁, independently, is H, NH₂, OH, or COOH;
each S₁, independently, is H, NH₂, OH, or COOH;
each T₂, independently, is H, NH₂, OH, or COOH;
each S₂, independently, is H, NH₂, OH, or COOH;
Z is COOH, NH₂, OH or SH;
aa is a value of 0 to about 4;
bb is a value of 0 to about 4; and
optionally, when one of the combinations of T₁ and T₂, S₁ and S₂, T₁ and S₂ or S₁ and T₂ are absent, then a double bond is formed between Cₐₐ and C_{bb}, and aa and bb are each at least 1 to form the double bond when present.

In one aspect of formula (I), X₁ is not present, each PD₁, PD₂, and PD₃ are carboxylic acid containing phenylic derivatives, L_{b}, Lₖ, and Lₒ are amide linkages, each of L_{d}, Lᵢ, and Lₘ represent ether bonds, each of PA_{c}, PAⱼ, and PAₙ are polyethylene glycol polyether derivatives each comprising an amine terminal residue that forms amide linkages between the acid residue of the phenylic derivative and the polyethylene glycol polyether derivative, each having a molecular weight of between about 1,500 and about 3,500 daltons, wherein e, f and g each a value of 1, each R₁ and R₃ is a CH₂ and R₂ is a CH; and h is 6.

In yet another aspect of formula (I), X₁ is not present, each of the linkers, L_{b}, Lₖ, and Lₒ, form an amide linkage between the acid residue of the phenylic derivative and the terminal amine of an amino acid residue and an ester between the carboxylic acid portion of the amino acid residue and the terminal portion of the polyethylene glycol polyether; each of L_{d}, Lᵢ and Lₘ represent ether bonds; each of PA_{c}, PAⱼ and PAₙ are polyethylene glycol polyether derivatives comprising a hydroxyl terminal residue, each having a molecular weight of between about 1,500 and about 3,500 daltons; wherein e, f and g each have a value of 1; each R₁ and R₃ is a CH₂ and R₂ is a CH; and h is 6. In particular L_{b}, Lₖ, and Lₒ can be, glycine, B-alanine, alanine, gamma-aminobutyric acid, 3-aminobutanoic acid, 3-amino-4-methylpentanoic acid, 2-methyl-beta-alanine, 5-Aminovaleric acid, 6-Aminohexanoic acid, 7-aminoheptanoic acid, 8-aminooctanoic acid, 11-Aminoundecanoic acid, isoleucine, leucine, methionine, phenylalanine, proline, tryptophan, valine, asparagines, cysteine, glutamine, serine, threonine, tyrosine, aspartic acid, glutaric acid, arginine, hystidine, lysine, cyclohexylalanine, allylglycine, vinylglycine, proparglyglycine, norvaline, norleucine, phenylglycine, citrulline, homoserine, hydroxyproline, diaminobutanoic acid, diaminopropionic acid, or ornithine residues.

These and other embodiments of the invention described throughout the specification may be used for wound closure, and materials of this type are often referred to as tissue sealants or surgical adhesives.

In some embodiments, compounds of the present invention may be applied to a suitable substrate surface as a film or coating. Application of the compound(s) to the surface inhibits or reduces the growth of biofilm (bacteria) on the surface relative to an untreated substrate surface. In other embodiments, the compounds of the invention may be employed as an adhesive.

Exemplary applications include, but are not limited to, fixation of synthetic (resorbable and non-resorbable) and biological membranes and meshes for hernia repair, void-eliminating adhesive for reduction of post-surgical seroma formation in general and cosmetic surgeries, fixation of synthetic (resorbable and non-resorbable) and biological membranes and meshes for tendon and ligament repair, sealing incisions after ophthalmic surgery, sealing of venous catheter access sites, bacterial barrier for percutaneous devices, as a contraceptive device, a bacterial barrier and/or drug depot for oral surgeries (e.g. tooth extraction, tonsillectomy, cleft palate, etc.), for articular cartilage repair, for antifouling or anti-bacterial adhesion.

In some embodiments, reaction products of the syntheses described herein are included as compounds or compositions useful as adhesives or surface treatment/antifouling aids. It should be understood that the reaction product(s) of the synthetic reactions may be purified by methods known in the art, such as diafiltration, chromatography, recrystallization/precipitation and the like or may be used without further purification.

It should be understood that the compounds of the present invention may be coated multiple times to form bi, tri, etc. layers. The layers may be of compounds of the invention per se, or of blends of a compound(s) and polymer, or combinations of a compound layer and a blend layer, etc. Consequently, constructs may also include such layering of the compounds per se, blends thereof, and/or combinations of layers of a compound(s) per se and a blend or blends.

While multiple embodiments are disclosed, further embodiments of the present invention will become apparent to those skilled in the art from the following detailed description.

As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 shows the structure of Surphys-059

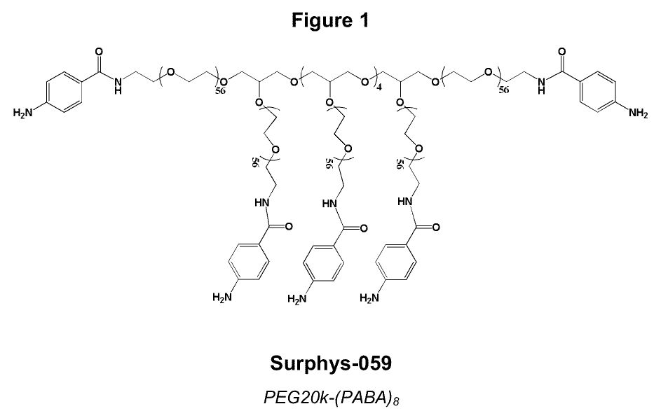

FIG. 2 shows the structure of Surphys-061

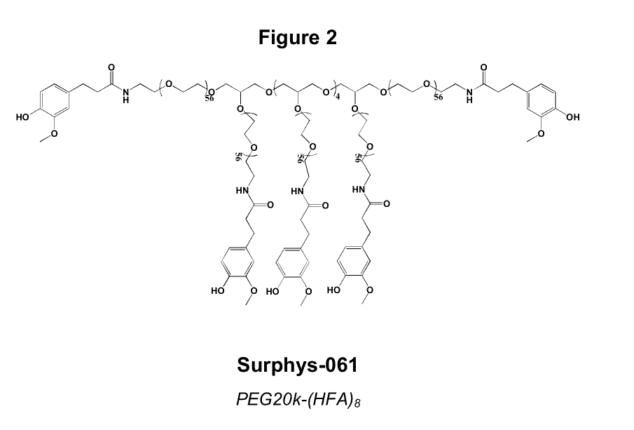

FIG. 3 shows the structure of Surphys-062

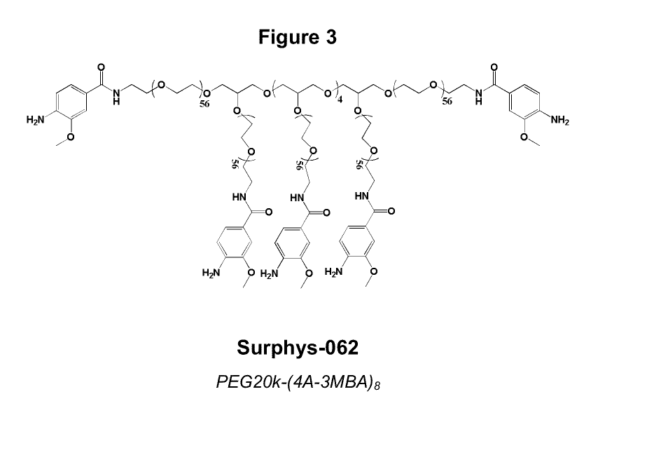

FIG. 4 shows the structure of Surphys-068

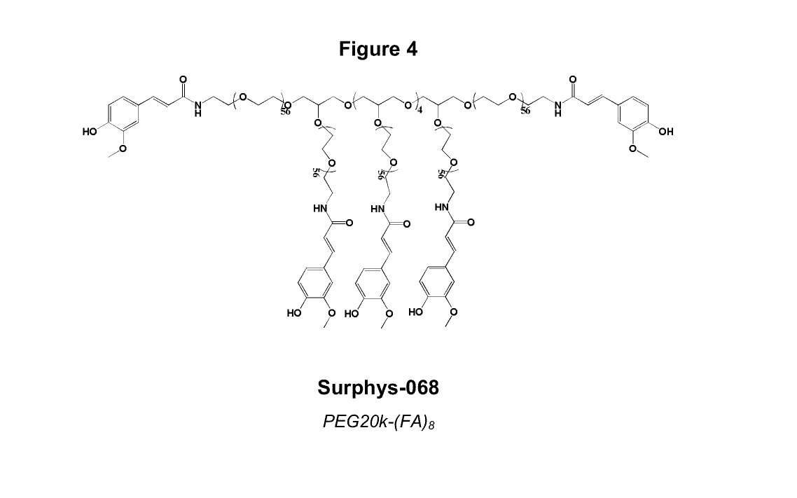

FIG. 5 shows the structure of Surphys-069

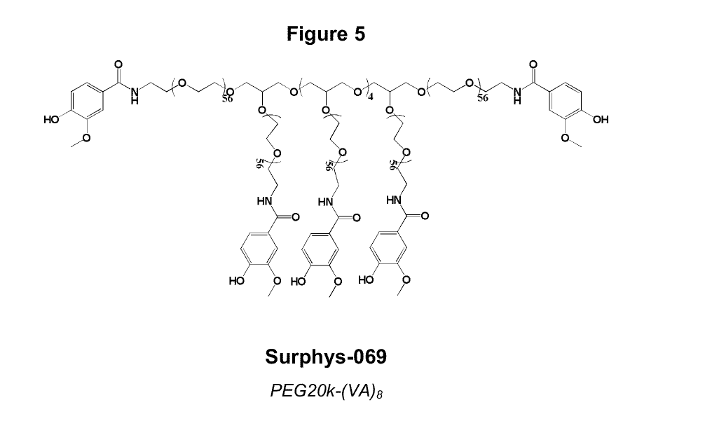

FIG. 6 shows the structure of Surphys-077

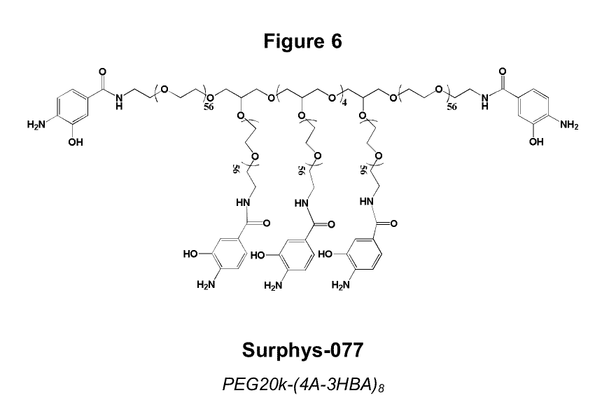

FIG. 7 shows the structure of Surphys-079

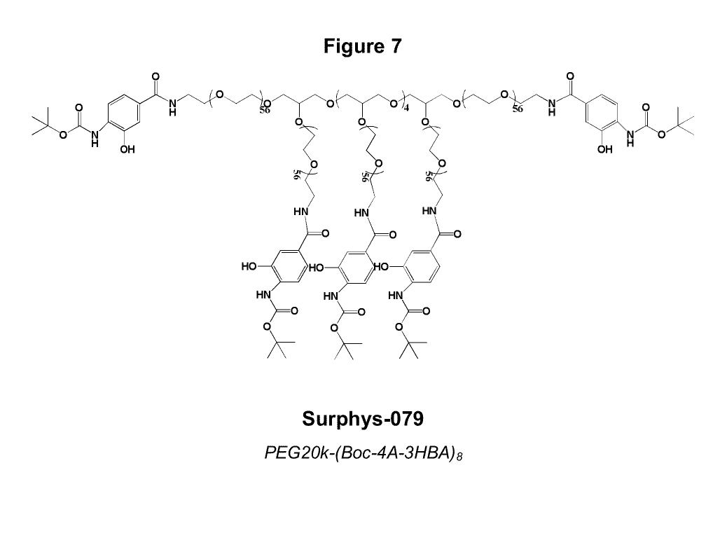

FIG. 8 shows the structure of Surphys-081

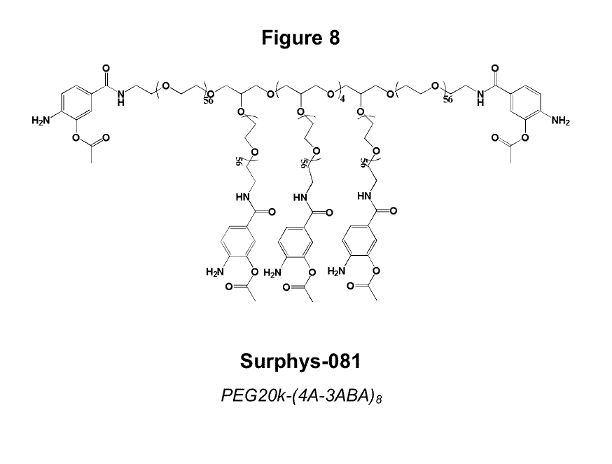

FIG. 9 shows the structure of Surphys-083

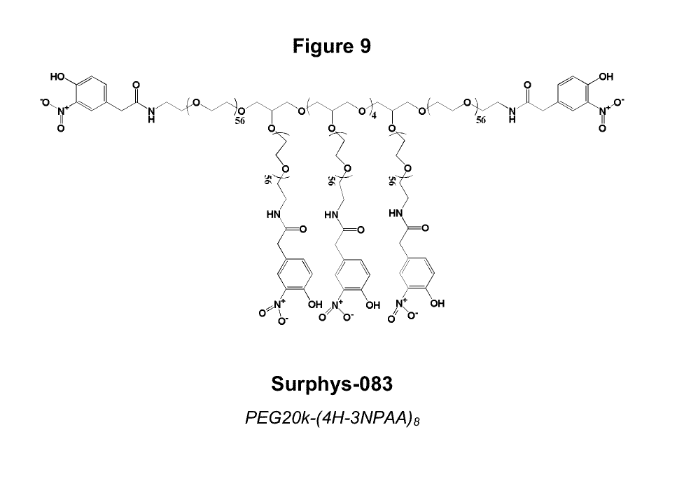

FIG. 10 shows the structure of Surphys-085

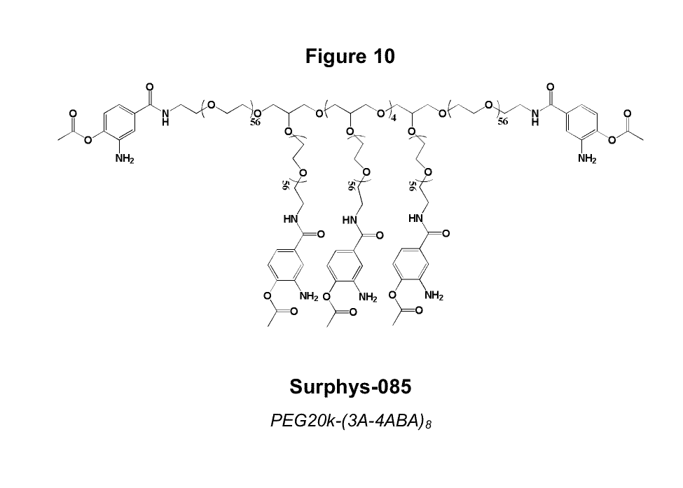

FIG. 11 shows the structure of Surphys-087

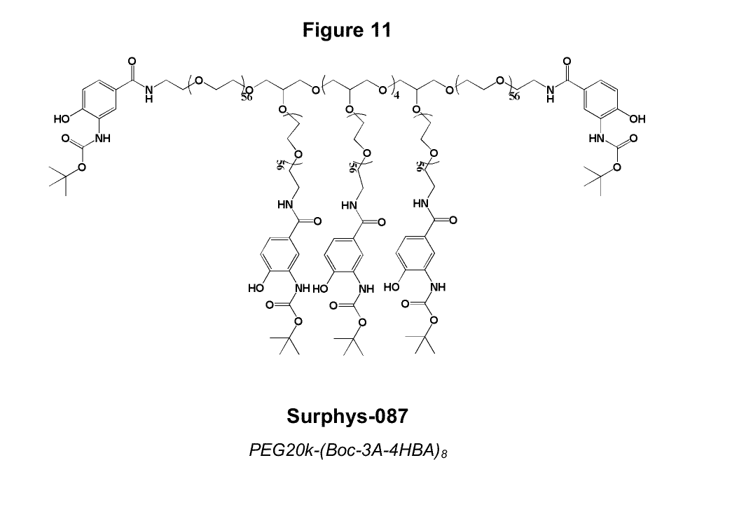

FIG. 12 shows the structure of Surphys-089

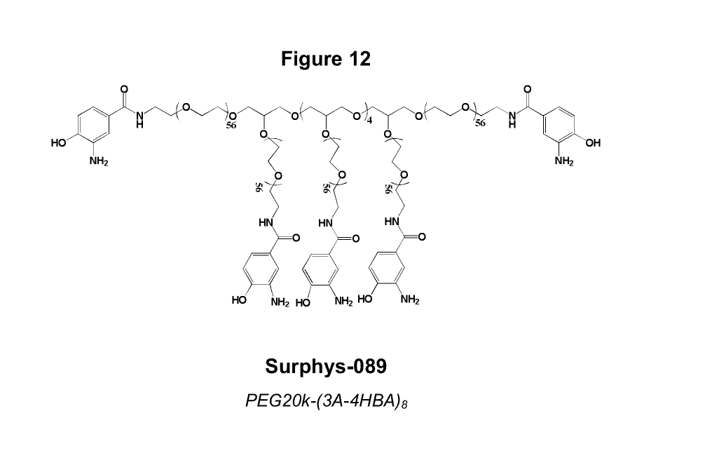

FIG. 13 shows the structure of Medhesive-077

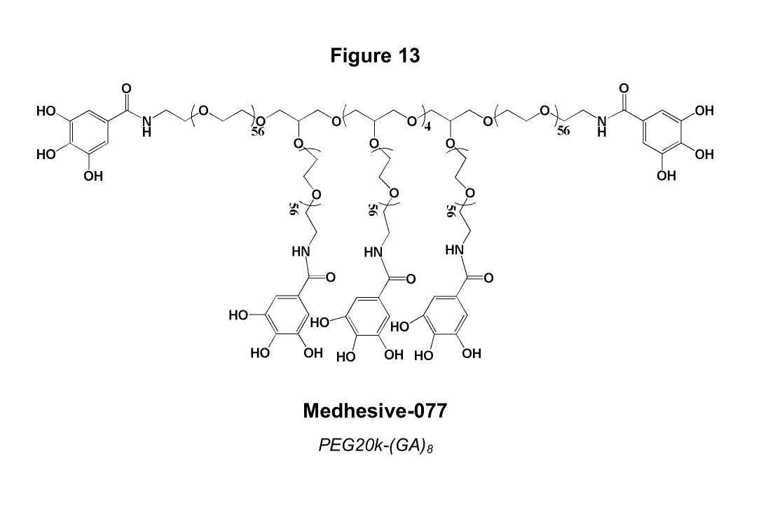

FIG. 14 shows the structure of Medhesive-079

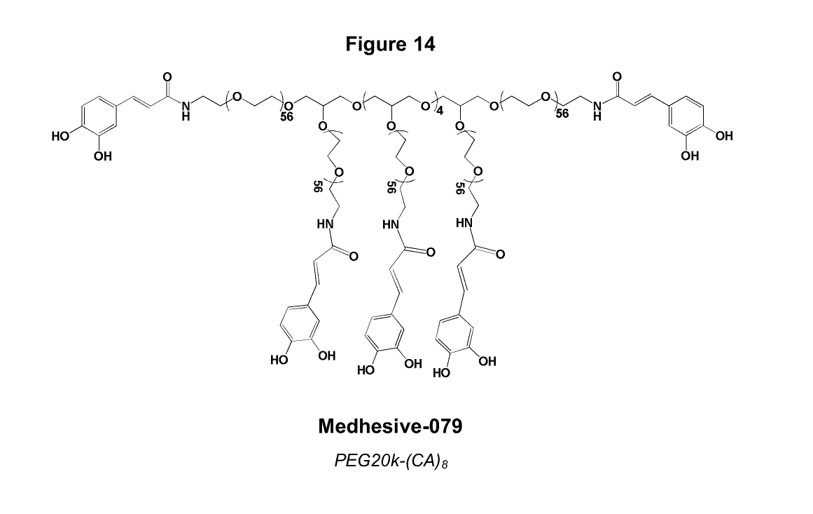

FIG. 15 shows the structure of Medhesive-117

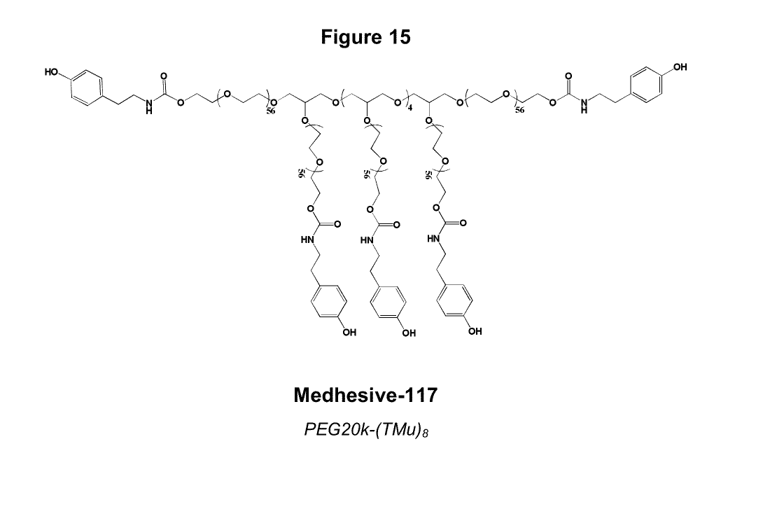

FIG. 16 shows the structure of Medhesive-120

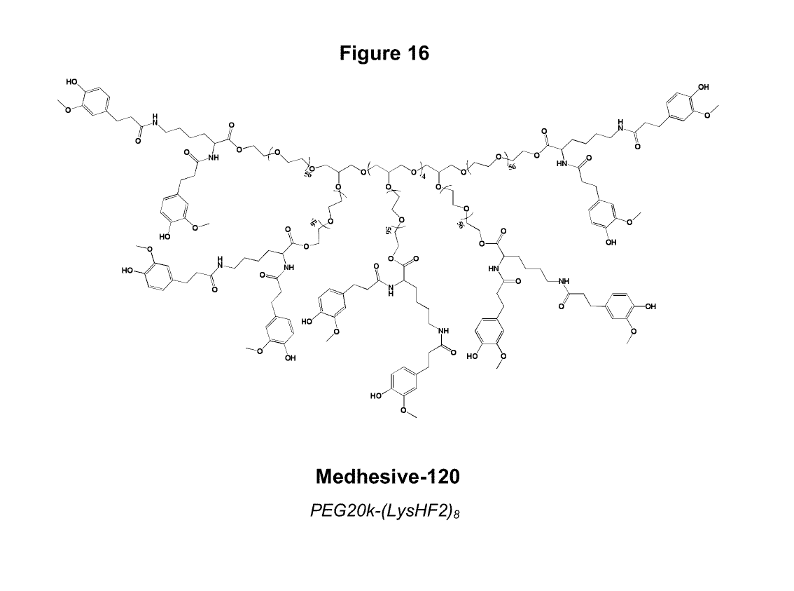

FIG. 17 shows the structure of Medhesive-121

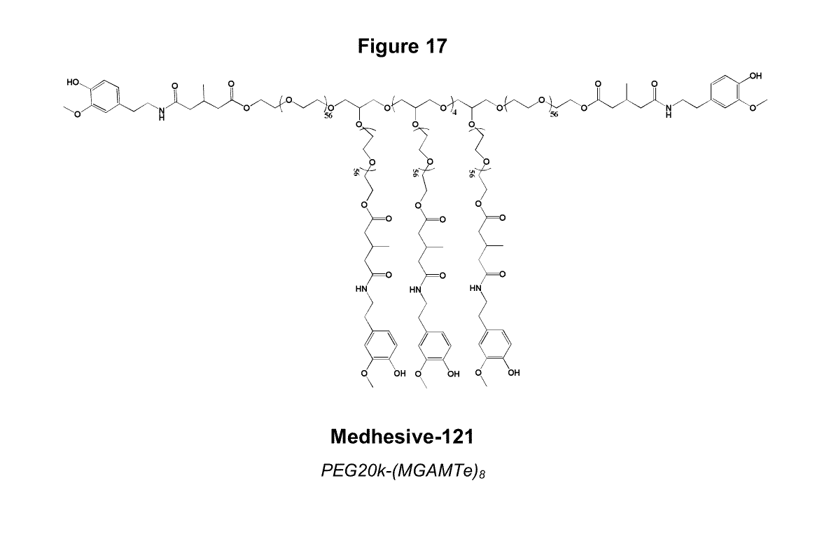

FIG. 18 shows the structure of Medhesive-122

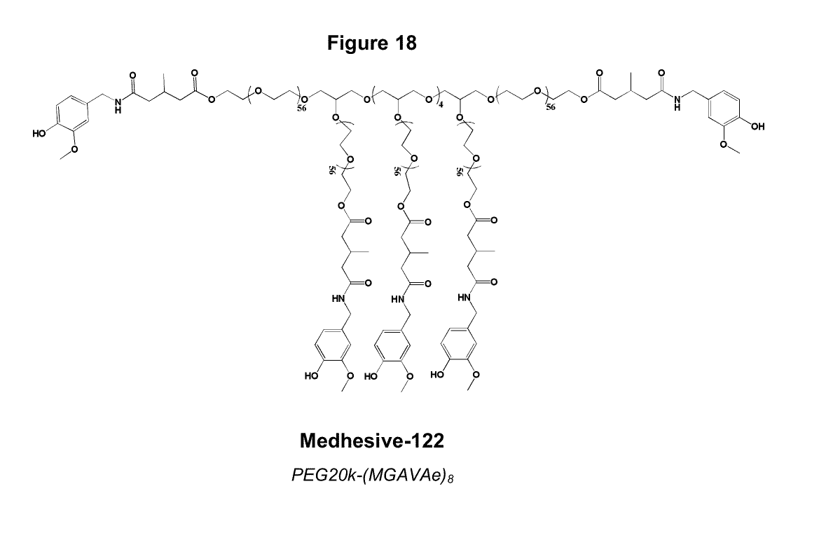

FIG. 19 shows the structure of Medhesive-123

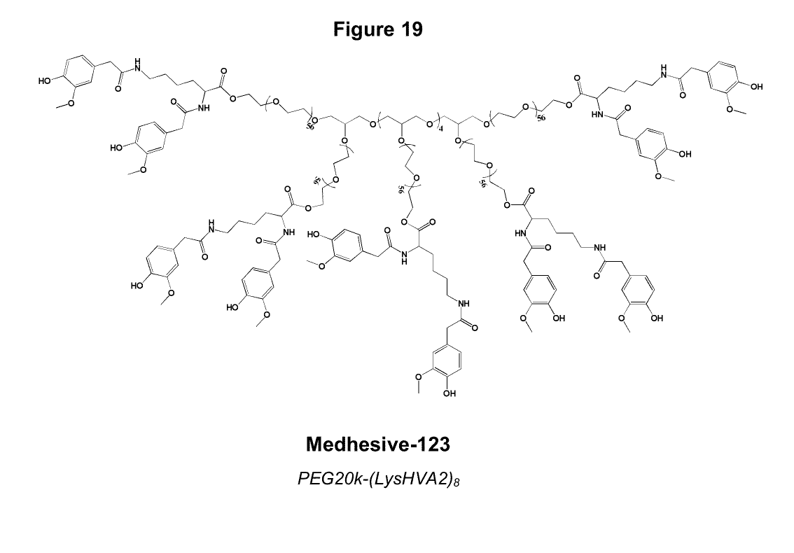

FIG. 20 shows the structure of Medhesive-125

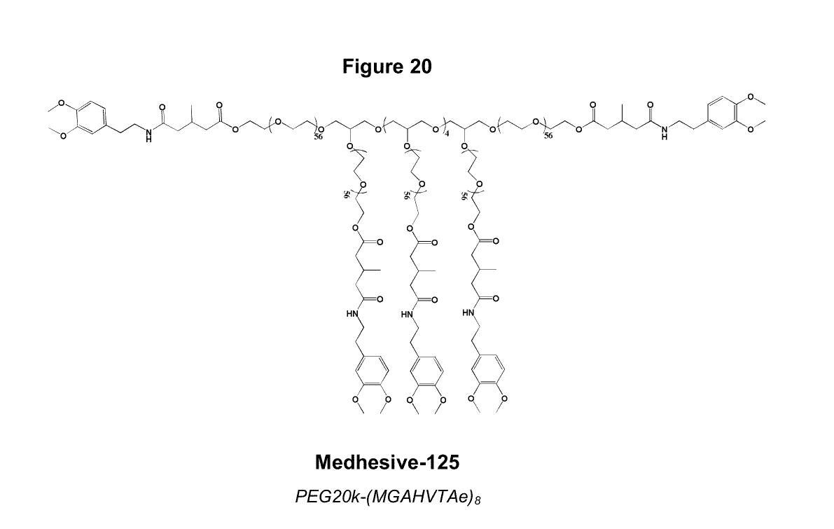

FIG. 21 shows the structure of Medhesive-126

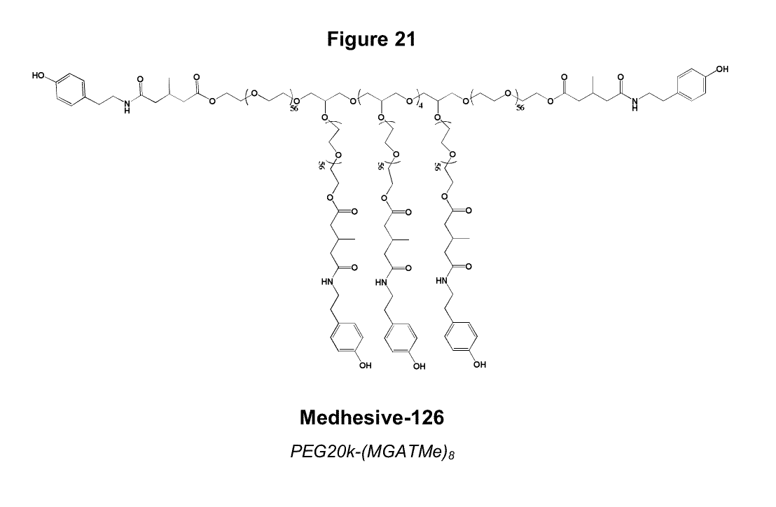

FIG. 22 shows the structure of Medhesive-127

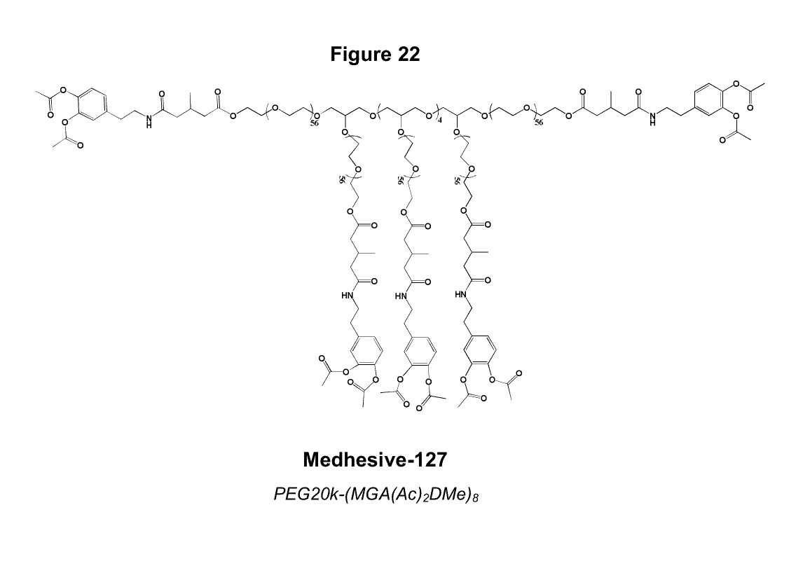

FIG. 23 shows the structure of Medhesive-128

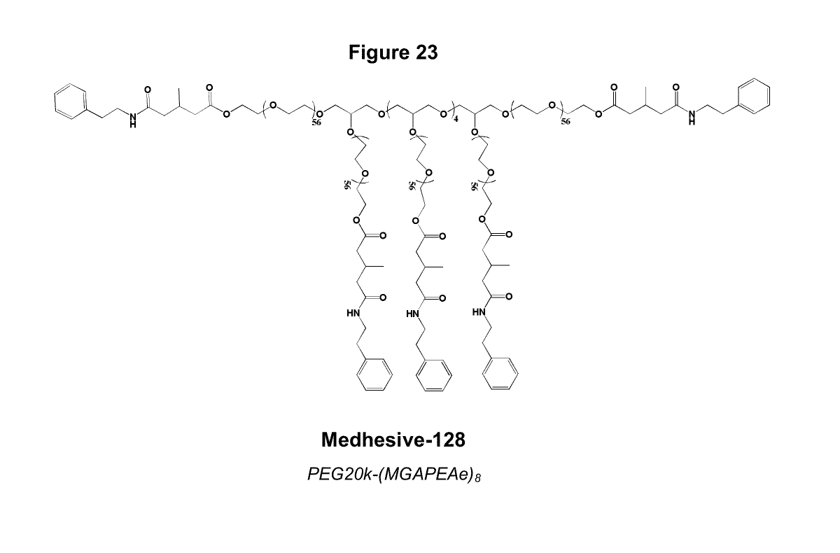

FIG. 24 shows the structure of Medhesive-129

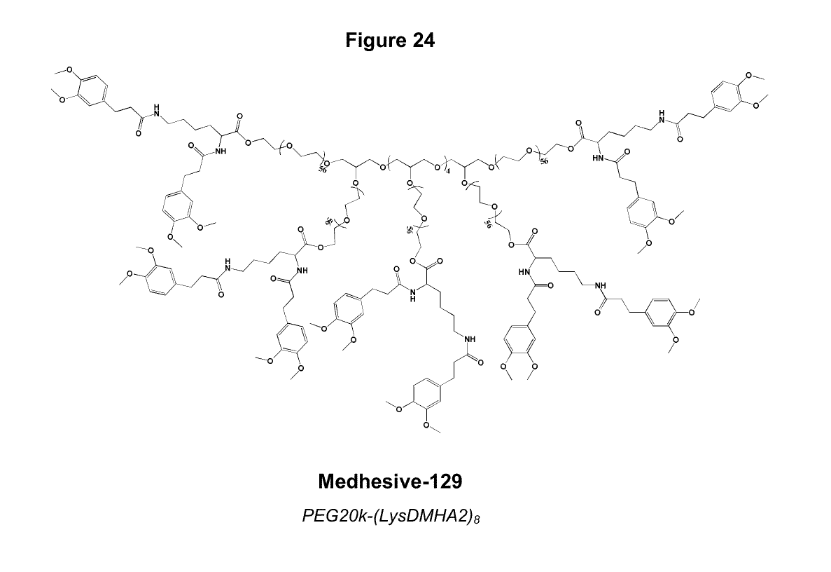

FIG. 25 shows the structure of Medhesive-130

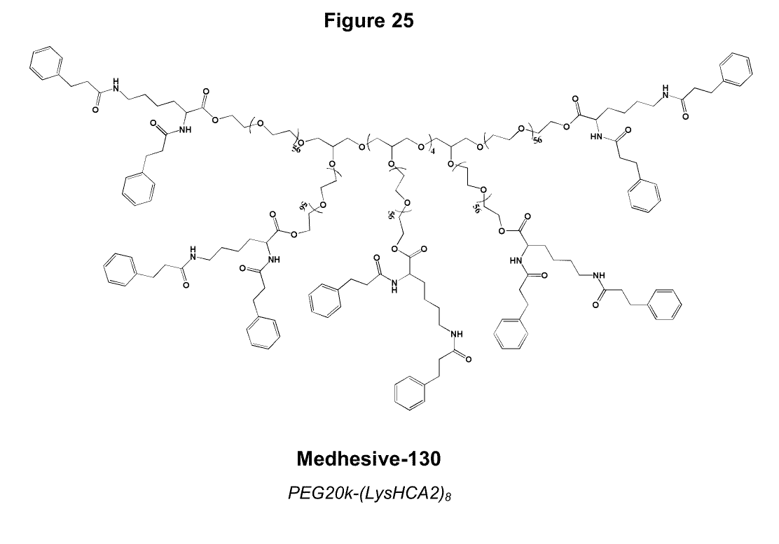

FIG. 26 shows the structure of Medhesive-134

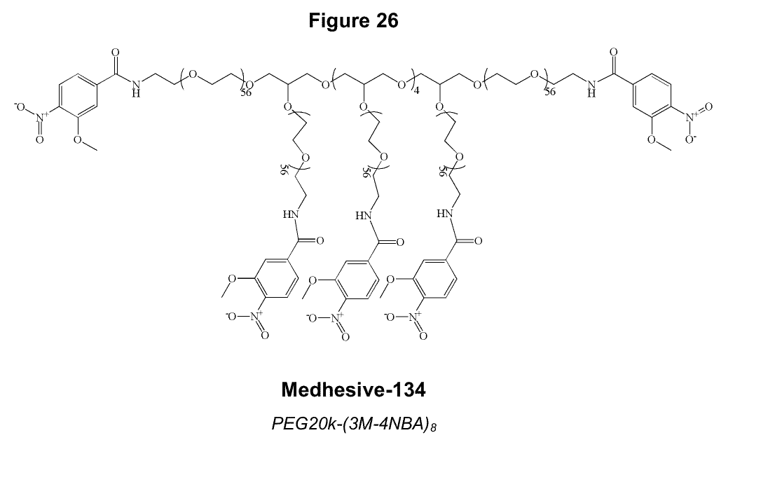

FIG. 27 shows the structure of Medhesive-135

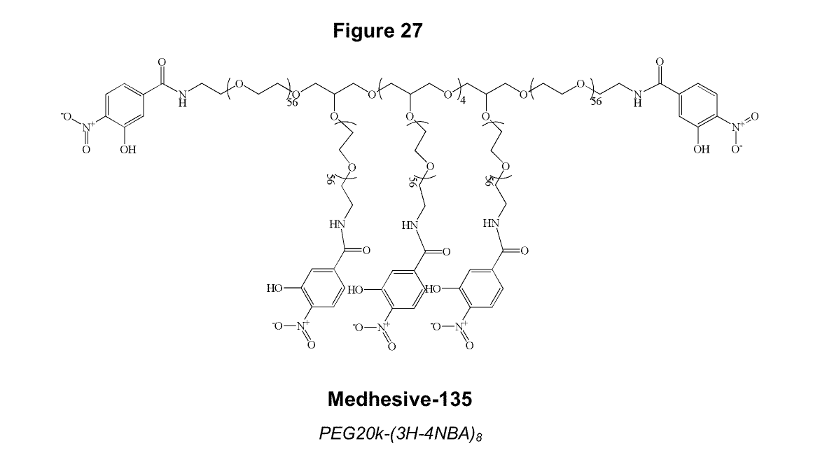

FIG. 28 shows the structure of Medhesive-155

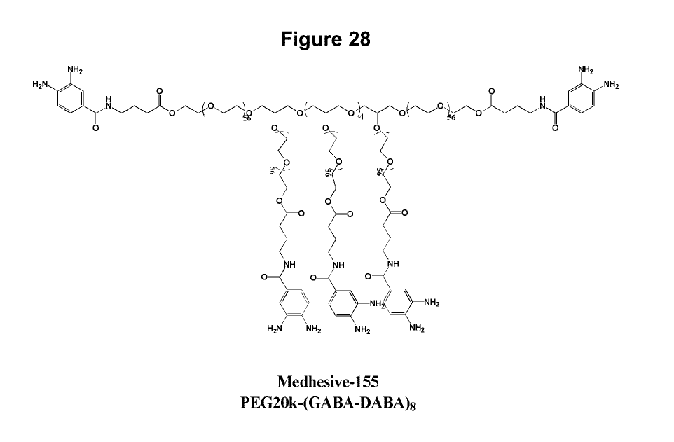

FIG. 29 shows the structure of Medhesive-160

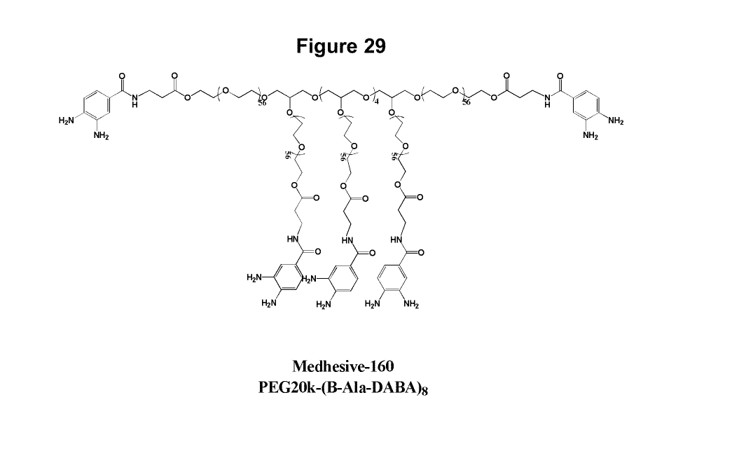

FIG. 30 shows the structure of Medhesive-161

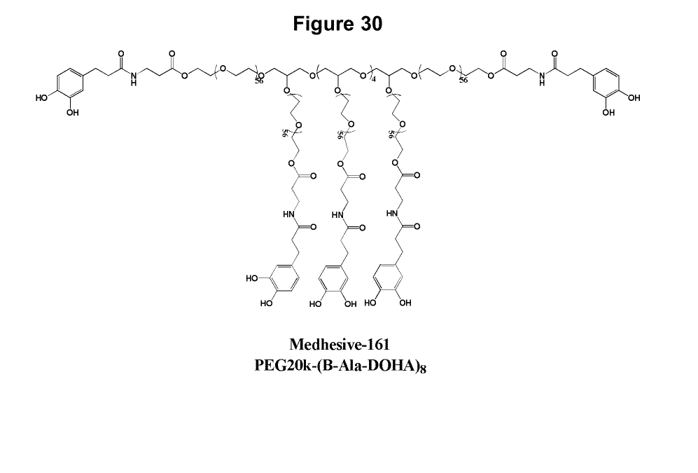

FIG. 31 shows the structure of Medhesive-149

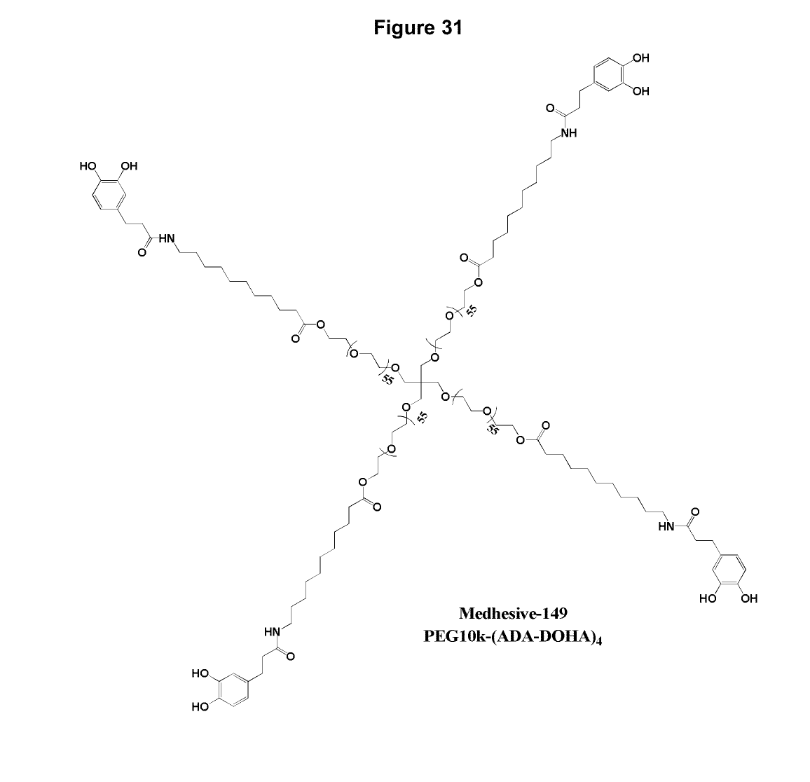

FIG. 32 shows gel permeation chromatography (GPC) plots illustrating crosslink functionality of dihydroxyphenyl-PEG5k-OCH₃ (Surphys-074) and diaminophenyl-PEG5k-OCH₃ (Surphys-066).

FIG. 33 shows the spray pattern of Medhesive-102, Medhesive-069, Medhesive-155, Medhesive-160, and Medhesive-161 at 900 on collagen.

FIG. 34 shows the structure of Medhesive-233

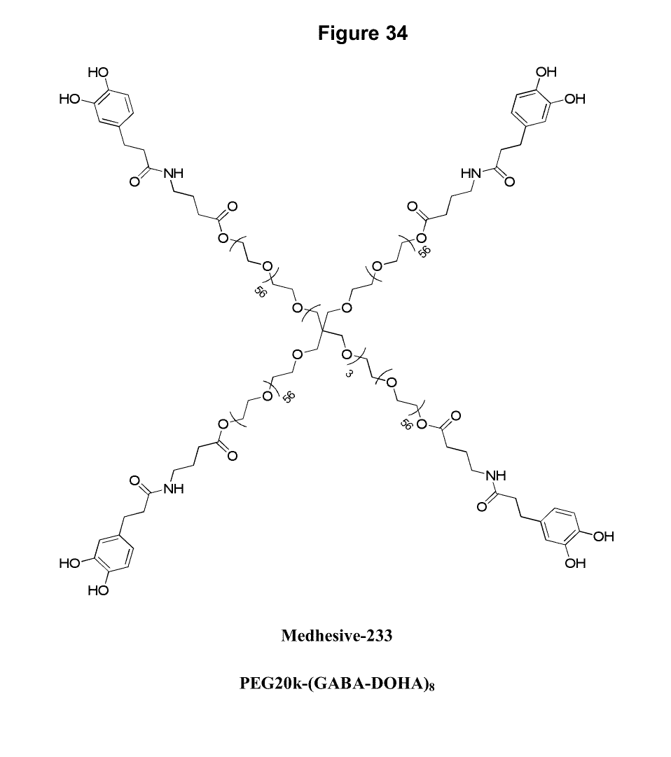

FIG. 35 shows the structure of Medhesive-228

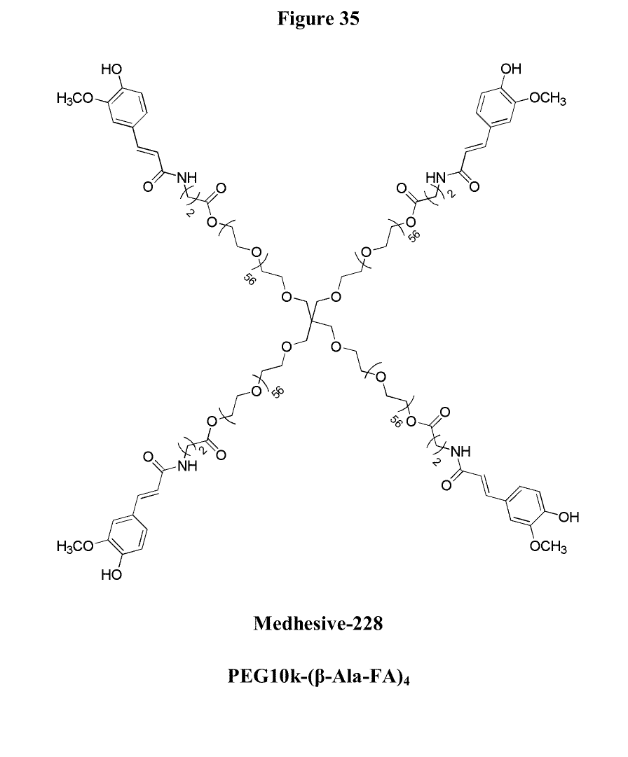

FIG. 36 shows the structure of Medhesive-229

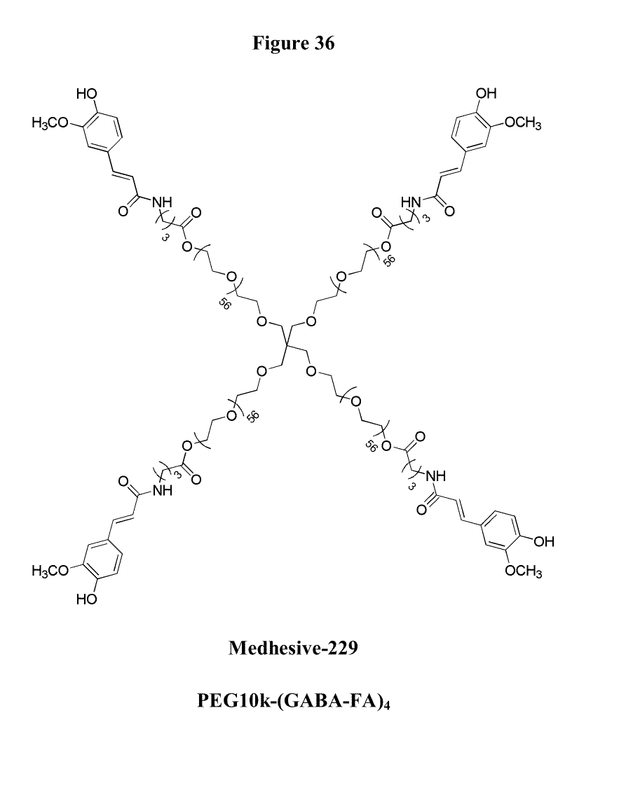

FIG. 37 shows the structure of Medhesive-230

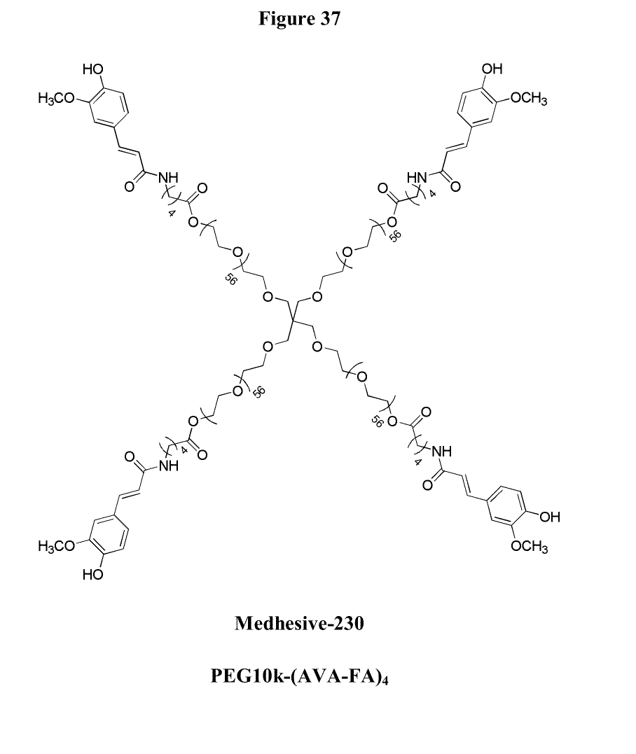

FIG. 38 shows the structure of Medhesive-235

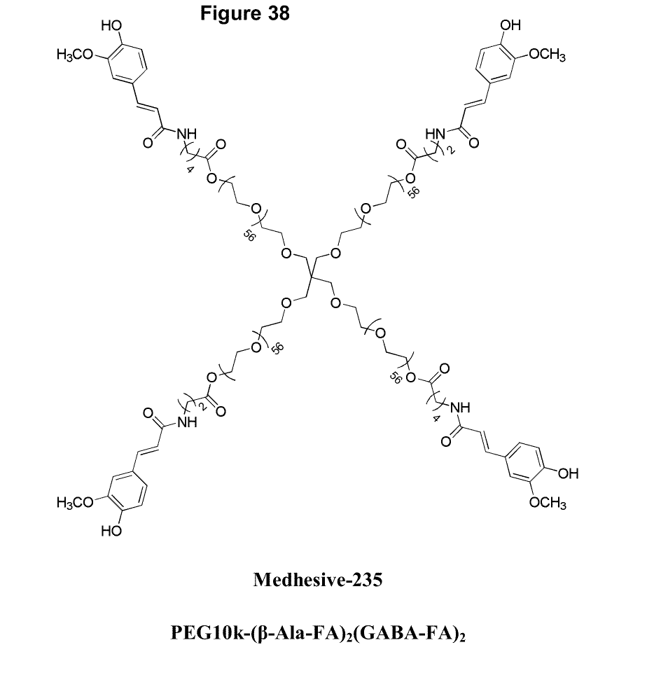

FIG. 39 is a graph of the degradation profiles of certain polymers according to the invention

### DETAILED DESCRIPTION

In the specification and in the claims, the terms “including” and “comprising” are open-ended terms and should be interpreted to mean “including, but not limited to . . . . ” These terms encompass the more restrictive terms “consisting essentially of” and “consisting of.” It must be noted that as used herein and in the appended claims, the singular forms “a”, “an”, and “the” include plural reference unless the context clearly dictates otherwise. As well, the terms “a” (or “an”), “one or more” and “at least one” may be used interchangeably herein. It is also to be noted that the terms “comprising”, “including”, “characterized by” and “having” may be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl(allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 15 carbon atoms (C₁-C₁₅ alkyl), more preferably from 1 to 10 carbon atoms (C₁-C₁₀ alkyl) and even more preferably from 1 to 6 carbon atoms (C₁-C₆ alkyl or lower alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkyldiyl” by itself or as part of another substituent refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center may form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In preferred embodiments, the alkyldiyl group comprises from 1 to 6 carbon atoms (C1-C6 alkyldiyl). Also preferred are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno,” by itself or as part of another substituent, refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkyleno group is (C1-C6) or (C1-C3) alkyleno. Also preferred are straight-chain saturated alkano groups, e.g., methano, ethano, propano, butano, and the like.

“Alkylene” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkylene is indicated in square brackets. Typical alkylene groups include, but are not limited to, methylene (methano); ethylenes such as ethano, etheno, ethyno; propylenes such as propano, prop[1]eno, propa[1,2]dieno, prop[1]yno, etc.; butylenes such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkylene group is (C1-C6) or (C1-C3) alkylene. Also preferred are straight-chain saturated alkano groups, e.g., methano, ethano, propano, butano, and the like.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂O—, —S(O)₂OR^{b}, —OS(O)₂R^{b}, —OS(O)₂O—, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O—, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each Re is independently R^{b} or alternatively, the two R^{c}s are taken together with the nitrogen atom to which they are bonded form a 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O—, —S(O)₂OR^{b}, —OS(O)₂R^{b}, —OS(O)₂O—, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O—, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O—, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and Re are as previously defined.

Substituent groups useful for substituting nitrogen atoms in heteroalkyl and cycloheteroalkyl groups include, but are not limited to, —R^{a}, —O—, —OR^{b}, —SR^{b}, —S—, —NR^{C}R^{c}, trihalomethyl, —CF₃, —CN, —NO, —NO₂, —S(O)₂R^{b}, —S(O)₂O—, —S(O)₂OR^{b}, —OS(O)₂R^{b}, —OS(O)₂O—, —OS(O)₂OR^{b}, —P(O)(O—)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups from the above lists useful for substituting other specified groups or atoms will be apparent to those of skill in the art.

The substituents used to substitute a specified group may be further substituted, typically with one or more of the same or different groups selected from the various groups specified above.

Protecting Group (PG), when used, is to represent the protecting of a hydroxyl, thiol, or amine with a group that protects it from side reactions during a synthetic procedure. In some embodiments, incorporation of a Protecting Group in an adhesive prevents oxidation of the adhesive prior to its use, for example, during storage prior to implantation of the adhesive in the body of a living being. In particular embodiments, the adhesive component with incorporation of a PG comprises an activating agent or initiator in the adhesive formulation. For instance, it is well known that amines may be protected with Boc or Fmoc, while hydroxyl and thiols may be protected with acetyl groups. In some embodiments of the present invention, Boc protecting groups consist of the reaction products between a primary amine and, for example, a di-tert-butyl dicarbonate. While di-tert-butyl dicarbonate may be used to generate Boc protected amines, alternative methods of synthesis may use leaving groups such as chlorine or NHS with the Boc protecting group in other embodiments. A result is formation of a urethane linkage between a Boc protecting group and a primary amine. The protecting group may be cleaved with acids such as concentrated HCl or trifluoroacetic acid, among others. In further embodiments, a Fmoc protecting group, for example, 9-Fluorenylmethyl chloroformate, reacts with a primary amine to form a urethane linkage wherein a chlorine group is removed to form urethane. While chlorine leaving groups may couple amines and the Fmoc protecting group, other leaving groups, such as NHS or anhydrides of FMOC may be used in other embodiments. The result is a Fmoc protected amine which may be removed with a base, for example, piperidine.

In other embodiments, other PG are used. For example, in some embodiments, a protecting group may encompass a substituted or unsubstituted, branched or unbranched, hydrocarbon as a protecting group for hydroxyl, thiol, or amine groups. In certain embodiments, a hydrocarbon group may be placed onto a hydroxyl or thiol group. In further embodiments, a hydrocarbon may be attached to an amine to form a secondary or tertiary amine or a quaternary ammonium ion.

The identifier “PA” refers to a poly(alkylene oxide) or substantially poly(alkylene oxide) and means predominantly or mostly alkyloxide or alkyl ether in composition. This definition contemplates the presence of heteroatoms e.g., N, O, S, P, etc. and of functional groups e.g., —COOH, —NH₂, —SH, or —OH as well as ethylenic or vinylic unsaturation. It is to be understood any such non-alkyleneoxide structures will only be present in such relative abundance as not to materially reduce, for example, the overall surfactant, non-toxicity, or immune response characteristics, as appropriate, of this polymer. It should also be understood that PAs may include terminal end groups such as PA-O—CH₂—CH₂—NH₂, e.g., PEG-O—CH₂—CH₂—NH₂ (as a common form of amine terminated PA). PA-O—CH₂—CH₂—CH₂—NH₂, e.g., PEG-O—CH₂—CH₂—CH₂—NH₂ is also available as well as PA-O—(CH₂—CH(CH₃)—O)ₓₓ—CH₂—CH(CH₃)—NH₂, where xx is 0 to about 3, e.g., PEG-O—(CH₂—CH(CH₃)—O)ₓₓ—CH₂—CH(CH₃)—NH₂ and a PA with an acid end-group typically has a structure of PA-O—CH₂—COOH, e.g., PEG-O—CH₂—COOH or PA-O—CH₂—CH₂—COOH, e.g., PEG-O—CH₂—CH₂—COOH. These may be considered “derivatives” of the PA. These are all contemplated as being within the scope of the invention and should not be considered limiting.

Generally each PA of the molecule has a molecular weight between about 1,250 and about 5,000 daltons and most particularly between about 1,500 and about 3,500 daltons. Therefore, it should be understood that the desired MW of the whole or combined polymer is between about 5,000 and about 50,000 Da, in particular a MW of between about 10,000 and about 20,000 Da, where the molecule has 3 to eight “arms”, each arm having a MW of between about 1,250 and about 5,000 daltons, and in particular a MW of 1,500 and about 3,500 Da, e.g., about 3300 daltons, or about 2,500 daltons.

Suitable PAs (polyalkylene oxides) include polyethylene oxides (PEOs), polypropylene oxides (PPOs), polyethylene glycols (PEGs) and combinations thereof that are commercially available from SunBio Corporation, JenKem Technology USA, NOF America Corporation or Creative PEGWorks. In one embodiment, the PA is a polyalkylene glycol polyether or derivative thereof, and most particularly is polyethylene glycol (PEG), the PEG unit (arm) having a molecular weight generally in the range of between about 1,250 and about 12,500 daltons, in particular between about 2,500 and about 10,000 daltons, e.g., 5,000 daltons. It should be understood that, for example, polyethylene oxide may be produced by ring opening polymerization of ethylene oxide as is known in the art.

In one embodiment, the PA may be a block copolymer of a PEO and PPO or a PEG or a triblock copolymer of PEO/PPO/PEO.

It should be understood that the PA terminal end groups may be functionalized. Typically the end groups are OH, NH₂, COOH, or SH. However, these groups may be converted into a halide (Cl, Br, I), an activated leaving group, such as a tosylate or mesylate, an ester, an acyl halide, N-succinimidyl carbonate, 4-nitrophenyl carbonate, and chloroformate with the leaving group being N-hydroxy succinimide, 4-nitrophenol, and Cl, respectively, etc.

The notations of “L”, “FnL” and “L” refer, respectively, to a linker, functional linker and a linking group.

A “linker” (L) refers to a moiety that has two points of attachment on either end of the moiety. For example, an alkyl dicarboxylic acid HOOC-alkyl-COOH (e.g., succinic acid) would “link” a terminal end group of a PA (such as a hydroxyl or an amine to form an ester or an amide respectively) with a reactive group of the PD (such as an NH₂, OH, or COOH). Suitable linkers include an acyclic hydrocarbon bridge (e.g., a saturated or unsaturated alkyleno such as methano, ethano, etheno, propano, prop[1]eno, butano, but[1]eno, but[2]eno, buta[1,3]dieno, and the like), a monocyclic or polycyclic hydrocarbon bridge (e.g., [1,2]benzeno, [2,3]naphthaleno, and the like), a monocyclic or polycyclic heteroaryl bridge (e.g., [3,4]furano[2,3]furano, pyridino, thiopheno, piperidino, piperazino, pyrazidino, pyrrolidino, and the like) or combinations of such bridges, dicarbonyl alkylenes, etc. Suitable dicarbonyl alkylenes include, C2 through C10 dicarbonyl alkylenes such as malonic acid, succinic acid, 3-methylglutaric acid, glutaric acid, etc. Additionally, the anhydrides, acid halides and esters of such materials may be used to effect the linking when appropriate.

Other suitable linkers include moieties that have two different functional groups that may react and link with an end group of a PA. These include groups such as amino acids (glycine, lysine, aspartic acid, etc.), amino acid derivatives (β-alanine, γ-aminobutyric acid, 11-aminoundecaoic acid, etc.) and moieties such as dopamine. For example, an amine protected β-alanine derivative may be attached to PEG through normal ester coupling reactions to form an ester linkage between the PEG polymer backbone and the carboxylic acid of the amine protected β-alanine. The amine protecting group may be removed through normal deprotection chemistry of amines to form a primary amine. This primary amine may react with a PD derivative through normal peptide coupling chemistries to form an amide bond.

A functional linker (FnL) is a linker, such as those noted above, that includes one or more moieties that can react with a reactive site of the PD molecule. Generally such moieties are amines, esters, carboxylic acids, etc. For example, aspartic is a dicarboxylic acid with an amine group. The dicarboxylic acid portion of the molecule may be reacted to form part of the polymer backbone while the amine portion can be reacted with the PD, forming, for example, an amide bond, e.g., where the amide bond is a “L”. The functional linker can contain several moieties that can react with reactive sites of PD molecules. For example, lysine, is a diamine with a carboxylic acid residue. Consequently, condensation of lysine with PD molecules and a PEG provide a molecule that contains two amide bonds, where the PD's contain reactive esters, and an ester where the terminal carboxylic acid/ester forms the ester bond with the hydroxyl of a PEG. This can be signified by PD-L-FnL-(L-PD)-L, where the FnL contains three points of attachment to the polymer backbone (amide, amide, ester).

It should be understood that two or more linkers may be adjacent to each other. In such embodiments, two reactive portions of the two or more linkers combine to form a bond, such as an ester bond, an amide bond, etc. (L). For example, a carboxylic acid can react with a group that includes a hyroxyl group, such that an ester is formed. Many combinations can be envisaged between various linkers and are contemplated within the scope of this application. Additionally, the one or more of the linkers can be functional linkers.

A linking group (L) refers to the reaction product of the terminal end moieties of the PA and PD (the situation where “b” is 0; no linker present) condense to form an amide, ether, ester, urea, carbonate or urethane linkage depending on the reactive sites on the PA and PD. In other words, a direct bond is formed between the PA and PD portion of the molecule and no linker is present.

The term “residue” is used to mean that a portion of a first molecule reacts (e.g., condenses) with a portion of a second molecule to form, for example, a linking group, such as an amide, ether, ester, urea, carbonate or urethane linkage depending on the reactive sites on the PA and PD.

The denotation “PD” refers to a phenyl derivative, which contains a functional group “Z” that can be reacted with amines, thiols, hydroxyls and/or acidic groups on a polymer backbone. The phenyl group contains at least one functional group (Q) chosen from a hydroxyl (—OH), thiol (—SH), or an amine (—NH₂) group. A second functional group (Q or U) chosen from H, OH, OOCCH₃, NH₂, NH-Boc, NH-Fmoc, NH(CH₃), N(CH₃)₂, OCH₃, NO₂, F, Cl, Br, or I. As an example of a suitable PD, a ferulic acid derivative. Suitable PD derivatives include the formula:

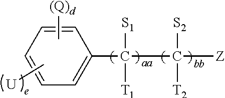

### Wherein: Q is a OH, SH, or NH₂;

“d” is 1 to 5;
U is a H, OH, OCH₃, O-PG, SH, S-PG, NH2, NH-PG, N(PG)₂, NO₂, F, Cl, Br, or I, or combination thereof;
“e” is 1 to 5;
“d+e” is equal to 5;
each T₁, independently, is H, NH₂, OH, or COOH;
each S₁, independently, is H, NH₂, OH, or COOH;
each T₂, independently, is H, NH₂, OH, or COOH;
each S₂, independently, is H, NH₂, OH, or COOH;

### Z is COOH, NH₂, OH or SH;

aa is a value of 0 to about 4;
bb is a value of 0 to about 4; and

Optionally, when one of the combinations of T₁ and T₂, S₁ and S₂, T₁ and S₂ or S₁ and T₂ are absent, then a double bond is formed between Cₐₐ and C_{bb}, and aa and bb are each at least 1, to form the double bond when present.

In one embodiment, each S₁, S₂, T₁ and T₂ are hydrogen atoms, aa is 1, bb is 1 and Z is either COOH or NH₂.

In another embodiment, S₁ and S₂ are both hydrogen atoms, T₁ and T₂ are not present, aa is 1, bb is 1, and Z is COOH or NH₂.

In still another embodiment, S₁ and T₁ are both hydrogen atoms, aa is 1, bb is 0, and Z is COOH or NH₂.

In still another embodiment, aa is 0, bb is 0 and Z is COOH or NH₂.

It should be understood that where aa is 0 or bb is 0, then S₁ and T₁ or S₂ and T₂, respectively, are not present.

It should be understood, that upon condensation of the PD molecule with the PA that a molecule of water, for example, is generated such that a bond is formed as described above (i.e., an amide, ether, ester, urea, carbonate or urethane bond).

In particular, PD molecules include, but are not limited to, dopamine, 3,4-dihydroxy phenylalanine (DOPA), 3,4-dihydroxyhydrocinnamic acid, 3,4-dihydroxyphenyl ethanol, 3,4 dihydroxyphenylacetic acid, 3,4 dihydroxyphenylamine, 3,4-dihydroxybenzoic acid, gallic acid, 2,3,4, trihydroxybenzoic acid and 3,4 dihydroxycinnamic acid, caffeic acid, ferulic acid, isoferulic acid, vanillic acid, hydroferulic acid, homovanillic acid, 3-methoxytyramine, tyramine, vanillylamine, sinapic acid, syringic acid, coumaric acid, 4-hydroxybenzoic acid, 3-hydroxybenzoic acid, 3,4-diaminobenzoic acid, 3-amino-4-hydroxybenzoic acid, 4-amino-3-hydroxybenzoic acid, Boc-3-amino-4-hydroxybenzoic acid, Boc-4-amino-3-hydroxybenzoic acid, 3-amino-4-acetoxybenzoic acid, 4-amino-3-acetoxybenzoic acid, 4-mercaptobenzoic acid, 4-aminobenzoic acid, 3-aminobenzoic acid, 4-amino-3-methoxybenzoic acid, 3-amino-4-methoxybenzoic acid, 4-hydroxy-3-nitrobenzoic acid, 3-hydroxy-4-nitrobenzoic acid, 4-hydroxy-3-nitrophenylacetic acid, 3-hydroxy-4-nitrophenylacetic acid, 4-amino-3-nitrobenzoic acid, 3-amino-4-nitrobenzoic acid, 3-fluoro-4-hydroxybenzoic acid, 4-fluoro-3-hydroxybenzoic acid, 3-chloro-4-hydroxybenzoic acid, 3,5-dichloro-4-hydroxybenzoic acid, 4-chloro-3-hydroxybenzoic acid, 3-bromo-4-hydroxybenzoic acid, 4-bromo-3-hydroxybenzoic acid, 4-hydroxy-3-iodobenzoic acid, 3-hydroxy-4-iodobenzoic acid, 4-amino-3-iodonezoic acid, 3-amino-4-iodobenzoic acid, 3-fluoro-4-aminobenzoic acid, 4-fluoro-3-aminobenzoic acid, 3-chloro-4-aminobenzoic acid, 3,5-dichloro-4-aminobenzoic acid, 4-chloro-3-aminobenzoic acid, 3-bromo-4-aminobenzoic acid, 4-bromo-3-aminobenzoic acid, 3-fluoro-4-hydroxyphenylacetic acid, 4-fluoro-3-hydroxyphenylacetic acid, 3-chloro-4-hydroxyphenylacetic acid, 4-chloro-3-hydroxyphenylacetic acid, 3-bromo-4-hydroxyphenylacetic acid, 4-bromo-3-hydroxyphenylacetic acid, 3-hydroxy-4-iodophenylacetic acid, 4-hydroxy-3-iodophenylacetic acid

In some embodiments, the present invention provides a multi-armed, poly (alkylene oxide) polyether, phenyl derivative (PD) having the general formula:

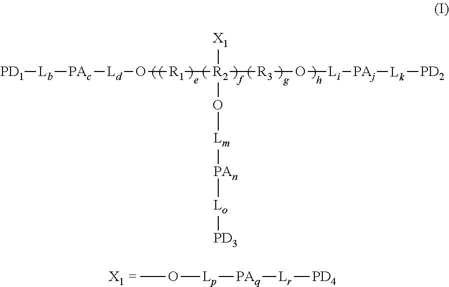

wherein

X₁ is optional;

each PD₁, PD₂, PD₃, and PD₄, independently, can be the same or different;

each L_{b}, Lₖ, Lₒ and Lᵣ, independently, can be the same or different;

optionally, each L_{d}, Lᵢ, Lₘ and Lₚ, if present, can be the same or different and if not present, represent a bond between the O and respective PA of the compound;

each PA_{c}, PAⱼ and PAₙ, independently, can be the same or different;

e is a value from 1 to about 3;

f is a value from 1 to about 10;

g is a value from 1 to about 3;

h is a value from 1 to about 10;

each of R₁, R₂ and R₃, independently, is a branched or unbranched alkyl group having at least 1 carbon atom;

each PA, independently, is a substantially poly(alkylene oxide) polyether or derivative thereof;

each L, independently, is a linker or is a suitable linking group selected from amide, ether, ester, urea, carbonate or urethane linking groups; and

each PD, independently, is a phenyl derivative.

each of PD₁, PD₂, PD₃, and PD₄, independently, is a residue of a formula comprising:

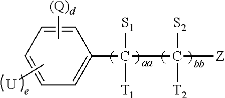

Wherein: Q is a OH, SH, or NH₂;
“d” is 1 to 5;
U is a H, OH, OCH₃, O-PG, SH, S-PG, NH2, NH-PG, N(PG)₂, NO₂, F, Cl, Br, or I, or combination thereof;
“e” is 1 to 5;
“d+e” is equal to 5;
each T₁, independently, is H, NH₂, OH, or COOH;
each S₁, independently, is H, NH₂, OH, or COOH;
each T₂, independently, is H, NH₂, OH, or COOH;
each S₂, independently, is H, NH₂, OH, or COOH;
Z is COOH, NH₂, OH or SH;
aa is a value of 0 to about 4;
bb is a value of 0 to about 4; and
Optionally, when one of the combinations of T₁ and T₂, S₁ and S₂, T₁ and S₂ or S₁ and T₂ are absent, then a double bond is formed between Cₐₐ and C_{bb}, and aa and bb are each at least 1 to form the double bond when present.

In one embodiment, X₁ is not present, each PD₁, PD₂, and PD₃ are carboxylic acid containing phenylic derivatives, L_{b}, Lₖ, and Lₒ are amide linkages, each of L_{d}, Lᵢ, and Lₘ represent ether bonds, each of PA_{c}, PAⱼ, and PAₙ are polyethylene glycol polyether derivatives each comprising an amine terminal residue which form the amide linkages between the acid residue of the phenylic derivative and the polyethylene glycol polyether derivative, each having a molecular weight of between about 1,500 and about 3,500 daltons, wherein e, f and g each a value of 1, each R₁ and R₃ is a CH₂ and R₂ is a CH; and h is 6.

In yet another embodiment of formula (I), each of the linkers, L_{b}, Lₖ, and Lₒ, form an amide linkage between the acid residue of the phenylic derivative and the terminal amine of an amino acid residue and an ester between the carboxylic acid portion of the amino acid residue and the terminal portion of the polyethylene glycol polyether; each of L_{d}, Lᵢ and Lₘ represent ether bonds; each of PA_{c}, PAⱼ and PAₙ are polyethylene glycol polyether derivatives comprising a hydroxyl terminal residue, each having a molecular weight of between about 1,500 and about 3,500 daltons; wherein e, f and g each a value of 1; each R₁ and R₃ is a CH₂ and R₂ is a CH; and h is 6. In particular L_{b}, Lₖ, and Lₒ can be, glycine, B-alanine, alanine, gamma-aminobutyric acid, 3-aminobutanoic acid, 3-amino-4-methylpentanoic acid, 2-methyl-beta-alanine, 5-Aminovaleric acid, 6-Aminohexanoic acid, 7-aminoheptanoic acid, 8-aminooctanoic acid, 11-Aminoundecanoic acid, isoleucine, leucine, methionine, phenylalanine, proline, tryptophan, valine, asparagines, cysteine, glutamine, serine, threonine, tyrosine, aspartic acid, glutaric acid, arginine, hystidine, lysine, cyclohexylalanine, allylglycine, vinylglycine, proparglyglycine, norvaline, norleucine, phenylglycine, citrulline, homoserine, hydroxyproline, diaminobutanoic acid, diaminopropionic acid, or omithine residues.

It should be understood that where ranges are provided, such as where “f” for example has a value of from 1 to about 10, that every value between is contemplated by the applicant and is included herein for all purposes. Therefore, every value can be relied upon to provide novel and inventive compositions and their uses.

In one embodiment, X₁ of formula (I) is not present, each of PD₁, PD₂, and PD₃ of is a phenyl derivative residue, each of L_{b}, Lₖ, and Lₒ are amide linkages, each of L_{d}, Lᵢ and Lₘ represent bonds, each of PA_{c}, PAⱼ and PAₙ are polyethylene glycol polyether derivatives each comprising an amine terminal residue which form the amide linkages between the acid residue of the PD and the polyethylene glycol polyether derivative, each having a molecular weight of between about 1,500 and about 3,500 daltons, wherein e, f and g each a value of 1, each R₁ and R₃ is a CH₂ and R₂ is a CH; and h is 6.

In another embodiment of formula (I), X₁ is not present, each of PD₁, PD₂, and PD₃, is a phenyl derivative residue; each of L_{b}, Lₖ, and Lₒ are urethane linkages between the amine residue of the PD and the terminal portion of the polyethylene glycol polyether; each of L_{d}, Lᵢ and Lₘ represent bonds; each of PA_{c}, PAⱼ and PAₙ are polyethylene glycol polyether derivatives comprising a hydroxyl terminal residue which form the urethane linkage between the amine residue and the polyethylene glycol polyether derivative, each having a molecular weight of between about 1,500 and about 5,000 daltons; wherein e, f and g each a value of 1; each R₁ and R₃ is a CH₂ and R₂ is a CH; and h is 6.

In yet another embodiment of formula (I), X₁ is not present, each of PD₁, PD₂, and PD₃ is a PD containing an amine residue; each of the linkers, L_{b}, Lₖ, and Lₒ, form an amide linkage between the PD amine residue and one terminal portion of a dicarboxylic acid residue and an ester between the second terminal portion of the dicarboxylic acid residue and the terminal portion of the polyethylene glycol polyether; each of L_{d}, Lᵢ and Lₘ represent bonds; each of PA_{c}, PAⱼ and PAₙ are polyethylene glycol polyether derivatives comprising a hydroxyl terminal residue, each having a molecular weight of between about 1,500 and about 3,500 daltons; wherein e, f and g each a value of 1; each R₁ and R₃ is a CH₂ and R₂ is a CH; and h is 6.

In yet another embodiment of formula (I), X₁ is not present, each of PD₁, PD₂, and PD₃ is a PD containing a carboxylic acid residue; each of the linkers, L_{b}, Lₖ, and Lₒ, form an amide linkage between the PD carboxylic acid residue and the terminal amine portion of an amino acid derivative residue and an ester between the terminal carboxylic acid portion of the amino acid derivative residue and the terminal portion of the polyethylene glycol polyether; each of L_{d}, Lᵢ and Lₘ represent bonds; each of PA_{c}, PAⱼ and PAₙ are polyethylene glycol polyether derivatives comprising a hydroxyl terminal residue, each having a molecular weight of between about 1,500 and about 3,500 daltons; wherein e, f and g each a value of 1; each R₁ and R₃ is a CH₂ and R₂ is a CH; and h is 6.

In still yet another embodiment of formula (I), X₁ is not present, each of PD₁, PD₂, and PD₃ is a PD containing a carboxylic acid residue; each of L_{b}, Lₖ, and Lₒ are amide linkages; each of L_{d}, Lᵢ and Lₘ represent bonds; each of PA_{c}, PAⱼ and PAₙ are polyethylene glycol polyether derivatives each comprising an amine terminal residue which form the amide linkages between the acid residue and the polyethylene glycol polyether derivative, each having a molecular weight of between about 1,500 and about 3,500 daltons; wherein e, g and h each have a value of 1; each R₁ and R₃ is a CH₂ and R₂ is a CH; and f is 4. The molecular weights of PA_{c}, PAⱼ and PAₙ are each about 1,500 daltons or the molecular weights of PA_{c}, PAⱼ and PAₙ are each about 2,500 daltons or the molecular weights of PA_{c}, PAⱼ and PAₙ are each about 3,300 daltons.

In one aspect, X₁ of formula (I) exists and each of PD₁, PD₂, PD₃, and PD₄ is a phenyl derivative residue, each of L_{b}, Lₖ, Lₒ, and Lᵣ are amide linkages, each of L_{d}, Lᵢ, Lₘ, and L represent bonds, each of PA_{c}, PAⱼ, PAₙ, and PA_{q} are polyethylene glycol polyether derivatives each comprising an amine terminal residue which form the amide linkages between the acid residue of the PD and the polyethylene glycol polyether derivative, each having a molecular weight of between about 1,500 and about 3,500 daltons, wherein e, f and g each a value of 1, each R₁ and R₃ is a CH₂ and R₂ is a CH₂—C—CH₂; and h is 1.

In another embodiment, X₁ of formula (I) exists and each of PD₁, PD₂, PD₃, and PD₄ is a phenyl derivative residue; each of L_{b}, Lₖ, Lₒ, and Lᵣ are urethane linkages between the amine residue of the PD and the terminal portion of the polyethylene glycol polyether; each of L_{d}, Lᵢ, Lₘ, and Lₚ represent bonds; each of PA_{c}, PAⱼ, PAₙ, and PA_{q} are polyethylene glycol polyether derivatives comprising a hydroxyl terminal residue which form the urethane linkage between the amine residue and the polyethylene glycol polyether derivative, each having a molecular weight of between about 1,500 and about 5,000 daltons; wherein e, f and g each a value of 1; each R₁ and R₃ is a CH₂ and R₂ is a CH₂—C—CH₂; and h is 1.

In yet another embodiment t, X₁ of formula (I) exists and each of PD₁, PD₂, PD₃, and PD₄ is a PD containing an amine residue; each of the linkers, L_{b}, Lₖ, Lₒ, and Lᵣ, form an amide linkage between the PD amine residue and one terminal portion of a dicarboxylic acid residue and an ester between the second terminal portion of the dicarboxylic acid residue and the terminal portion of the polyethylene glycol polyether; each of L_{d}, Lᵢ, Lₘ, and Lₚ represent bonds; each of PA_{c}, PAⱼ, PAₙ, and PA_{q} are polyethylene glycol polyether derivatives comprising a hydroxyl terminal residue, each having a molecular weight of between about 1,500 and about 3,500 daltons; wherein e, f and g each a value of 1; each R₁ and R₃ is a CH₂ and R₂ is a CH₂—C—CH₂; and h is 1.

In one embodiment, X₁ of formula (I) exists and each of PD₁, PD₂, PD₃ and PD₄ is a phenyl derivative residue, each of L_{b}, Lₖ, and Lₒ are amide linkages, each of L_{d}, Lᵢ, Lₘ, and Lₚ represent bonds, each of PA_{c}, PAⱼ and PAₙ are polyethylene glycol polyether derivatives each comprising an amine terminal residue which form the amide linkages between the acid residue of the PD and the polyethylene glycol polyether derivative, each having a molecular weight of between about 1,500 and about 3,500 daltons, wherein e, f and g each a value of 1, each R₁ and R₃ is a CH₂ and R₂ is a CH₂—C—CH₂; and h is 2.

In another embodiment, X₁ of formula (I) exists and each of PD₁, PD₂, PD₃, PD₄ is a phenyl derivative residue; each of L_{b}, Lₖ, Lₒ, and Lᵣ are urethane linkages between the amine residue of the PD and the terminal portion of the polyethylene glycol polyether; each of L_{d}, Lᵢ, Lₘ, and Lₚ represent bonds; each of PA_{c}, PAⱼ, PAₙ, and PA_{q} are polyethylene glycol polyether derivatives comprising a hydroxyl terminal residue which form the urethane linkage between the amine residue and the polyethylene glycol polyether derivative, each having a molecular weight of between about 1,500 and about 5,000 daltons; wherein e, f and g each a value of 1; each R₁ and R₃ is a CH₂ and R₂ is a CH₂—C—CH₂; and h is 2.

In yet another embodiment, X₁ of formula (I) exists and each of PD₁, PD₂, PD₃, and PD₄ is a PD containing an amine residue; each of the linkers, L_{b}, Lₖ, Lₒ, and Lᵣ form an amide linkage between the PD amine residue and one terminal portion of a dicarboxylic acid residue and an ester between the second terminal portion of the dicarboxylic acid residue and the terminal portion of the polyethylene glycol polyether; each of L_{d}, Lᵢ, Lₘ, and Lₚ represent bonds; each of PA_{c}, PAⱼ, PAₙ, and PA_{q} are polyethylene glycol polyether derivatives comprising a hydroxyl terminal residue, each having a molecular weight of between about 1,500 and about 3,500 daltons; wherein e, f and g each a value of 1; each R₁ and R₃ is a CH₂ and R₂ is a CH₂—C—CH₂; and h is 2.

In yet another embodiment, X₁ of formula (I) exists and each of PD₁, PD₂, PD₃, and PD₄ is a PD containing a carboxylic acid residue; each of the linkers, L_{b}, Lₖ, Lₒ, and Lᵣ, form an amide linkage between the PD carboxylic acid residue and the terminal amine portion of an amino acid derivative residue and an ester between the terminal carboxylic acid portion of the amino acid derivative residue and the terminal portion of the polyethylene glycol polyether; each of L_{d}, Lᵢ, Lₘ, and Lₚ represent bonds; each of PA_{c}, PAⱼ, PAₙ, PA_{q} are polyethylene glycol polyether derivatives comprising a hydroxyl terminal residue, each having a molecular weight of between about 1,500 and about 3,500 daltons; wherein e, f and g each a value of 1; each R₁ and R₃ is a CH₂ and R₂ is a CH₂—C—CH₂; and h is 2.

In one embodiment, X₁ of formula (I) exists and each of PD₁, PD₂, PD₃, and PD₄ is a phenyl derivative residue, each of L_{b}, Lₖ, Lₒ, and Lᵣ are amide linkages, each of L_{d}, Lᵢ, Lₘ, and Lₚ represent bonds, each of PA_{c}, PAⱼ, PAₙ, and PA_{q} are polyethylene glycol polyether derivatives each comprising an amine terminal residue which form the amide linkages between the acid residue of the PD and the polyethylene glycol polyether derivative, each having a molecular weight of between about 1,500 and about 3,500 daltons, wherein e, f and g each a value of 1, each R₁ and R₃ is a CH₂ and R₂ is a CH₂—C—CH₂; and h is 3.

In another embodiment, X₁ of formula (I) exists and each of PD₁, PD₂, PD₃, and PD₄ is a phenyl derivative residue; each of L_{b}, Lₖ, Lₒ, and Lₚ are urethane linkages between the amine residue of the PD and the terminal portion of the polyethylene glycol polyether; each of L_{d}, Lᵢ, Lₘ, and Lₚ represent bonds; each of PA_{c}, PAⱼ, PAₙ, and PA_{q} are polyethylene glycol polyether derivatives comprising a hydroxyl terminal residue which form the urethane linkage between the amine residue and the polyethylene glycol polyether derivative, each having a molecular weight of between about 1,500 and about 5,000 daltons; wherein e, f and g each a value of 1; each R₁ and R₃ is a CH₂ and R₂ is a CH₂—C—CH₂; and h is 3.

In yet another embodiment, X₁ of formula (I) exists and each of PD₁, PD₂, PD₃, and PD₄ is a PD containing an amine residue; each of the linkers, L_{b}, Lₖ, Lₒ, and Lᵣ, form an amide linkage between the PD amine residue and one terminal portion of a dicarboxylic acid residue and an ester between the second terminal portion of the dicarboxylic acid residue and the terminal portion of the polyethylene glycol polyether; each of L_{d}, Lᵢ, Lₘ, and Lₚ represent bonds; each of PA_{c}, PAⱼ, PAₙ, and PA_{q} are polyethylene glycol polyether derivatives comprising a hydroxyl terminal residue, each having a molecular weight of between about 1,500 and about 3,500 daltons; wherein e, f and g each a value of 1; each R₁ and R₃ is a CH₂ and R₂ is a CH₂—C—CH₂; and h is 3.

In yet another embodiment, X₁ of formula (I) exists and each of PD₁, PD₂, PD₃, and PD₄ is a PD containing a carboxylic acid residue; each of the linkers, L_{b}, Lₖ, Lₒ, and Lᵣ, form an amide linkage between the PD carboxylic acid residue and the terminal amine portion of an amino acid derivative residue and an ester between the terminal carboxylic acid portion of the amino acid derivative residue and the terminal portion of the polyethylene glycol polyether; each of L_{d}, Lᵢ, Lₘ, and Lₚ represent bonds; each of PA_{c}, PAⱼ, PAₙ, and PA_{q} are polyethylene glycol polyether derivatives comprising a hydroxyl terminal residue, each having a molecular weight of between about 1,500 and about 3,500 daltons; wherein e, f and g each a value of 1; each R₁ and R₃ is a CH₂ and R₂ is a CH₂—C—CH₂; and h is 3.

In one embodiment, a polymer consists of entirely X₁ (e.g., Surphys-066), wherein the bond connecting the O on X₁ to R₂ of formula (I) is replaced by a terminal methoxy group. In another embodiment, the bond connecting X₁ to R₂ of formula (I) is replaced by a PD residue. In either case a linear polymer with a mono- or di-substituted PD is formed. While these polymers may form adhesive hydrogels over time, there use may be limited due to a lack of central branching point in the polymer backbone. In some embodiments, it is therefore important for an adhesive hydrogel of the present invention to consist of a polymer containing at least 3 branching points.

L_{b}, Lₖ, Lₒ and Lᵣ, if present, each individually, can be a Cl to about a C18 alkyl chain that can be branched or unbranched and/or substituted with substituents such as, for example, carbonyl or amine functionalit(ies). Suitable examples include succinic acid, aminovaleric acid (AVA), 3-methylglutaric acid, glutaric acid, β-alanine, γ-aminobutyric acid, lysine or 11-aminoundecanoic acid residues. In some embodiments, the alkyl chain includes one or more heteroatoms and/or one or more degrees of unsaturation. In other embodiments, one or more of L_{b}, Lₖ, Lₒ and Lᵣ can be a bond, e.g., an amide, ether, ester, urea, carbonate, or urethane linking group.

R₁, R₂, and/or R₃, each individually when present, can be a Cl to about a C8 carbon alkyl that can be branched or unbranched and/or substituted with substituents. In some embodiments, the alkyl chain can include one or more heteroatoms and/or one or more degrees of unsaturation.

L_{d}, Lᵢ, Lₘ and Lₚ, if present, each individually, can be a C1 to about a C18 alkyl chain that can be branched or unbranched and/or substituted with substituents such as, for example, carbonyl or amine functionalit(ies). Suitable examples include succinic acid, 3-methylglutaric acid, glutaric acid, β-alanine, γ-aminobutyric acid, lysine, or 11-aminoundecanoic acid residues. Further the alkyl chain can include one or more heteroatoms and/or one or more degrees of unsaturation.

In some embodiments, one or more of L_{d}, Lᵢ, Lₘ and Lₚ can be a single bond, e.g., an amide, ether, ester, urea, carbonate, or urethane linking group.

Each PA_{c}, PAⱼ, PAₙ and PA_{q}, independently, if present, can be one of the PA's described herein.

“e” is a value from 1 to about 3.
“f” is a value from 1 to about 10.
“g” is a value from 1 to about 3.
“h” is a value from 1 to about 10.

The adhesives of the invention can be used for wound closure and materials of this type are often referred to as tissue sealants or surgical adhesives.

In some embodiments, formulations of the invention (the adhesive composition) have a solids content of between about 10% to about 50% solids by weight, in particular between about 15% and about 40% by weight and particularly between about 20% and about 35% by weight. Without wishing to be bound to a theory, it is believed that the addition of the PD, contributes to adhesive interactions on metal oxide surfaces through electrostatic interactions. Cohesion or crosslinking is achieved via oxidation of PD by sodium periodate (NaIO₄) to form reactive radical intermediates. It is further theorized, again without wishing to be bound by a theory, that these PD's can react with other nearby PD's and functional groups on surfaces, thereby achieving covalent crosslinking.

The adhesives of the invention may be used for wound closure, such as a dura sealant. In some embodiments, the adhesives of the invention are biodegradable. The biodegradation can occur via cleavage of the linking groups or linkers by hydrolysis or enzymatic means. The biodegradation can be tailored for a given application. The biodegradation preferably occurs at sites where ester linkages occur, though hydrolysis may also occur at amide and urethane linkages. In some embodiments, the degradation rate of the ester linkages may be controlled by increasing/decreasing the hydrophobicity of the linker. More hydrophobic linkers (high number of alkyl groups) may take longer to degrade than linkers which are hydrophilic (low number of alkyl groups). The degradation profile can also be tailored by the branching of the linker. Higher branched linkers will slow degradation through steric effects. The degradation products which result may be biocompatible.

In some embodiments, the biodegradation rate of the adhesive product may be tailored to a target range of use, for example, in a living being. In certain embodiments, the adhesive comprises a combination of different linkers that connect the PD and PA by one or more amide, ester, or urethane linkers, or any combination thereof. In further embodiments, the linkers comprise a mixture of dicarboxylic acids or amino acids. In some embodiments, biodegradation of a composition of the present invention is tailored by the hydrophobicity of the one or more linkers used, or by the degree of branching of the adhesive, or by both. For example, in some embodiments, a multi-armed adhesive molecule with “n” number of arms comprises at least 2 different linkers, with a first linker on 1 to (n−1) arms, and a second linker on the remaining arms. In further embodiments, 3 or more linkers (i.e., up to n) are used to provide a preferred biodegradation characteristic for the adhesive.

In another embodiment of the present invention, an adhesive comprises a blend of 2 or more structures wherein each structure comprises a single species of linkers on its arms. Such blends can comprise weight ratios of from 99:1 to 1:99 depending on desired properties of the blend. In some embodiments, the blend comprises structures with different linkers, wherein the overall hydrophobicity and degree of branching of the blend are configured to provide a preferred rate of biodegradation of an adhesive.

In yet another embodiment, an adhesive comprises a blend of at least 2 or more structures, wherein each structure comprises either identical linkers or a mixture of linkers on its arms, wherein the overall hydrophobicity and degree of branching of the blend are configured to provide a preferred rate of biodegradation of an adhesive.

In some embodiments, linkers are dicarboxylic acids or amino acids that form amide bonds from the PD to the linker, and amide or ester bonds from the linker to the PA.

As used herein, a wound includes damage to any tissue in a living organism. The tissue may be an internal tissue, such as the stomach lining, dura mater or pachymeninx or a bone, or an external tissue, such as the skin. As such a wound may include, but is not limited to, a gastrointestinal tract ulcer, a broken bone, a neoplasia, or cut or abraded skin. A wound may be in a soft tissue, such as the spleen, cardiovascular, or in a hard tissue, such as bone. The wound may have been caused by any agent, including traumatic injury, infection or surgical intervention.

As used herein, the adhesives/compositions of the invention can be considered “tissue sealants” which are substances or compositions that, upon application to a wound, seals the wound, thereby reducing blood loss and maintaining hemostasis.

Typically the adhesive composition of the invention is applied to the surface to be treated, e.g., repaired, as a formulation with a carrier (such as a pharmaceutically acceptable carrier) or as the material per se.

The phrase “pharmaceutically acceptable carrier” means a pharmaceutically-acceptable material, composition or vehicle, such as a liquid or solid filler, diluent, excipient, solvent or encapsulating material that can be combined with the adhesive compositions of the invention. Each carrier should be “acceptable” in the sense of being compatible with the other ingredients of the composition and not injurious to the individual. Some examples of materials which may serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; alginate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; phosphate buffered saline with a neutral pH, PRP (platelet-rich plasma) compositions and other non-toxic compatible substances employed in pharmaceutical formulations.

In some embodiments, the adhesive composition of the invention can be applied as a “patch” that includes any shaped substrate compatible with surgical implantation and capable of being coated by an inventive sealant. The adhesive compositions can be formulated for use as an aqueous suspension, a solution, a powder, a paste, a sheet, a ring, a stent, a cone, a plug, a pin, a screw and complex three-dimensional shapes contoured to be complementary to specific anatomical features. Inventive patch materials include collagen; polylactic acid; hyaluronic acid; alginate; fluoropolymers; silicones; knitted or woven meshes of, for example, cellulosic fibers, polyamides, rayon acetates and titanium; skin; bone; titanium and stainless steel. In some embodiments, pericardial or other body tissue may be used instead of a collagen patch. More preferably, the collagen is a flexible, fibrous sheet readily formed into a variety of shapes that is bioabsorbable and has a thickness of 1-5 millimeters. Such fibrous sheet collagen is commercially available from a number of suppliers. A collagen patch serves to enhance sealant strength while allowing some penetration of the inventive tissue sealant thereto. In some embodiments, in a surgical setting, a dry or a wetted absorbent gauze is placed proximal to the wound site in order to wick away any excess inventive tissue sealant prior to cure.

In some embodiments, the inventive tissue adhesive composition can be delivered in conjunction with a propellant that is provided in fluid communication with a spray nozzle tip. Propellants include aerosol propellants such as carbon dioxide, nitrogen, propane, fluorocarbons, dimethyl ether, hydro chloro fluoro carbon-22, 1-chloro-1,1-difluoroethane, 1,1-difluoroethane, and 1,1,1-trifluoro-2-fluoroethane, alone or in combination.

In certain embodiments an oxidant is included with the bioadhesive film layer. The oxidant can be incorporated into the polymer film or it can be contacted to the film at a later time. A solution could be sprayed or brushed onto either the adhesive surface or the tissue substrate surface. Alternatively, the construct can be dipped or submerged in a solution of oxidant prior to contacting the tissue substrate. In some embodiments, the oxidant upon activation can help promote crosslinking of the multihydroxy phenyl groups with each other and/or tissue. Suitable oxidants include periodates, NalO₃, NalO₄, alkylammonium-periodate derivatives, Ag(I) salts, (Ag(NO₃), Fe III salts, (FeCl₃), Mn III salts (MnCl₃), H₂O₂, oxygen, an inorganic base, an organic base or an enzymatic oxidase and the like.

In some embodiments, the invention further provides crosslinked bioadhesive constructs or hydrogels derived from the compositions described herein. For example, two PD moieties from two separate polymer chains can be reacted to form a bond between the two PD moieties. In some embodiments, this is an oxidative/radical initiated crosslinking reaction wherein oxidants/initiators such as one or more of the oxidants described previously may be used. In some embodiments, a ratio of oxidant/initiator to PD containing material is between about 0.1 to about 5.0 (on a molar basis) (oxidant:PD). In one particular embodiment, the ratio is between about 0.25 to about 2.0 and more particularly between about 0.5 to about 1.0. In some embodiments, periodate is effective in the preparation of crosslinked hydrogels of the invention. In some embodiments, oxidation “activates” the PD(s) which allow it to form interfacial crosslinking with appropriate surfaces with functional groups (i.e., biological tissues with —NH₂, —SH, etc.).

In some embodiments, the PD containing material is put into a first aqueous solution having a pH between about 3 and about 10, e.g., a pH of about 7-8, with a saline content of between about 0.9 to about 1.8 percent on a weight basis. FD&C Blue No. 1 can be added in a concentration range of between about 0.005 and about 0.5 percent on a weight basis, in particular between about 0.005 and about 0.02, more particularly about 0.1 weight percent. The concentration of the polymer (PD containing material) can be between about 3 to about 60 percent on a weight basis, in particular between about 10 and about 50 percent and particularly about 15 weight percent.

In some embodiments, a second solution is prepared prior to combining with the first solution. The second solution is an aqueous solution that contains between about 1 to about 50 milligrams (mg) of sodium periodate (NaIO₄) per ml of solution, in particular between about 4 and about 25 mg/ml and particularly between about 7-14.

In some embodiments, when the PD containing material is treated with an oxidant/initiator as described, the material sets (crosslinks) within 100 seconds, more particularly within 30 seconds, even more particularly 5 seconds, most particularly under 2 seconds and in particular within 1 second or less.

In some embodiments, volumetric swelling of the PD containing material upon reaction is less than about 400%, in particular less than about 100% and particularly less than about 50%. In some embodiments, the PD containing polymer swelling is a function of crosslinking density, polymer architecture, and PEG concentration. For instance, certain PD's may be more more reactive than others, meaning their crosslinking density would be increased.

Consequently, it would be expected that some of these PD's may swell less than others when similar polymer architectures and concentrations are used. In some embodiments, a further decrease in swelling may be achieved by adding more oxidant, which may result in greater crosslinking density. In some embodiments, the number of arms of the PEG will affect swelling as well as the molecular weight. For instance, a higher number of PEGylated arms for a given molecular weight, increases crosslinking density in the final hydrogel. Therefore, highly branched PEG derivatives may have lower swelling. PEG is a hydrophilic polymer that swells in aqueous media. Therefore, the more PEG in the final hydrogel, the higher the swelling may be. For instance, a 15 Wt % hydrogel will swell less than a 30 Wt % hydrogel. Furthermore, a 7.5 Wt % hydrogel will swell less than a 15 Wt % hydrogel. Accordingly, in some embodiments, swelling is a tunable property resulting from the PD, the oxidant concentration, the PEG architecture, and the PEG concentration.

The burst strength of the PD containing material upon reaction is between about 30 and about 300 mmHg, more particularly between about 60 and about 300 mmHg and particularly between about 100 and about 300 mmHg. It should be understood that the burst strength value may change depending on the testing apparatus used, the type of substrate used to test, the PD, the concentration of PD, the oxidant concentration, the Wt % polymer and the polymer architecture.

In some embodiments, blends of the compounds of the invention described herein, may be prepared with various polymers. Polymers suitable for blending with the compounds of the invention are selected to impart non-covalent interactions with the compound(s), such as hydrophobic-hydrophobic interactions or hydrogen bonding with an oxygen atom on PEG and a substrate surface. These interactions may increase the cohesive properties of the film to a substrate. In some embodiments, if a biopolymer is used it can introduce specific bioactivity to the film, (i.e., biocompatibility, cell binding, immunogenicity, etc.).

Suitable polymers include, for example, polyesters, PPG, linear PCL-diols (MW 600-2000), branched PCL-triols (MW 900), wherein PCL can be replaced with PLA, PGA, PLGA, and other polyesters, amphiphilic block (di, tri, or multiblock) copolymers of PEG and polyester or PPG, tri-block copolymers of PCL-PEG-PCL (PCL MW=500-3000, PEG MW=500-3000), tri-block copolymers of PLA-PEG-PLA (PCL MW=500-3000, PEG MW=500-3000), wherein PCL and PLA can be replaced with PGA, PLGA, and other polyesters. Pluronic polymers (triblock, diblock of various MW) and other PEG, PPG block copolymers are also suitable. Hydrophilic polymers with multiple functional groups (—OH, —NH2, —COOH) contained within the polymeric backbone such as PVA (MW 10,000-100,000), poly acrylates and poly methacrylates, polyvinylpyrrolidone, and polyethylene imines are also suitable. Biopolymers such as polysaccharides (e.g., dextran), hyaluronic acid, chitosan, gelatin, collagen, cellulose (e.g., carboxymethyl cellulose), alginate, proteins, PRP (platelet-rich plasma) etc. which contain functional groups can also be utilized.

Abbreviations: PCL=polycaprolactone, PLA=polylactic acid, PGA=Polyglycolic acid, PLGA=a random copolymer of lactic and glycolic acid, PPG=polypropyl glycol, and PVA=polyvinyl alcohol.

In some embodiments, blends of the invention include from about 0 to about 99.9% percent (by weight) of polymer to composition(s) of the invention, more particularly from about 1 to about 50 and even more particularly from about 1 to about 30.

In some embodiments, the compositions of the invention, either a blend or a compound of the invention per se, can be applied to suitable substrates using conventional techniques. Coating, dipping, spraying, spreading and solvent casting are possible approaches.

In some embodiments, the present invention provides antifouling coatings/constructs that are suitable for application in, for example, urinary applications. The coatings may be used anywhere that a reduction in bacterial attachment is desired: dental unit waterlines, implantable orthopedic devices, cardiovascular devices, wound dressings, percutaneous devices, surgical instruments, marine applications, food preparation surfaces and utensils.

In some embodiments, the present invention provides unique bioadhesive constructs that are suitable to repair or reinforce damaged tissue.

In some embodiments, suitable supports include those that can be formed from natural materials, such as collagen, metal surfaces such as titanium, iron, steel, etc. or man made materials such as polypropylene, polyethylene, polybutylene, polyesters, PTFE, PVC, polyurethanes and the like. In some embodiments, the support can be a solid surface such as a film, sheet, coupon or tube, a membrane, a mesh, a non-woven and the like. The support need only help provide a surface for the coating to adhere. In some embodiments, other suitable supports can be formed from a natural material, such as collagen, pericardium, dermal tissues, small intestinal submucosa and the like. The support can be a film, a membrane, a mesh, a non-woven and the like. The support need only help provide a surface for the bioadhesive/coating to adhere. The support should also help facilitate physiological reformation of the tissue at the damaged site. Thus the constructs of the invention provide a site for remodeling via fibroblast migration, followed by subsequent native collagen deposition. For biodegradable support of either biological or synthetic origins, degradation of the support and the adhesive can result in the replacement of the bioadhesive construct by the natural tissues of the patient.

In some embodiments, the coatings of the invention may include a compound of the invention or mixtures thereof or a blend of a polymer with one or more of the compounds of the invention. In one embodiment, the construct is a combination of a substrate, to which a blend is applied, followed by a layer(s) of one or more compounds of the invention. In another embodiment, two or more layers can be applied to a substrate wherein the layering can be combinations of one or more blends or one or more compositions of the invention. In some embodiments, the layering can alternate between a blend and a composition layer or can be a series of blends followed by a composition layer or vice versa. In some embodiments, the loading density of the coating layer is from about 0.001 g/m² to about 200 g/m², more particularly from about 5 g/m² to about 150 g/m², and more particularly from about 10 g/m² to about 100 g/m². Thus, In some embodiments, a coating has a thickness of from about 1 to about 200 nm. In other embodiments, the thickness of the film is from about 1 to about 200 microns.

### EXPERIMENTAL EXAMPLES

### Example 1

### Synthesis of Acetyl Vanillic Acid

20.04 g (112 mmol) of vanillic acid was dissolved in 50 mL (618 mmol) of pyridine and 50 mL (529 mmol) of acetic anhydride and allowed to stir for 2 hour. The solution was poured into 1200 mL of nanopure water and the pH was adjusted to 2 using concentrated HCl. The solution was extracted twice with a total of 700 mL of ethyl acetate and dried with anhydrous magnesium sulfate. The magnesium sulfate was suction filtered off and the organic solvent was evaporated off. The compound was dried for ˜23 hours under vacuum. The compound was recrystallized in 400 mL of a 1:1 mixture of water:methanol. The precipitate was suction filtered and placed under vacuum. 21.58 g of material was obtained. ¹H NMR (400 MHz, DMSO/TMS): δ 13.08 (s, 1H, —COOH—), 7.59 (d, 1H, —C₆H₃—), 7.55 (s, 1H, —C₆H₃—), 7.20 (d, 1H, —C₆H₃—), 6.55 (d, 1H, —CH═CH—COOH), 3.81 (s, 3H, —CH₃—O—C₆H₃—), 2.27 (s, 3H, CH₃—COO—C₆H₃—).

### Example 2

### Synthesis of Acetyl Ferulic Acid

20.0 g (103 mmol) of ferulic acid was dissolved in 50 mL (618 mmol) of pyridine and 50 mL (529 mmol) of acetic anhydride and allowed to stir for 90 minutes. The solution was poured into 1200 mL of nanopure water and the pH was adjusted to 2 using concentrated HCl. The solution was extracted twice with a total of 800 mL of ethyl acetate. The insoluble material from the aqueous layer was suction filtered. The insoluble material was dried for ˜20 hours and sonicated in 400 mL nanopure water for 45 minutes. The material was suction filtered, washed with 100 mL nanopure water and dried under vacuum for ˜23 hours. 14.1 g of material was heated and stirred in 500 mL of methanol and placed at ˜15° C. for ˜22 hours. The methanol was decanted off and 200 mL of methanol was added and stirred for ˜15 minutes. The precipitate was suction filtered and placed under vacuum until dry. 11.49 g of material was obtained. ¹H NMR (400 MHz, DMSO/TMS): δ 12.37 (s, 1H, —COOH—), 7.54 (d, 1H, —CH═CH—COOH), 7.44 (s, 1H, —C₆H₃—), 7.23 (d, 1H, —C₆H₃—), 7.07 (d, 1H, —C₆H₃—), 6.55 (d, 1H, —CH═CH—COOH), 3.79 (s, 3H, —CH₃—O—C₆H₃—), 2.23 (s, 3H, CH₃—COO—C₆H₃—).

### Example 3

### Synthesis of Boc-4-amino-3-Acetoxybenzoic acid

300 mL of 0.4M NaHCO₃ was added to 10.1 g (65.3 mmol) of 4-amino-3-hydroxybenzoic acid. The reaction was purged with argon for 20 minutes. 14.97 g (68.6 mmol) of Boc-Anhydride was dissolved in 150 mL of THF. The THF/Boc-Anhydride solution was added to the aqueous solution and bubbled with argon while stirring for 20 hours. The solution was suction filtered and the THF was roto evaporated off. The aqueous mixture was acidified to a pH of 2 with concentrated HC (11 mL). The mixture was washed 3 times with a total of 1200 mL of ethyl acetate. The ethyl acetate was roto evaporated off and the compound was then dried for 2 hours under vacuum. The compound was then heated at 72° C. with agitation in 150 mL of ethyl acetate. The solution was placed at −15° C. for 1 hour and the precipitate was washed with 100 mL of ethyl acetate. The insoluble material was suction filtered off and placed under vacuum until dry (called LN011055A). The material in the organic extract was isolated by roto evaporating off the ethyl acetate and placing under vacuum until dry (called LN011055B). 3.05 g of LN011055A was heated with stirring in 150 mL of nanopure water and 100 mL of methanol. The mixture was placed at 4° C. for 3 hours and the precipitate was suction filtered off and dried (2.173 g obtained). LN011055B was heated with stirring in 300 mL of nanopure water and 200 mL of methanol. This was placed at 4° C. for 3 hours. The precipitate was suction filtered and dried (3.749 g obtained). 1H NMR showed LN011055A and B to be the same and they were combined. 5.94 g of Boc-4-amino-3-hydroxybenzoic acid was obtained (LN011055). 1.42 g (23.5 mmol) of Boc-4-amino-3-hydroxybenzoic acid was dissolved in 15 mL (185 mmol) of pyridine and 2.75 mL (159 mmol) of acetic anhydride. The reaction was stirred for ˜2 hour. The reaction was poured into 400 mL nanopure water and the pH was adjusted to 2 with 15 mL of concentrated HCl. This was extracted three times with a total of 600 mL ethyl acetate. The organic extract was roto-evaporated off. This was placed under vacuum for 20 hours. To this was added 400 mL of nanopure water. The mixture was heated with stirring and placed at 4° C. for ˜3 hours. The precipitate was suction filtered and placed under vacuum for ˜19 hours. The compound was heated in 250 mL of nanopure water with stirring and placed at 4° C. for 6 hours.

The precipitate was suction filtered and washed with 250 mL of cold nanopure water. The compound was placed under vacuum for ˜22 hours. The compound was then frozen and freeze dried to remove moisture. 4.7 g of material was obtained (LN011066). ¹H NMR (400 MHz, DMSO/TMS): δ 12.89 (s, 1H, —C₆H₃—COOH—), 9.26 (s, 1H, —C₆H₃—NH-Boc), 7.98 (d, 1H, —C₆H₃—), 7.74 (d, 1H, —C₆H₃—), 7.61 (s, 1H, —C₆H₃—), 2.30 (s, 3H, —COCH₃), 1.50 (s, 9H, —NH—COOC(CH₃)₃).

### Example 4

### Synthesis of 4-Acetoxy-3-nitrophenylacetic Acid

9.7 g (49 mmol) of 4-hydroxy-3-nitrophenylacetic acid was dissolved in 25 mL (309 mmol) of pyridine and 25 mL (265 mmol) of acetic anhydride and allowed to stir for 2 hours. The solution was poured into 600 mL of nanopure water and the pH was adjusted to 2 using concentrated HCl (27 mL). The solution was extracted three times with a total of 600 mL of ethyl acetate. The solvent was roto-evaporated off and the compound was dried under vacuum for 19 hours. The compound was heated with stirring in 250 mL of a 1:1 mixture of nanopure water:methanol. The solution was placed at −15° C. for ˜2 hours. The precipitate was suction filtered and washed with ˜250 mL of cold nanopure water (LN011074A-impure). The filtrate was placed at −15° C. for ˜19 hours and then placed at 4° C. for ˜5 hours. The precipitate was suction filtered and placed under vacuum until dry (LN011074B). LN011074B was added to 150 mL nanopure water and heated with stirring. 25 mL of methanol was added when solution began to steam. The solution was placed at 4° C. for ˜3 hours. The solution was filtered and placed at −20° C. for ˜2 hours and then placed at 4° C. for ˜16 hours. The precipitate was suction filtered, washed with 100 mL of nanopure water and dried under vacuum until dry. This material was then heated with stirring in nanopure water until it began to steam. 25 mL of methanol was added to the solution. The solution was gravity filtered and placed at 4° C. for ˜22 hours. The precipitate was suction filtered and placed under vacuum for ˜24 hours. 1.31 g of material was obtained (LN011074). ¹H NMR (400 MHz, DMSO/TMS): δ 12.6 (s, 1H, —CH₂—COOH—), 8.08 (s, 1H, —C₆H₃—), 7.71 (d, 1H, —C₆H₃—), 7.41 (d, 1H, —C₆H₃—), 3.78 (s, 2H, —CH₂—COOH), 2.33 (s, 3H, —COCH₃).

### Example 5

### Synthesis of Boc-3-amino-4-Acetoxybenzoic acid

430 mL of 0.4M NaHCO₃ was added to 14.91 g (97.9 mmol) of 3-amino-4-hydroxybenzoic acid. The reaction was purged with argon for 30 minutes. 22.92 g (103 mmol) of Boc-Anhydride was dissolved in 150 mL of THF. The THF/Boc-Anhydride solution was added to the aqueous solution and bubbled with argon while stirring for 24 hours. The THF was roto evaporated off. The aqueous mixture was acidified to a pH of 2 with concentrated HCl (17 mL). The mixture was washed 3 times with a total of 600 mL of ethyl acetate. The ethyl acetate was roto evaporated off and the compound was then dried for 4 hours under vacuum. The compound was then heated in 250 mL of nanopure water until steam was observed. 410 mL of methanol was added to the solution. The solution was filtered and placed at 4° C. for ˜22 hours. 200 mL of nanopure water and 150 mL of methanol was added to the solution. The solution was placed at −15° C. for 4 days. No precipitate was observed so the methanol was roto evaporated off. The aqueous solution was placed at −15° C. for ˜16 hours. The insoluble material was suction filtered off and placed under vacuum until dry. ¹H NMR showed the compound to be pure. 13.87 g of Boc-3-amino-4-hydroxybenzoic acid was obtained (LN011401). 13.87 g (55 mmol) of Boc-3-amino-4-hydroxybenzoic acid was dissolved in 35 mL (433 mmol) of pyridine and 35 mL (370 mmol) of acetic anhydride. The reaction was stirred for 1 hour. The reaction was poured into 500 mL nanopure water and the pH was adjusted to 2 with 35 mL of concentrated HCl. This was extracted two times with a total of 300 mL ethyl acetate. The organic extract was roto evaporated off. This was placed under vacuum for 90 minutes. To this was added 250 mL of nanopure water. The mixture was heated with stirring until steam was noticed. 325 mL of methanol was added to the solution. The solution was gravity filtered and placed at 4° C. for ˜3 days. The precipitate was suction filtered and washed with 100 mL nanopure water. The precipitate was placed under vacuum for ˜20 hours. 11.27 g of pure compound was obtained (LN011426). H NMR (400 MHz, DMSO/TMS): δ 12.8 (s, 1H, —C₆H₃—COOH—), 9.10 (s, 1H, —C₆H₃—NH-Boc), 8.38 (s, 1H, —C₆H₃—), 7.61 (d, 1H, —C₆H₃—), 7.18 (d, 1H, —C₆H₃—), 2.28 (s, 3H, —COCH₃), 1.47 (s, 9H, —NH—COOC(CH₃)₃).

### Example 6

### Synthesis of 3,4,5-Triacetoxybenzoic Acid

20.01 g (118 mmol) of gallic acid was dissolved in 100 mL (1.236 mmol) of pyridine and 100 mL (1.058 mmol) of acetic anhydride and allowed to stir for 2 hours. The solution was poured into 1500 mL of nanopure water and the pH was adjusted to 2 using concentrated HC (110 mL). The solution was extracted three times with a total of 600 mL of ethyl acetate. The ethyl acetate was roto-evaporated off and the compound was placed under vacuum for ˜3 days. 250 mL of nanopure water was added to the compound and heat was applied until the resulting solution began to steam. 50 mL of methanol was slowly added to the solution. The solution was gravity filtered and placed at 4° C. for ˜2 days. The precipitate was suction filtered and placed under vacuum for ˜2 days. 250 mL of nanopure water was added to the compound and heat was applied until the resulting solution began to steam. 75 mL of methanol was slowly added to the solution. The solution was gravity filtered and placed at 4° C. for ˜3 days. The precipitate was suction filtered, washed with 100 mL nanopure water, and placed under vacuum until dry. The resulting compound was dissolved in 75 mL of methanol with heat and stirring. To this was added 75 mL of nanopure water. This was placed at −15° C. for ˜28 hours. The precipitate was suction filtered, washed with 150 mL nanopure water and dried under vacuum. The compound was dissolved again in 75 mL of methanol. Once dissolved, 75 mL of methanol was added. The solution was placed at 4° C. for 3 days. The precipitate was suction filtered and placed under vacuum until dry. 5.738 g of material was obtained (LN011438). ¹H NMR (400 MHz, DMSO/TMS): δ 13.44 (s, 1H, —COOH—), 7.75 (s, 2H, —C₆H₃—), 2.27 (t, 9H, CH₃—COO—C₆H₃—).

### Example 7

### Synthesis of 3,4-Diacetoxycaffeic Acid

14.979 g (83.1 mmol) of caffeic acid was dissolved in 75 mL (927 mmol) of pyridine and 75 mL (794 mmol) of acetic anhydride and allowed to stir for 75 minutes. The solution was poured into 500 mL of nanopure water and the pH was adjusted to 2 using concentrated HCl (77.5 mL). The solution was extracted two times with a total of 450 mL of ethyl acetate. The ethyl acetate was roto evaporated off and the compound was placed under vacuum for ˜3 hours. 250 mL of nanopure water was added to the compound and heat was applied until the resulting solution began to steam. 500 mL of methanol was slowly added to the solution. The solution was gravity filtered and placed at 4° C. for ˜3 days. The precipitate was suction filtered, washed with 100 mL nanopure water and placed under vacuum for 28 hours. 17.08 g of material was obtained (LN011424). ¹H NMR (400 MHz, DMSO/TMS): δ 12.47 (s, 1H, —COOH—), 7.66-7.54 (m, 3H, —C₆H₃—CH═CH—COOH), 7.30 (d, 1H, —C₆H₃—), 6.52 (d, 1H, —CH═CH—COOH), 2.28 (d, 6H, CH₃—COO—C₆H₃—).

### Example 8

### Synthesis of Di-Boc-3,4-diaminobenzoic acid

1150 mL of 0.4M NaHCO₃ was added to 38.02 g (250 mmol) of 3,4-diaminobenzoic acid. The reaction was purged with argon. 117.4 g (˜530 mmol) of Boc-Anhydride was dissolved in 575 mL of THF. The THF/Boc-Anhydride solution was added to the aqueous solution and stirred under argon for 20 hours. The solution was filtered and the THF was roto evaporated off. The aqueous mixture was acidified to a pH of 2 with concentrated HCl (40 mL). The precipitate was suction filtered off and washed with nanopure water. The compound was transferred to an appropriately sized flask and heated in 1 L of nanopure water. 850 mL of methanol was slowly added until all material was dissolved. The solution was placed at 4° C. for 21 hours. The precipitate was suction filtered off and dried under vacuum for 23 hours. The compound was removed from vacuum and dissolved in 500 mL of methanol with heat and stirring. The solution was placed at −15° C. for 20 minutes. 500 mL of nanopure water was added to the solution and the solution was placed at 4° C. for 2 hours. The precipitate was suction filtered off and washed with 300 mL of nanopure water. The compound was placed under vacuum for 18 hours. 39.77 g of Di-Boc-3,4-diaminobenzoic acid was obtained (LN012131). ¹H NMR (400 MHz, DMSO/TMS): δ 8.71 (d, 1H, —C₆H₃—), 8.66 (d, 1H, —C₆H₃—), 8.04 (s, 1H, —C₆H₃—), 7.66 (d, 1H, —C₆H₃—NH-Boc), 7.60 (d, 1H, —C₆H₃—NH-Boc), 1.44 (s, 18H, —NHCOOC(CH₃)₃).

### Example 9

### Synthesis of Diacetyl-dopamine (Ac₂-dopamine)

24 g (126.3 mmol) of dopamine HCl was placed in a 500 mL round bottom flask. 150 mL of 33% HBr solution was added along with 125 mL of acetic chloride. The reaction was allowed to stir overnight at room temperature. The reaction was bubbled with argon for 2 hours to remove excess acid (equipped with a trap containing potassium hydroxide). The reaction was added to 1.4 L of diethyl ether and placed at 4° C. overnight. The solvent was decanted off and the resulting compound was placed under vacuum until dry. The compound was dissolved in 100 mL of ethanol and added to 800 mL of diethyl ether and placed at 4° C. overnight. The solvent was decanted and the resulting compound was dried under vacuum overnight. ¹H NMR confirmed the chemical structure. 33.9 g of Diacetyl-dopamine was obtained (LN002301).

### Example 10

### Synthesis of Surphys-054 (MPEG5k-(HFA))

5.01 g (0.5 mmol) of MPEG5k-(NH₂), 0.324 g (1.6 mmol) of hydroferulic acid and 0.627 g (1.6 mmol) of HBTU was dissolved in 50 mL DMF and 25 mL of chloroform while stirring. 0.362 mL (2.6 mmol) of triethylamine was added and the reaction was allowed to stir for ˜90 minutes. The reaction was gravity filtered into 350 mL of diethyl ether and placed at ˜4° C. for ˜23 hours. The precipitate was suction filtered and dried under vacuum for 19 hours. 4.9 g of MPEG5k-(HFA) was dissolved in 49 mL of nanopure water. This solution was suction filtered, poured into 2000MWCO dialysis tubing, and placed in nanopure water (1 L) acidified with concentrated HCl (0.1 mL). The dialysate was changed 8 times over the next 49 hours. The dialysate was changed to nanopure water (1 L) and changed 4 times over the next 3 hours. The solution was suction filtered, frozen and placed on a lyophilizer. 2.239 g of material was obtained. ¹H NMR (400 MHz, D2O/TMS): δ 6.77 (s, 1H, —C₆H₃—), 6.70 (d, 1H, —C₆H₃—), 6.60 (d, 1H, —C₆H₃—), 3.8-3.0 (m, 458H, PEG, —C₆H₃—O—CH₃), 2.73 (t, 2H, —NHCOCH₂CH₂—), 2.39 (t, 2H, —NHCOCH₂CH₂—).

### Example 11

### Synthesis of Surphys-059 (PEG20k-(PABA)₈)

15.00 g (0.75 mmol) of PEG20k-(NH₂)₈, 1.713 g (7.2 mmol) of 4-Boc-aminobenzoic acid and 2.739 g (7.2 mmol) of HBTU was dissolved in 150 mL DMF and 75 mL of chloroform while stirring. 1.84 mL (13.2 mmol) of triethylamine was added and the reaction was allowed to stir for ˜4 hours. The reaction was gravity filtered into 1.2 L of diethyl ether and placed at ˜4° C. for ˜24 hours. The precipitate was suction filtered and dried under vacuum for 2 days. The intermediate was called PEG20k-(Boc-4-ABA)₈. 15.5 g of PEG20k-(Boc-4-ABA)₈ was dissolved in 3 mL of chloroform. 3 mL of trifluoroacetic acid was slowly added to the solution and allowed to stir for 30 minutes. The solution was roto evaporated at ˜30-35° C. until ˜30-50% of the volume was removed. The solution was then poured into 1.2 L of diethyl ether and placed at 4° C. for ˜19 hours. The precipitate was suction filtered and transferred to a beaker.

This was placed under vacuum for ˜3 days. 11.7 g of polymer was obtained and dissolved in 117 mL of nanopure water. This solution was suction filtered, poured into 2000MWCO dialysis tubing, and placed in 3 L of nanopure water. The dialysate was changed twice over a period of 3 hours. The dialysate was changed to nanopure water (3 L) acidified with concentrated HCl (0.3 mL). The dialysate was changed 8 times over the next 43 hours. The dialysate was changed to nanopure water (3 L) and changed 4 times over the next 3 hours. The solution was suction filtered, frozen and placed on a lyophilizer. 8.17 g of material was obtained (LN010271). The synthesis did not fully deprotect the Boc protecting group. 8.04 g of material was dissolved in 16 mL of chloroform and 16 mL of trifluoroacetic acid was slowly added. The reaction was stirred for 30 minutes The reaction was poured into 400 mL of diethyl ether and placed at 4° C. for 18 hours. The precipitate was suction filtered and placed under vacuum for ˜23 hours. 7.75 g of polymer was obtained and dissolved in 150 mL of nanopure water. This solution was suction filtered, poured into 2000MWCO dialysis tubing, and placed in 1 L of nanopure water. The dialysate was changed twice over a period of 4 hours. The dialysate was changed to nanopure water (1 L) acidified with concentrated HCl (0.1 mL). The dialysate was changed 8 times over the next 43 hours. The dialysate was changed to nanopure water (1 L) and changed 4 times over the next 3 hours. The solution was suction filtered, frozen and placed on a lyophilizer. 5.37 g of material was obtained (LN010559). ¹H NMR (400 MHz, D2O/TMS): δ 7.52 (d, 2H, —C₆H₃—), 6.73 (d, 2H, —C₆H₃—), 3.8-3.2 (m, 226H, PEG).

### Example 12

### Synthesis of Surphys-060 (MPEG5k-(PABA))

5.085 g (1 mmol) of MPEG5k-NH₂, 0.577 g (2.4 mmol) of 4-Boc-aminobenzoic acid and 0.912 g (2.4 mmol) of HBTU was dissolved in 50 mL of DMF and 25 mL of chloroform while stirring. 0.446 mL (4.4 mmol) of triethylamine was added and the reaction was allowed to stir for ˜90 minutes. The reaction was gravity filtered into 400 mL of diethyl ether and placed at ˜4° C. for ˜22 hours. The precipitate was suction filtered and dried under vacuum for 2 days (LN010538). The intermediate was called MPEG5k-(Boc-4-ABA). 5.01 g of MPEG5k-(Boc-4-ABA) was dissolved in 10 mL of chloroform. 10 mL of trifluoroacetic acid was slowly added to the solution and allowed to stir for 30 minutes. The solution was roto evaporated at ˜30-35° C. until ˜30-50% of the volume was removed. The solution was then poured into 200 mL of diethyl ether and placed at 4° C. for ˜22 hours. The precipitate was suction filtered and transferred to a beaker. This was placed under vacuum for ˜23 hours. 4.2 g of polymer was obtained and dissolved in 42 mL of nanopure water. This solution was suction filtered, poured into 2000MWCO dialysis tubing and placed in 1 L of nanopure water. The dialysate was changed twice over a period of 3 hours. The dialysate was changed to nanopure water (1 L) acidified with concentrated HCl (0.1 mL). The dialysate was changed 8 times over the next 42 hours. The dialysate was changed to nanopure water (1 L) and changed 4 times over the next 3 hours. The solution was suction filtered, frozen and placed on a lyophilizer. 1.88 g of material was obtained. ¹H NMR (400 MHz, D2O/TMS): δ 7.52 (d, 2H, —C₆H₃—), 6.73 (d, 2H, —C₆H₃—), 3.8-3.2 (m, 455H, PEG).

### Example 13

### Synthesis of Surphys-061 (PEG20k-(HFA)₈)

10.00 g (0.5 mmol) of PEG20k-(NH₂)₈, 0.8322 g (4.2 mmol) of hydroferulic acid and 1.595 g (4.2 mmol) of HBTU was dissolved in 100 mL DMF and 50 mL of chloroform while stirring. 1.14 mL (8.2 mmol) of triethylamine was added and the reaction was allowed to stir for ˜90 minutes. The reaction was gravity filtered into 700 mL of diethyl ether and placed at ˜4° C. for ˜20 hours. The precipitate was suction filtered and dried under vacuum for 5 hours. 10.5 g of PEG20k-(HFA)₈ was dissolved in 100 mL of nanopure water. This solution was suction filtered, poured into 2000MWCO dialysis tubing, and placed in nanopure water (3 L) acidified with concentrated HCl (0.2 mL). The dialysate was changed 8 times over the next 42 hours. The dialysate was changed to nanopure water (2 L) and changed 4 times over the next 3 hours. The solution was suction filtered, frozen and placed on a lyophilizer. 7.78 g of material was obtained. ¹H NMR (400 MHz, D2O/TMS): δ 6.77 (s, 1H, —C₆H₃—), 6.70 (d, 1H, —C₆H₃—), 6.60 (d, 1H, —C₆H₃—), 3.8-3.0 (m, 229H, PEG, —C₆H₃—O—CH₃), 2.73 (t, 2H, —NHCOCH₂CH₂—), 2.39 (t, 2H, —NHCOCH₂CH₂—).

### Example 14

### Synthesis of Surphys-062 (PEG20k-(3-Methoxy-PABA)₈)

14.99 g (0.75 mmol) of PEG20k-(NH₂)₈, 2.575 g (9.6 mmol) of 4-Boc-amino-3-methoxybenzoic acid and 3.657 g (9.6 mmol) of HBTU was dissolved in 150 mL of DMF and 75 mL of chloroform while stirring. 2.175 mL (15.6 mmol) of triethylamine was added and the reaction was allowed to stir for ˜90 minutes. The reaction was gravity filtered into 1.2 L of diethyl ether and placed at ˜4° C. for ˜23 hours. The precipitate was suction filtered and dried under vacuum for 4 days (LN010526). The intermediate was called PEG20k-(Boc-4A-3MBA)₈. 16.45 g of PEG20k-(Boc-4A-3-MBA)₈ was dissolved in 33 mL of chloroform. 33 mL of trifluoroacetic acid was slowly added to the solution and allowed to stir for 30 minutes. The solution was roto-evaporated at ˜30-35° C. until ˜30-50% of the volume was removed. The solution was then poured into 400 mL of diethyl ether and placed at 4° C. for 90 minutes. The precipitate was suction filtered and transferred to a beaker. This was placed under vacuum for ˜15 hours. 18.26 g of polymer was obtained and dissolved in 180 mL of nanopure water. This solution was suction filtered, poured into 2000 MWCO dialysis tubing, and placed in 3 L of nanopure water. The dialysate was changed twice over a period of 3 hours. The dialysate was changed to nanopure water (3 L) acidified with concentrated HCl (0.3 mL). The dialysate was changed 8 times over the next 44 hours. The dialysate was changed to nanopure water (3 L) and changed 4 times over the next 3 hours. The solution was suction filtered, frozen and placed on a lyophilizer. 9.23 g of material was obtained. ¹H NMR (400 MHz, D2O/TMS): δ 7.21 (s, 1H, —C₆H₃—), 7.18 (d, 1H, —C₆H₃—), 6.75 (d, 1H, —C₆H₃—), 3.8-3.2 (m, 229H, PEG, —C₆H₃—OCH₃).

### Example 15

### Synthesis of Surphys-064 (MPEG5k-(4A-3MBA))

4.012 g (0.8 mmol) of MPEG5k-(NH₂), 0.26 g (1 mmol) of 4-Boc-amino-3-methoxybenzoic acid and 0.383 g (1 mmol) of HBTU was dissolved in 40 mL of DMF and 20 mL of chloroform while stirring. 0.269 mL (1.93 mmol) of triethylamine was added and the reaction was allowed to stir for ˜3 hours. The reaction was gravity filtered into 350 mL of diethyl ether and placed at ˜4° C. for ˜7 hours. The precipitate was suction filtered and dried under vacuum for 13 hours (LN010578). The intermediate was called MPEG5k-(Boc-4A-3MBA). 4.0 g of MPEG5k-(Boc-4A-3-MBA) was dissolved in 8 mL of chloroform. 8 mL of trifluoroacetic acid was slowly added to the solution and allowed to stir for 30 minutes. The solution was then poured into 350 mL of diethyl ether and placed at 4° C. for 19 hours. The precipitate was suction filtered and transferred to a beaker. This was placed under vacuum for ˜25 hours. The polymer was dissolved in 100 mL of nanopure water. This solution was suction filtered, poured into 2000MWCO dialysis tubing, and placed in 1.5 L of nanopure water. The dialysate was changed twice over a period of 3 hours. The dialysate was changed to nanopure water (1.5 L) acidified with concentrated HCl (0.15 mL). The dialysate was changed 8 times over the next 23 hours. The dialysate was changed to nanopure water (1.5 L) and changed 4 times over the next 3 hours. The solution was suction filtered, frozen and placed on a lyophilizer. 2.78 g of material was obtained. ¹H NMR (400 MHz, D2O/TMS): δ 7.21 (s, 1H, —C₆H₃—), 7.18 (d, 1H, —C₆H₃—), 6.75 (d, 1H, —C₆H₃—), 3.8-3.2 (m, 458H, PEG, —C₆H₃—OCH₃).

### Example 16

### Synthesis of Surphys-065 (PEG20k-(3,4-DABA)₈)

14.94 g (0.75 mmol) of PEG20k-(NH₂)₈, 3.394 g (9.6 mmol) of Di-Boc-3,4-diaminobenzoic acid and 3.659 g (9.6 mmol) of HBTU was dissolved in 150 mL of DMF and 75 mL of chloroform while stirring. 2.175 mL (15.6 mmol) of triethylamine was added and the reaction was allowed to stir for ˜3 hours. The reaction was gravity filtered into 1.2 L of diethyl ether and placed at ˜4° C. for ˜20 hours. The precipitate was suction filtered and dried under vacuum for 24 hours (LN010580). The intermediate was called PEG20k-(Di-Boc-3,4-DABA)₈. 17.62 g of PEG20k-(Di-Boc-3,4-DABA)₈ was dissolved in 71 mL of chloroform. 71 mL of trifluoroacetic acid was slowly added to the solution and allowed to stir for 55 minutes. The solution was then poured into 3 L of a 1:1 diethyl ether:heptane mix and placed at 4° C. for 4 hours. The precipitate was suction filtered and transferred to a beaker. This was placed under vacuum for ˜21 hours, then dissolved in 300 mL of nanopure water. This solution was suction filtered, poured into 2000MWCO dialysis tubing, and placed in 3 L of nanopure water. The dialysate was changed twice over a period of 3 hours. The dialysate was changed to nanopure water (3 L) acidified with concentrated HCl (0.3 mL). The dialysate was changed 8 times over the next 24 hours. The dialysate was changed to nanopure water (3 L) and changed 4 times over the next 3 hours. The solution was suction filtered, frozen and placed on a lyophilizer. 8.00 g of material was obtained. ¹H NMR (400 MHz, D2O/TMS): δ 7.17 (s, 1H, —C₆H₃—), 7.14 (d, 1H, —C₆H₃—), 6.74 (d, 1H, —C₆H₃—), 3.8-3.2 (m, 226H, PEG).

### Example 17

### Synthesis of Surphys-066 (MPEG5k-(3,4-DABA))

4.034 g (0.8 mmol) of MPEG5k-(NH₂), 0.3534 g (1 mmol) of Di-Boc-3,4-Diaminobenzoic acid and 0.3877 g (1 mmol) of HBTU was dissolved in 40 mL of DMF and 20 mL of chloroform while stirring. 0.274 mL (1.97 mmol) of triethylamine was added and the reaction was allowed to stir for ˜3 hours. The reaction was gravity filtered into 350 mL of diethyl ether and placed at ˜4° C. for ˜6 hours. The precipitate was suction filtered and dried under vacuum for 25 hours (LN010582). The intermediate was called MPEG5k-(Di-Boc-3,4-DABA). 4.17 g of MPEG5k-(Di-Boc-3,4-DABA) was dissolved in 17 mL of chloroform. 17 mL of trifluoroacetic acid was slowly added to the solution and allowed to stir for 55 minutes. The solution was then poured into 350 mL of diethyl ether and 100 mL of heptane and placed at 4° C. for 21 hours. The precipitate was suction filtered and transferred to a beaker. This was placed under vacuum for ˜24 hours. The polymer was dissolved in 100 mL of nanopure water. This solution was suction filtered, poured into 2000MWCO dialysis tubing, and placed in 1.5 L of nanopure water. The dialysate was changed twice over a period of 3 hours. The dialysate was changed to nanopure water (1.5 L) acidified with concentrated HCl (0.15 mL). The dialysate was changed 8 times over the next 23 hours. The dialysate was changed to nanopure water (3.5 L) and changed 4 times over the next 3 hours. The solution was suction filtered, frozen and placed on a lyophilizer. 1H NMR (400 MHz, D2O/TMS): δ 7.17 (s, 1H, —C₆H₃—), 7.14 (d, 1H, —C₆H₃—), 6.74 (d, 1H, —C₆H₃—), 3.8-3.2 (m, 455H, PEG).

### Example 18

### Synthesis of Surphys-068 (PEG20k-(FA)₈)

14.98 g (0.75 mmol) of PEG20k-(NH₂)₈, 2.29 g (9.6 mmol) of acetyl ferulic acid and 3.657 g (9.6 mmol) of HBTU was dissolved in 150 mL of DMF and 75 mL of chloroform while stirring. 2.174 mL (15.6 mmol) of triethylamine was added and the reaction was allowed to stir for ˜3 hours. The reaction was gravity filtered into 800 mL of a 1:1 diethyl ether:heptane mix and placed at ˜15° C. for ˜16 hours. The precipitate was suction filtered and dried under vacuum for 27 hours (LN011045). The intermediate was called PEG20k-(AFA)₈. Coupling efficiency was ˜75-80% according to ¹H NMR (based on Aromatic:PEG peak ratio). ˜15 g of this material was dissolved in 150 mL DMF and 75 mL of chloroform with 0.943 g of HBTU and 0.58 g of acetyl ferulic acid. 0.34 mL of triethylamine was added and the reaction was allowed to proceed for ˜3 hours. The reaction was gravity filtered into 800 mL of a 1:1 diethyl ether:heptane mix and placed at −15° C. for ˜2 days. The precipitate was suction filtered and placed under vacuum for ˜22 hours. This material was dissolved in 120 mL of anhydrous DMF. Argon was bubbled through the reaction for 30 minutes. 8 mL of piperidine was added to the reaction with argon bubbling through. The reaction was stirred for 30 minutes. The reaction was gravity filtered into a 1:1 MTBE:Heptane mix and placed at −15° C. for 20 hours. The precipitate was dried under vacuum for ˜2 hours. The polymer was dissolved in 300 mL of nanopure water with 0.230 mL of concentrated HCl. The polymer solution was poured into 2000 MWCO dialysis tubing and dialyzed against 3 L of nanopure water containing 0.3 mL of concentrated HCl. The dialysate was changed 6 times over the next 24 hours. The dialysate was changed to nanopure water (3 L) and changed 4 times over the next 7 hours. The polymer solution was suction filtered, frozen and placed on a lyophilizer. 12.2 g of material was obtained (LN011051). Piperidine was still present, so the polymer was dissolved in 250 mL of nanopure water and poured into 2000 MWCO dialysis tubing. The solution was dialyzed against 3 L of nanopure water containing (0.3 mL) of concentrated HCl. The dialysate was changed 3 times over 16 hours. The dialysate was changed to nanopure water. The dialysate was changed 4 times over the next ˜4 hours. The solution was frozen and placed on a lyophilizer. 11.56 g of material was obtained (LN011068). ¹H NMR (400 MHz, D2O/TMS): δ 7.28 (d, 1H, —C₆H₃—CH═CH—), 7.1 (s, 1H, —C₆H₃—), 7.00 (d, 1H, —C₆H₃—), 6.76 (d, 1H, —C₆H₃—), 6.36 (d, 1H, —C₆H₃—CH═CH—), 3.8-3.2 (m, 229H, PEG, —C₆H₃—OCH₃).

### Example 19

### Synthesis of Surphys-069 (PEG20k-(VA)₈)

14.99 g (0.75 mmol) of PEG20k-(NH₂)₈, 2.044 g (9.6 mmol) of acetyl vanillic acid and 3.682 g (9.6 mmol) of HBTU was dissolved in 150 mL of DMF and 75 mL of chloroform while stirring. 2.174 mL (15.6 mmol) of triethylamine was added and the reaction was allowed to stir for ˜3 hours. The reaction was gravity filtered into 800 mL of a 1:1 diethyl ether:heptane mix and placed at −15° C. for ˜16 hours. The precipitate was suction filtered and dried under vacuum for 27 hours (LN011047). The intermediate was called PEG20k-(AVA)₈. Coupling efficiency was ˜75-80% according to ¹H NMR (based on Aromatic:PEG peak ratio). ˜15 g of this material was dissolved in 150 mL DMF and 75 mL of chloroform with 0.956 g of HBTU and 0.519 g of acetyl vanillic acid. 0.34 mL of triethylamine was added and the reaction was allowed to proceed for ˜3 hours. The reaction was gravity filtered into 800 mL of a 1:1 diethyl ether:heptane mix and placed at −15° C. for ˜2 days. The precipitate was suction filtered and placed under vacuum for ˜22 hours. This material was dissolved in 120 mL of anhydrous DMF. Argon was bubbled through the reaction for 30 minutes. 8 mL of piperidine was added to the reaction with argon bubbling through. The reaction was stirred for 30 minutes. The reaction was gravity filtered into a 1:1 MTBE:Heptane mix and placed at −15° C. for 20 hours. The precipitate was dried under vacuum for ˜2 hours. The polymer was dissolved in 300 mL of nanopure water with 0.700 mL of concentrated HCl. The polymer solution was poured into 2000 MWCO dialysis tubing and dialyzed against 3 L of nanopure water containing 0.3 mL of concentrated HCl. The dialysate was changed 6 times over the next 24 hours. The dialysate was changed to nanopure water (3 L) and changed 4 times over the next 7 hours. The polymer solution was suction filtered, frozen and placed on a lyophilizer. 12.15 g of material was obtained (LN011053). Piperidine was still present, so polymer was dissolved in 250 mL of nanopure water and poured into 2000 MWCO dialysis tubing. The solution was dialyzed against 3 L of nanopure water containing (0.3 mL) of concentrated HCl. The dialysate was changed 3 times over 16 hours. The dialysate was changed to nanopure water. The dialysate was changed 4 times over the next ˜4 hours. The solution was frozen and placed on a lyophilizer. 11.72 g of material was obtained (LN011069). ¹H NMR (400 MHz, D2O/TMS): δ 7.26 (s, 1H, —C₆H₃—), 7.19 (d, 1H, —C₆H₃—), 6.81 (d, 1H, —C₆H₃—), 3.8-3.2 (m, 229H, PEG, —C₆H₃—OCH₃).

### Example 20

### Synthesis of Surphys-070 (MPEG5k-(FA))

4.98 g (1 mmol) of MPEG5k-(NH₂), 0.396 g (1.6 mmol) of Acetyl Ferulic Acid and 0.614 g (1.6 mmol) of HBTU was dissolved in 50 mL of DMF and 25 mL of chloroform while stirring. 0.362 mL (2.6 mmol) of triethylamine was added and the reaction was allowed to stir for ˜3 hours. The reaction was gravity filtered into 500 mL of diethyl ether and placed at 4° C. for ˜20 hours. The precipitate was suction filtered and dried under vacuum for 5 days (LN011061). The intermediate was called MPEG5k-(AFA). 5.00 g of MPEG5k-(AFA) was dissolved in 50 mL of anhydrous DMF and 25 mL of chloroform. Argon was bubbled through the reaction for 30 minutes. 2.7 mL of piperidine was added to the reaction with argon bubbling through. The reaction was stirred for 30 minutes. The reaction was poured into 300 mL of a 1:1 MTBE:Heptane mix and placed at −15° C. for ˜23 hours. The precipitate was dried under vacuum for ˜3 hours. The polymer was dissolved in 100 mL of nanopure water with 0.100 mL of concentrated HCl. The polymer solution was poured into 2000 MWCO dialysis tubing and dialyzed against 1.5 L of nanopure water containing 0.150 mL of concentrated HCl. The dialysate was changed 8 times over the next 24 hours. The dialysate was changed to nanopure water (1.5 L) and changed 4 times over the next 21 hours. The polymer solution was suction filtered, frozen and placed on a lyophilizer. 3.75 g of material was obtained (LN011070). ¹H NMR (400 MHz, D2O/TMS): δ 7.28 (d, 1H, —C₆H₃—CH═CH—), 7.1 (s, 1H, —C₆H₃—), 7.00 (d, 1H, —C₆H₃—), 6.76 (d, 1H, —C₆H₃—), 6.36 (d, 1H, —C₆H₃—CH═CH—), 3.8-3.2 (m, 458H, PEG, —C₆H₃—OCH₃).

### Example 21

### Synthesis of Surphys-071 (MPEG5k-(VA))

4.98 g (1 mmol) of MPEG5k-(NH₂), 0.347 g (1.6 mmol) of acetyl vanillic acid and 0.617 g (1.6 mmol) of HBTU was dissolved in 50 mL of DMF and 25 mL of chloroform while stirring. 0.362 mL (2.6 mmol) of triethylamine was added and the reaction was allowed to stir for ˜3 hours. The reaction was gravity filtered into 500 mL of diethyl ether and placed at 4° C. for ˜20 hours. The precipitate was suction filtered and dried under vacuum for 5 days (LN011063). The intermediate was called MPEG5k-(AVA). 5.03 g of MPEG5k-(AVA) was dissolved in 50 mL of anhydrous DMF and 25 mL of chloroform. Argon was bubbled through the reaction for 30 minutes. 2.7 mL of piperidine was added to the reaction with argon bubbling through. The reaction was stirred for 30 minutes. The reaction was poured into 300 mL of a 1:1 MTBE:Heptane mix and placed at −15° C. for ˜23 hours. The precipitate was dried under vacuum for ˜3 hours. The polymer was dissolved in 100 mL of nanopure water with 0.100 mL of concentrated HCl. The polymer solution was poured into 2000 MWCO dialysis tubing and dialyzed against 1.5 L of nanopure water containing 0.150 mL of concentrated HCl. The dialysate was changed 8 times over the next 24 hours. The dialysate was changed to nanopure water (1.5 L) and changed 4 times over the next 21 hours. The polymer solution was suction filtered, frozen and placed on a lyophilizer. 3.90 g of material was obtained (LN011072). ¹H NMR (400 MHz, D2O/TMS): δ 7.26 (s, 1H, —C₆H₃—), 7.19 (d, 1H, —C₆H₃—), 6.81 (d, 1H, —C₆H₃—), 3.8-3.2 (m, 458H, PEG, —C₆H₃—OCH₃).

### Example 22

### Synthesis of (PEG20k-(Boc-4A-3-ABA)₈)

21.52 g (1.076 mmol) of PEG20k-(NH₂)₈, 3.908 g (13.77 mmol) of Boc-4-amino-3-acetoxybenzoic acid and 5.223 g (13.77 mmol) of HBTU was dissolved in 215 mL of DMF and 110 mL of chloroform while stirring. 3.12 mL (22.39 mmol) of triethylamine was added and the reaction was allowed to stir for ˜2 hours. The reaction was gravity filtered into 1.7 L of diethyl ether and placed at ˜4° C. for ˜3 days. The precipitate was suction filtered and dried under vacuum for 25 hours (LN011078). The intermediate was called PEG20k-(Boc-4A-3-ABA)₈. 24.55 g of material was obtained.

### Example 23

### Synthesis of (MPEG5k-(Boc-4A-3-ABA))

6.98 g (1.4 mmol) of MPEG5k-(NH₂), 0.636 g (2.24 mmol) of Boc-4-amino-3-acetoxybenzoic acid and 0.857 g (2.24 mmol) of HBTU was dissolved in 70 mL of DMF and 40 mL of chloroform while stirring. 0.507 mL (3.64 mmol) of triethylamine was added and the reaction was allowed to stir for ˜2 hours. The reaction was gravity filtered into 700 mL of diethyl ether and placed at ˜4° C. for ˜3 days. The precipitate was suction filtered and dried under vacuum for 24 hours (LN011080). The intermediate was called MPEG5k-(Boc-4A-3-ABA). 7.22 g of material was obtained.

### Example 24

### Synthesis of Surphys-076 (MPEG5k-(4A-3-HBA))

2.39 g of Surphys-078 was dissolved in 7.5 mL chloroform. 7.5 mL of trifluoroacetic acid was added slowly to the solution and allowed to stir for 30 minutes. The reaction was poured into 400 mL of diethyl ether and the flask was washed with an additional 20 mL chloroform to remove excess polymer. The mixture was placed at 4° C. for 19 hours. The precipitate was suction filtered and placed under vacuum for 23 hours. The resulting polymer was dissolved in 80 mL of nanopure water and poured into 2000 MWCO dialysis tubing. This was placed in 1.5 L of nanopure water which was changed 2 times over 3 hours. The dialysate was changed to nanopure water, which was acidified with 0.150 mL of concentrated HCl, and changed 8 times over the next ˜44 hours. The dialysate was changed to nanopure water (1.5 L) and changed 4 times over the next 3 hours. The solution was frozen and placed on a lyophilizer. 1.81 g of material was obtained (LN011409). ¹H NMR (400 MHz, D2O/TMS): δ 7.19 (d, 1H, —C₆H₃—), 7.17 (s, 1H, —C₆H₃—), 6.85 (d, 1H, —C₆H₃—), 3.8-3.2 (m, 455H, PEG).

### Example 25

### Synthesis of Surphys-077 (PEG20k-(4A-3-HBA)₈)

11.03 g of Surphys-079 was dissolved in 22 mL chloroform. 22 mL of trifluoroacetic acid was added slowly to the solution and allowed to stir for 30 minutes. The reaction was poured into 900 mL of diethyl ether and the flask was washed with an additional 20 mL of chloroform to remove excess polymer. The mixture was placed at 4° C. for 18 hours. The precipitate was suction filtered and placed under vacuum for 4 hours. The resulting polymer was dissolved in 250 mL of nanopure water and poured into 2000 MWCO dialysis tubing. This was placed in 2 L of nanopure water which was changed 2 times over 3 hours. The dialysate was changed to nanopure water, which was acidified with 0.200 mL of concentrated HCl, and changed 8 times over the next ˜40 hours. The dialysate was changed to nanopure water (2 L) and changed 4 times over the next 3 hours. The solution was frozen and placed on a lyophilizer. 9.19 g of material was obtained (LN011412). ¹H NMR (400 MHz, D2O/TMS): δ 7.19 (d, 1H, —C₆H₃—), 7.17 (s, 1H, —C₆H₃—), 6.85 (d, 1H, —C₆H₃—), 3.8-3.2 (m, 226H, PEG).

### Example 26

### Synthesis of Surphys-078 (MPEG5k-(Boc-4A-3-HBA))

4.63 g of MPEG5k-(Boc-4A-3-ABA) was dissolved in 50 mL of anhydrous DMF and 15 mL of chloroform. Argon was bubbled through the reaction for ˜40 minutes. 3 mL of piperidine was added to the reaction and was allowed to stir for 30 minutes (with argon bubbling through reaction). The reaction was poured into 300 mL of a 1:1 MTBE:Heptane mix containing 20 mL of chloroform and placed at 4° C. for ˜15 hours. The precipitate was suction filtered and placed under vacuum for 5 hours. The resulting polymer was dissolved in 100 mL of nanopure water acidified with 0.100 mL of concentrated HCl and poured into 2000 MWCO dialysis tubing. This was placed in 1.5 L of nanopure water acidified with concentrated HCl (0.150 mL). The dialysate was changed 9 times over the next ˜42 hours. The dialysate was changed to nanopure water (1.5 L) and changed 4 times over the next 4 hours. The solution was suction filtered, frozen and placed on a lyophilizer. 3.6 g of material was obtained (LN011093). ¹H NMR (400 MHz, D2O/TMS): δ 7.66 (d, 1H, —C₆H₃—), 7.26 (d, 1H, —C₆H₃—), 7.23 (s, 1H, —C₆H₃—), 3.8-3.2 (m, 455H, PEG), 1.41 (s, 9H, —NH—COOC(CH₃)₃—).

### Example 27

### Synthesis of Surphys-079 (PEG20k-(Boc-4A-3-HBA)₈)

18.0 g of PEG20k-(Boc-4A-3-ABA)₈ was dissolved in 150 mL of anhydrous DMF. Argon was bubbled through the reaction for ˜50 minutes. 10 mL of piperidine was added to the reaction and was allowed to stir for 30 minutes (with argon bubbling through reaction). The reaction was poured into 1175 mL of a 2:15:15 chloroform:MTBE:Heptane mix and placed at 4° C. for ˜15 hours. The precipitate was suction filtered and placed under vacuum for 5 hours.

The resulting polymer was dissolved in 400 mL of nanopure water acidified with 0.400 mL concentrated HCl and poured into 2000 MWCO dialysis tubing. This was placed in 3 L of nanopure water acidified with concentrated HCl (0.300 mL). The dialysate was changed 9 times over the next ˜43 hours. The dialysate was changed to nanopure water (3 L) and changed 4 times over the next 4 hours. The solution was suction filtered, frozen and placed on a lyophilizer. 15.01 g of material was obtained (LN011086). ¹H NMR (400 MHz, D2O/TMS): δ 7.66 (d, 1H, —C₆H₃—), 7.26 (d, 1H, —C₆H₃—), 7.23 (s, 1H, —C₆H₃—), 3.8-3.2 (m, 226H, PEG), 1.41 (s, 9H, —NH—COOC(CH₃)₃—).

### Example 28

### Synthesis of Surphys-080 (MPEG5k-(4A-3-ABA))

2.55 g of MPEG5k-(Boc-4A-3-ABA) was dissolved in 5.1 mL of chloroform. 5.1 mL of trifluoroacetic acid was slowly added to the solution and allowed to stir for 30 minutes. The solution was then poured into 200 mL of diethyl ether (flask was washed with 5 mL chloroform which was poured into diethyl ether solution) and placed at 4° C. for 19 hours. The precipitate was suction filtered and transferred to a beaker. This was placed under vacuum for ˜6 hours, then dissolved in 50 mL of nanopure water. The solution was poured into 2000MWCO dialysis tubing, and placed in 1.5 L of nanopure water. The dialysate was changed twice over a period of 2 hours. The dialysate was changed to nanopure water (1.5 L) acidified with concentrated HCl (0.150 mL). The dialysate was changed 7 times over the next ˜40 hours. The dialysate was changed to nanopure water (1.5 L) and changed 4 times over the next 3 hours. The solution was suction filtered, frozen and placed on a lyophilizer. 2.20 g of material was obtained (LN011090). The amine was not fully deprotected of the Boc protecting group so the polymer was dissolved in 10 mL of chloroform followed by the addition of 10 mL of trifluoroacetic acid. The reaction was allowed to stir for 30 minutes. The reaction was poured into 300 mL of diethyl ether (the flask was washed with 10 mL of chloroform and poured into diethyl ether as well). The solution was placed at 4° C. for ˜16 hours. The precipitate was suction filtered and placed under vacuum for ˜23 hours. The polymer was dissolved in 40 mL of nanopure water. The solution was poured into 2000MWCO dialysis tubing, and placed in 1 L of nanopure water. The dialysate was changed twice over a period of 3 hours. The dialysate was changed to nanopure water (1 L) acidified with concentrated HCl (0.100 mL). The dialysate was changed 7 times over the next 44 hours. The dialysate was changed to nanopure water (1 L) and changed 4 times over the next 4 hours. The solution was suction filtered, frozen and placed on a lyophilizer. 1.23 g of material was obtained (LN011421). ¹H NMR (400 MHz, D2O/TMS): δ 7.59 (d, 1H, —C₆H₃—), 7.25 (s, 1H, —C₆H₃—), 7.22 (d, 1H, —C₆H₃—), 3.8-3.2 (m, 455H, PEG), 2.13 (s, 3H, —OOCCH₃—).

### Example 29

### Synthesis of Surphys-081 (PEG20k-(4A-3-ABA)₈)

6.5 g of PEG20k-(Boc-4A-3-ABA)₈ was dissolved in 15 mL of chloroform. 15 mL of trifluoroacetic acid was slowly added to the solution and allowed to stir for 30 minutes. The solution was then poured into 400 mL of diethyl ether and placed at 4° C. for 20 hours. 200 mL of diethyl ether was added and the solution was placed at −15° C. for ˜90 minutes. The precipitate was suction filtered and transferred to a beaker. This was placed under vacuum for ˜4 hours, then dissolved in 120 mL of nanopure water. The solution was poured into 2000MWCO dialysis tubing, and placed in 1.5 L of nanopure water. The dialysate was changed twice over a period of 3 hours. The dialysate was changed to nanopure water (1.5 L) acidified with concentrated HCl (0.150 mL). The dialysate was changed 7 times over the next 40 hours. The dialysate was changed to nanopure water (1.5 L) and changed 4 times over the next 3 hours. The solution was suction filtered, frozen and placed on a lyophilizer. 4.78 g of material was obtained (LN011082). The amine was not fully deprotected of the Boc protecting group so the polymer was dissolved in 10 mL of chloroform followed by the addition of 10 mL of trifluoroacetic acid. The reaction was allowed to stir for 30 minutes. The reaction was poured into 400 mL of diethyl ether (the flask was washed with 10 mL of chloroform and poured into diethyl ether as well). The solution was placed at 4° C. for 16 hours. The precipitate was suction filtered and placed under vacuum for ˜4 hours. The polymer was dissolved in 120 mL of nanopure water. The solution was poured into 2000MWCO dialysis tubing, and placed in 1 L of nanopure water. The dialysate was changed twice over a period of 3 hours. The dialysate was changed to nanopure water (1 L) acidified with concentrated HCl (0.100 mL). The dialysate was changed 7 times over the next 40 hours. The dialysate was changed to nanopure water (1 L) and changed 4 times over the next 4 hours. The solution was suction filtered, frozen and placed on a lyophilizer. 3.93 g of material was obtained (LN011422). ¹H NMR (400 MHz, D2O/TMS): δ 7.59 (d, 1H, —C₆H₃—), 7.25 (s, 1H, —C₆H₃—), 7.22 (d, 1H, —C₆H₃—), 3.8-3.2 (m, 226H, PEG), 2.13 (s, 3H, —OOCCH₃—).

### Example 30

### Synthesis of Surphys-082 (MPEG5k-(4H-3NPAA))

2.617 g (0.523 mmol) of MPEG5k-(NH₂), 0.209 g (0.837 mmol) of 4-Acetoxy-3-nitrophenylacetic acid and 0.325 g (0.837 mmol) of HBTU was dissolved in 26 mL of DMF and 13 mL of chloroform while stirring. 0.189 mL (1.36 mmol) of triethylamine was added and the reaction was allowed to stir for ˜90 minutes. The reaction was gravity filtered into 400 mL of diethyl ether and placed at 4° C. for ˜2 days. The precipitate was suction filtered and dried under vacuum for 24 hours (LN011405). 2.60 g of the intermediate was obtained and called MPEG5k-(4A-3NPAA). 2.60 g of MPEG5k-(4A-3NPAA) was dissolved in 20 mL DMF and argon was bubbled through the reaction for 30 minutes. 2 mL of piperidine was added to the reaction with argon bubbling through. The reaction was stirred for 30 minutes. The reaction was gravity filtered into 160 mL of a 1:1 MTBE:Heptane mix containing 10 mL of chloroform and placed at 4° C. for 20 hours. The precipitate was dried under vacuum for ˜4 hours. The polymer was dissolved in 50 mL of nanopure water with 0.050 mL of concentrated HCl. The polymer solution was poured into 2000 MWCO dialysis tubing and dialyzed against 1.0 L of nanopure water containing 0.100 mL of concentrated HCl. The dialysate was changed 8 times over the next 44 hours. The dialysate was changed to nanopure water (1.0 L) and changed 4 times over the next 3 hours. The polymer solution was suction filtered, frozen and placed on a lyophilizer. 1.97 g of material was obtained (LN011418). ¹H NMR (400 MHz, D2O/TMS): δ 7.90 (s, 1H, —C₆H₃—), 7.43 (d, 1H, —C₆H₃—), 7.01 (d, 1H, —C₆H₃—), 3.8-3.3 (m, 455H, PEG), 3.25 (s, 2H, —CH₂—COOH—).

### Example 31

### Synthesis of Surphys-083 (PEG20k-(4H-3NPAA)₈)

6.5 g (0.325 mmol) of PEG20k-(NH₂)₈, 0.997 g (4.16 mmol) of 4-Acetoxy-3-nitrophenylacetic acid and 1.592 g (4.16 mmol) of HBTU was dissolved in 65 mL of DMF and 33 mL of chloroform while stirring. 0.94 mL (6.76 mmol) of triethylamine was added and the reaction was allowed to stir for ˜90 minutes. The reaction was gravity filtered into 700 mL of diethyl ether and placed at 4° C. for ˜2 days. The precipitate was suction filtered and dried under vacuum for 24 hours (LN011407). 7.18 g of the intermediate was obtained and called PEG20k-(4A-3NPAA)₈. 7.18 g of PEG20k-(4A-3NPAA)₈ was dissolved in 60 mL DMF and argon was bubbled through the reaction for 30 minutes. 5 mL of piperidine was added to the reaction with argon bubbling through. The reaction was stirred for 30 minutes. The reaction was gravity filtered into 440 mL of a 1:1 MTBE:Heptane mix containing 30 mL of chloroform and placed at 4° C. for 20 hours. The precipitate was dried under vacuum for ˜4 hours. The polymer was dissolved in 150 mL of nanopure water with 0.150 mL of concentrated HCl. The polymer solution was poured into 2000 MWCO dialysis tubing and dialyzed against 1.5 L of nanopure water containing 0.150 mL of concentrated HCl. The dialysate was changed 10 times over the next 44 hours. The dialysate was changed to nanopure water (1.5 L) and changed 4 times over the next 3 hours. The polymer solution was suction filtered, frozen and placed on a lyophilizer. 5.72 g of material was obtained (LN011415). ¹H NMR (400 MHz, D2O/TMS): δ 7.90 (s, 1H, —C₆H₃—), 7.43 (d, 1H, —C₆H₃—), 7.01 (d, 1H, —C₆H₃—), 3.8-3.3 (m, 226H, PEG), 3.25 (s, 2H, —CH₂—COOH—).

### Example 32

### Synthesis of (MPEG5k-(Boc-3A-4ABA))

7.44 g (1.49 mmol) of MPEG5k-(NH₂), 0.6858 g (2.38 mmol) of Boc-3-amino-4-acetoxybenzoic acid and 0.9176 g (2.38 mmol) of HBTU was dissolved in 75 mL of DMF and 40 mL of chloroform while stirring. 0.543 mL (3.87 mmol) of triethylamine was added and the reaction was allowed to stir for ˜2 hours. The reaction was gravity filtered into 750 mL of diethyl ether and placed at ˜4° C. for ˜19 hours. The precipitate was suction filtered and dried under vacuum for ˜30 hours. ˜7.48 g of material was obtained (LN011430).

### Example 33

### Synthesis of (PEG20k-(Boc-3A-4ABA)₈)

24.95 g (1.25 mmol) of PEG20k-(NH₂)₈, 4.585 g (16 mmol) of Boc-3-amino-4-acetoxybenzoic acid and 6.095 g (16 mmol) of HBTU was dissolved in 250 mL of DMF and 125 mL of chloroform while stirring. 3.625 mL (26 mmol) of triethylamine was added and the reaction was allowed to stir for ˜2 hours. The reaction was gravity filtered into 2 L of diethyl ether and placed at ˜4° C. for ˜19 hours. The precipitate was suction filtered and dried under vacuum for ˜30 hours. ˜28.42 g of material was obtained (LN011432).

### Example 34

### Synthesis of Surphys-084 (MPEG5k-(3A-4ABA))

1.68 g of MPEG5k-(Boc-3A-4ABA) was dissolved in 10 mL of chloroform. 10 mL of trifluoroacetic acid was slowly added to the solution and allowed to stir for ˜40 minutes. The solution was then poured into 300 mL of diethyl ether and placed at 4° C. for 20 hours. 200 mL of heptane was added and the solution was placed back at 4° C. for another 20 hours. The precipitate was suction filtered and transferred to a beaker. This was placed under vacuum for ˜5 hours, then dissolved in 50 mL of nanopure water. The solution was poured into 2000MWCO dialysis tubing, and placed in 1.0 L of nanopure water. The dialysate was changed twice over a period of 3 hours. The dialysate was changed to nanopure water (1.0 L) acidified with concentrated HCl (0.100 mL). The dialysate was changed 5 times over the next ˜40 hours. The dialysate was changed to nanopure water (1.0 L) and changed 4 times over the next 3 hours. The solution was suction filtered, frozen and placed on a lyophilizer until dry (LN011448). The yield was not recorded. ¹H NMR (400 MHz, D2O/TMS): δ 7.8 (s, 1H, —C₆H₃—), 7.5 (d, 1H, —C₆H₃—), 6.95 (s, 1H, —C₆H₃—), 3.8-3.2 (m, 455H, PEG), 2.11 (s, 3H, CH₃—COO—C₆H₃—).

### Example 35

### Synthesis of Surphys-085 (PEG20k-(3A-4ABA)₈)

8.24 g of PEG20k-(Boc-3A-4ABA)₈ was dissolved in 25 mL of chloroform. 25 mL of trifluoroacetic acid was slowly added to the solution and allowed to stir for ˜35 minutes. The solution was then poured into 900 mL of diethyl ether and placed at 4° C. for 20 hours. 700 mL of heptane was added and the solution was placed back at 4° C. for another 20 hours. The precipitate was suction filtered and transferred to a beaker. This was placed under vacuum for ˜5 hours, then dissolved in 120 mL of nanopure water. The solution was poured into 2000MWCO dialysis tubing, and placed in 2.0 L of nanopure water. The dialysate was changed twice over a period of 3 hours. The dialysate was changed to nanopure water (2.0 L) acidified with concentrated HCl (0.200 mL). The dialysate was changed 5 times over the next ˜40 hours. The dialysate was changed to nanopure water (2.0 L) and changed 4 times over the next 3 hours. The solution was suction filtered, frozen and placed on a lyophilizer until dry (LN011445). The yield was not recorded. ¹H NMR (400 MHz, D2O/TMS): δ 7.8 (s, 1H, —C₆H₃—), 7.5 (d, 1H, —C₆H₃—), 6.95 (s, 1H, —C₆H₃—), 3.8-3.2 (m, 226H, PEG), 2.11 (s, 3H, CH₃—COO—C₆H₃—).

### Example 36

### Synthesis of Surphys-086 (MPEG5k-(Boc-3A-4HBA))

5.8 g of MPEG5k-(Boc-3A-4ABA) was dissolved in ˜50 mL of anhydrous DMF and 25 mL of chloroform. Argon was bubbled through the reaction for ˜45 minutes. 7 mL of piperidine was added to the reaction with argon bubbling through. The reaction was stirred for 30 minutes. The reaction was gravity filtered into 400 mL of a 1:1 MTBE:Heptane mix and placed at 4° C. for 20 hours. The precipitate was dried under vacuum for ˜22 hours. The polymer was dissolved in 120 mL of nanopure water with 0.120 mL of concentrated HCl. The polymer solution was poured into 2000 MWCO dialysis tubing and dialyzed against 2.0 L of nanopure water containing 0.200 mL of concentrated HCl. The dialysate was changed 9 times over the next 47 hours. The dialysate was changed to nanopure water (1.0 L) and changed 4 times over the next 3 hours. The polymer solution was suction filtered, frozen and placed on a lyophilizer. 4.82 g of material was obtained (LN011442). ¹H NMR (400 MHz, D2O/TMS): δ 7.9 (s, 1H, —C₆H₃—), 7.4 (d, 1H, —C₆H₃—), 6.9 (s, 1H, —C₆H₃—), 3.8-3.2 (m, 455H, PEG), 1.41 (s, 9H, —NH—COOC(CH₃)₃).

### Example 37

### Synthesis of Surphys-087 (PEG20k-(Boc-3A-4HBA)₈)

20 g of PEG20k-(Boc-3A-4ABA)₈ was dissolved in ˜160 mL of anhydrous DMF. Argon was bubbled through the reaction for ˜55 minutes. 15 mL of piperidine was added to the reaction with argon bubbling through. The reaction was stirred for 30 minutes. The reaction was gravity filtered into 1200 mL of a 1:1 MTBE:Heptane mix containing 160 mL of chloroform and placed at 4° C. for 20 hours. The precipitate was dried under vacuum for ˜22 hours. The polymer was dissolved in 360 mL of nanopure water with 0.360 mL of concentrated HCl. The polymer solution was poured into 2000 MWCO dialysis tubing and dialyzed against 4.0 L of nanopure water containing 0.400 mL of concentrated HCl. The dialysate was changed 9 times over the next 47 hours. The dialysate was changed to nanopure water (4.0 L) and changed 4 times over the next 3 hours. The polymer solution was suction filtered, frozen and placed on a lyophilizer. 16.4 g of material was obtained (LN011439). ¹H NMR (400 MHz, D2O/TMS): δ 7.9 (s, 1H, —C₆H₃—), 7.4 (d, 1H, —C₆H₃—), 6.9 (s, 1H, —C₆H₃—), 3.8-3.2 (m, 226H, PEG), 1.41 (s, 9H, —NH—COOC(CH₃)₃).

### Example 38

### Synthesis of Surphys-088 (MPEG5k-(3A-4HBA))

3.1 g of MPEG5k-(Boc-3A-4HBA) was dissolved in 12 mL of chloroform. 12 mL of trifluoroacetic acid was slowly added to the solution and allowed to stir for ˜30 minutes. The solution was then poured into 400 mL of a 1:1 MTBE:Heptane mix and placed at 4° C. for ˜3 days. The precipitate was suction filtered and transferred to a beaker. This was placed under vacuum for ˜24 hours, then dissolved in 100 mL of nanopure water. The solution was poured into 2000MWCO dialysis tubing, and placed in 1.5 L of nanopure water. The dialysate was changed twice over a period of 4 hours. The dialysate was changed to nanopure water (1.5 L) acidified with concentrated HCl (0.150 mL). The dialysate was changed 5 times over the next ˜40 hours. The dialysate was changed to nanopure water (1.5 L) and changed 4 times over the next 3 hours. The solution was suction filtered, frozen and placed on a lyophilizer until dry. 2.36 g of material was obtained (LN011472). ¹H NMR (400 MHz, D2O/TMS): δ 7.36 (s, 1H, —C₆H₃—), 7.7.31 (d, 1H, —C₆H₃—), 6.88 (d, 1H, —C₆H₃—), 3.8-3.2 (m, 455H, PEG).

### Example 39

### Synthesis of Surphys-089 (PEG20k-(3A-4HBA)₈)

12.05 g of PEG20k-(Boc-3A-4HBA)₈ was dissolved in 35 mL of chloroform. 35 mL of trifluoroacetic acid was slowly added to the solution and allowed to stir for ˜30 minutes. The solution was then poured into 1200 mL of a 1:1 MTBE:Heptane mix and placed at 4° C. for ˜3 days. The precipitate was suction filtered and transferred to a beaker. This was placed under vacuum for ˜24 hours, then dissolved in 250 mL of nanopure water. The solution was poured into 2000MWCO dialysis tubing, and placed in 3.0 L of nanopure water. The dialysate was changed twice over a period of 4 hours. The dialysate was changed to nanopure water (3.0 L) acidified with concentrated HCl (0.300 mL). The dialysate was changed 5 times over the next ˜40 hours. The dialysate was changed to nanopure water (3.0 L) and changed 4 times over the next 3 hours. The solution was suction filtered, frozen and placed on a lyophilizer until dry. 9.55 g of material was obtained (LN011469). ¹H NMR (400 MHz, D2O/TMS): δ 7.36 (s, 1H, —C₆H₃—), 7.31 (d, 1H, —C₆H₃—), 6.88 (d, 1H, —C₆H₃—), 3.8-3.2 (m, 226H, PEG).

### Example 40

### Synthesis of Surphys-090 (MPEG5k-(CA))

4.98 g (1 mmol) of MPEG5k-(NH₂), 0.433 g (1.6 mmol) of 3,4-diacetoxycaffeic acid and 0.6115 g (1.6 mmol) of HBTU was dissolved in 50 mL of DMF and 25 mL of chloroform while stirring. 0.362 mL (2.6 mmol) of triethylamine was added and the reaction was allowed to stir for ˜2 hours. The reaction was gravity filtered into 500 mL of diethyl ether and placed at 4° C. for ˜18 hours. The precipitate was suction filtered and dried under vacuum for 30 hours (LN011434). The intermediate was called MPEG5k-(3,4-DACA). 4.81 g of MPEG5k-(3,4-DACA) was dissolved in 30 mL of anhydrous DMF. Argon was bubbled through the reaction for 30 minutes. 2.4 mL of piperidine was added to the reaction with argon bubbling through. The reaction was stirred for 30 minutes. The reaction was poured into 300 mL of a 1:1 MTBE:Heptane mix containing 20 mL of chloroform and placed at 4° C. for ˜20 hours. The precipitate was dried under vacuum for ˜29 hours. The polymer was dissolved in 100 mL of nanopure water with 0.100 mL of concentrated HCl. The polymer solution was poured into 2000 MWCO dialysis tubing and dialyzed against 1.0 L of nanopure water containing 0.100 mL of concentrated HCl. The dialysate was changed 8 times over the next 43 hours. The dialysate was changed to nanopure water (1.0 L) and changed 4 times over the next 3 hours. The polymer solution was suction filtered, frozen and placed on a lyophilizer until dry (LN011454). The yield was not recorded. ¹H NMR (400 MHz, D2O/TMS): δ 7.32 (d, 1H, —C₆H₃—CH═CH—), 7.07 (s, 1H, —C₆H₃—), 7.0 (d, 1H, —C₆H₃—), 6.83 (d, 1H, —C₆H₃—), 6.39 (d, 1H, —C₆H₃—CH═CH—), 3.7-3.4 (m, 455H, PEG).

### Example 41

### Synthesis of Surphys-091 (MPEG5k-(GA))

5.03 g (1 mmol) of MPEG5k-(NH₂), 0.482 g (1.6 mmol) of 3,4,5-triacetoxybenzoic acid and 0.612 g (1.6 mmol) of HBTU was dissolved in 50 mL of DMF and 30 mL of chloroform while stirring. 0.362 mL (2.6 mmol) of triethylamine was added and the reaction was allowed to stir for ˜2 hours. The reaction was gravity filtered into 400 mL of diethyl ether and placed at 4° C. for ˜1 hour. The precipitate was suction filtered and dried under vacuum for 20 hours (LN011461). The intermediate was called MPEG5k-(3,4,5-TABA). 5.17 g of MPEG5k-(3,4,5-TABA) was dissolved in 30 mL of anhydrous DMF and 21 mL of chloroform. Argon was bubbled through the reaction for 30 minutes. 4.5 mL of piperidine was added to the reaction with argon bubbling through. The reaction was stirred for 30 minutes. The reaction was poured into 400 mL of a 1:1 MTBE:Heptane mix and placed at 4° C. for ˜3 days. The precipitate was dried under vacuum for ˜23 hours. The polymer was dissolved in 125 mL of nanopure water with 0.125 mL of concentrated HCl. The polymer solution was poured into 2000 MWCO dialysis tubing and dialyzed against 1.5 L of nanopure water containing 0.150 mL of concentrated HCl. The dialysate was changed 8 times over the next 44 hours. The dialysate was changed to nanopure water (1.5 L) and changed 4 times over the next 3 hours. The polymer solution was suction filtered, frozen and placed on a lyophilizer until dry (LN011466). ¹H NMR (400 MHz, D2O/TMS): δ 6.84 (s, 2H, —C₆H₃—), 3.8-3.2 (m, 455H, PEG).

### Example 42

### Synthesis of Medhesive-077 (PEG20k-(GA)₈)

17 g (0.85 mmol) of PEG20k-(NH₂)₈, 3.273 g (10.88 mmol) of 3,4,5-triacetoxybenzoic acid and 4.16 g (10.88 mmol) of HBTU was dissolved in 170 mL of DMF and 90 mL of chloroform while stirring. 2.465 mL (17.68 mmol) of triethylamine was added and the reaction was allowed to stir for ˜2 hours. The reaction was gravity filtered into 1400 mL of diethyl ether and placed at 4° C. for ˜17 hours. The precipitate was suction filtered and dried under vacuum for 26 hours (LN011459). The intermediate was called PEG20k-(3,4,5-TABA)₈. 19.55 g of PEG20k-(3,4,5-TABA)₈ was dissolved in 160 mL of anhydrous DMF. Argon was bubbled through the reaction for 30 minutes. 15 mL of piperidine was added to the reaction with argon bubbling through. The reaction was stirred for 30 minutes. The reaction was poured into 1200 mL of a 1:1 MTBE:Heptane mix containing 80 mL of chloroform and placed at 4° C. for ˜3 days. The precipitate was suction filtered and dried under vacuum for ˜23 hours. The polymer was dissolved in 375 mL of nanopure water with 0.375 mL of concentrated HCl. The polymer solution was poured into 2000 MWCO dialysis tubing and dialyzed against 3.0 L of nanopure water containing 0.300 mL of concentrated HCl. The dialysate was changed 8 times over the next 44 hours. The dialysate was changed to nanopure water (3.0 L) and changed 4 times over the next 3 hours. The polymer solution was suction filtered, frozen and placed on a lyophilizer until dry. 15.56 g of polymer was obtained (LN011463). ¹H NMR (400 MHz, D2O/TMS): δ 6.84 (s, 2H, —C₆H₃—), 3.8-3.2 (m, 226H, PEG).

### Example 43

### Synthesis of Medhesive-079 (PEG20k-(CA)₈)

14.97 g (0.75 mmol) of PEG20k-(NH₂)₈, 2.61 g (9.6 mmol) of 3,4-Diacetoxycaffeic Acid and 3.66 g (9.6 mmol) of HBTU was dissolved in 150 mL of DMF and 75 mL of chloroform while stirring. 2.175 mL (15.6 mmol) of triethylamine was added and the reaction was allowed to stir for ˜2 hours. The reaction was gravity filtered into 1400 mL of diethyl ether and placed at 4° C. for ˜18 hours. The precipitate was suction filtered and dried under vacuum for 30 hours (LN011436). The intermediate was called PEG20k-(3,4-DACA)₈. 16.7 g of PEG20k-(3,4-DA CA)₈ was dissolved in 100 mL of anhydrous DMF and 60 mL of chloroform. Argon was bubbled through the reaction for 40 minutes. 12 mL of piperidine was added to the reaction with argon bubbling through. The reaction was stirred for 30 minutes. The reaction was poured into 1000 mL of a 1:1 MTBE:Heptane mix and placed at 4° C. for ˜20 hours. The precipitate was suction filtered and dried under vacuum for ˜28 hours. The polymer was dissolved in 310 mL of nanopure water with 0.310 mL of concentrated HCl. The polymer solution was poured into 2000 MWCO dialysis tubing and dialyzed against 3.0 L of nanopure water containing 0.300 mL of concentrated HCl. The dialysate was changed 8 times over the next 44 hours. The dialysate was changed to nanopure water (3.0 L) and changed 4 times over the next 3 hours. The polymer solution was suction filtered, frozen and placed on a lyophilizer until dry. (LN011451). The yield was not recorded. ¹H NMR (400 MHz, D2O/TMS): δ 7.31 (d, 1H, —C₆H₃—CH═CH—), 7.06 (s, 1H, —C₆H₃—), 7.00 (d, 1H, —C₆H₃—), 6.83 (d, 1H, —C₆H₃—), 6.38 (d, 1H, d, 1H, —C₆H₃—CH═CH—), 3.8-3.2 (m, 226H, PEG).

### Example 44

### Synthesis of PEG10k-(ADA)₄

50 g (5 mmol) of PEG10k-(OH)₄ was dissolved in 125 mL of chloroform while stirring under argon in a water bath at room temperature. 18.09 g (60 mmol) of Boc-11-aminoundecanoic acid was added to the PEG solution. When the mixture was fully dissolved, 12.40 g (60 mmol) of DCC in 100 mL of chloroform was added to the mixture along with 0.5004 g (4 mmol) of DMAP. The reaction was stirred under argon for ˜24 hours. The insoluble urea was suction filtered off. The mixture was placed in a round bottom flask under argon. 215 mL of 4M HCl in Dioxane was added to the mixture and stirred under argon for 30 minutes. The solvent was roto evaporated off. The resulting polymer was dissolved in 1 L of nanopure water and placed in 2000 MWCO dialysis tubing. This was dialyzed against 7 L of nanopurewater. The dialysate was changed 6 times over 21 hours. The polymer solution was suction filtered, frozen and placed on a lyophilizer until dry. 41.33 g of material was obtained (LN012111). ¹H NMR (400 MHz, DMSO/TMS): δ 7.79 (s, 2H, —OOC(CH₂)₁₀—NH), 4.11 (t, 2H, —CH₂—OOC(CH₂)₁₀—), 3.8-3.2 (m, 226H, PEG), 2.74 (t, 2H, —OOCCH₂(CH₂)₉—NH₂), 2.28 (t, 2H, —OOC(CH₂)₉—CH₂—NH₂), 1.51 (m, 4H, —OOCCH₂CH₂(CH₂)₆CH₂CH₂—NH₂), 1.24 (m, 12H, —OOCCH₂CH₂(CH₂)₆CH₂CH₂—NH₂).

### Example 45

### Synthesis of PEG20k-(GABA)₄

99.99 g (5 mmol) of PEG20k-(OH)₄ was dissolved in 225 mL of chloroform while stirring under argon in a water bath at room temperature. 24.37 g (120 mmol) of Boc-gamma-aminobutyric acid was added to the PEG solution. When the mixture was fully dissolved, 24.76 g (120 mmol) of DCC in 225 mL of chloroform was added to the mixture along with 0.998 g (8 mmol) of DMAP. The reaction was stirred under argon for ˜24 hours. The insoluble urea was suction filtered off. The mixture was placed in a round bottom flask under argon. 425 mL of 4M HCl in Dioxane was added to the mixture and stirred under argon for 30 minutes. The solvent was roto evaporated off. The resulting polymer was dissolved in 2 L of nanopure water and placed in 2000 MWCO dialysis tubing. This was dialyzed against 14 L of nanopure water. The dialysate was changed 6 times over 21 hours. The polymer solution was suction filtered, frozen and placed on a lyophilizer until dry. 87.88 g of material was obtained (LN012125). ¹H NMR (400 MHz, D2O/TMS): δ 4.15 (t, 2H, PEG-O—CH₂—CH₂—OOC—), 3.8-3.2 (m, 452H, PEG), 2.91 (t, 2H, —OOC—CH₂—CH₂—CH₂—NH₂), 2.43 (t, 2H, —OOC—CH₂—CH₂—CH₂—NH₂), 1.84 (m, 2H, —OOC—CH₂—CH₂—CH₂—NH₂).

### Example 46

### Synthesis of PEG20k-(GABA)₈

49.99 g (2.55 mmol) of PEG20k-(OH)₈ was dissolved in 125 mL of chloroform while stirring under argon in a water bath at room temperature. 12.24 g (60 mmol) of Boc-gamma-aminobutyric acid was added to the PEG solution. When the mixture was fully dissolved, 12.57 g (60 mmol) of DCC in 100 mL of chloroform was added to the mixture along with 0.5177 g (4 mmol) of DMAP. The reaction was stirred under argon for ˜24 hours. The insoluble urea was suction filtered off. The mixture was placed in a round bottom flask under argon. 220 mL of 4M HCl in Dioxane was added to the mixture and stirred under argon for 45 minutes. The solvent was roto evaporated off. The resulting polymer was dissolved in 1 L of nanopure water and placed in 2000 MWCO dialysis tubing. This was dialyzed against 7 L of nanopurewater. The dialysate was changed 6 times over 21 hours. The polymer solution was suction filtered, frozen and placed on a lyophilizer until dry. 40 g of material was obtained (LN012128). ¹H NMR (400 MHz, D2O/TMS): δ 4.15 (t, 2H, PEG-O—CH₂—CH—OOC—), 3.8-3.2 (m, 226H, PEG), 2.91 (t, 2H, —OOC—CH₂—CH₂—CH₂—NH₂), 2.43 (t, 2H, —OOC—CH₂—CH₂—CH₂—NH₂), 1.84 (m, 2H, —OOC—CH₂—CH₂—CH₂—NH₂).

### Example 47

### Synthesis of PEG20k-(β-Ala)₈

100.35 g (5 mmol) of PEG20k-(OH)₈ was dissolved in 225 mL of chloroform while stirring under argon in a water bath at room temperature. 22.71 g (120 mmol) of Boc-β-Alanine was added to the PEG solution. When the mixture was fully dissolved, 24.77 g (120 mmol) of DCC in 225 mL of chloroform was added to the mixture along with 0.989 g (8 mmol) of DMAP. The reaction was stirred under argon for ˜22 hours. The insoluble urea was suction filtered off. The mixture was placed in a round bottom flask under argon. 425 mL of 4M HCl in Dioxane was added to the mixture and stirred under argon for 30 minutes. The solvent was roto-evaporated off. The resulting polymer was dissolved in 2 L of nanopure water and placed in 2000 MWCO dialysis tubing. This was dialyzed against 14 L of nanopurewater. The dialysate was changed 6 times over 23 hours. The polymer solution was suction filtered, frozen and placed on a lyophilizer until dry. 81.67 g of material was obtained (LN012420). ¹H NMR (400 MHz, DMSO/TMS): δ 4.15 (t, 2H, PEG-O—CH₂—CH₂—OOC—), 3.8-3.2 (m, 226H, PEG), 3.00 (t, 2H, —OOC—CH₂—CH₂—NH₂), 2.68 (t, 2H, —OOC—CH₂—CH₂—NH₂).

### Example 48

### Synthesis of PEG20k-(Lyse)₈

Combined 150.9 g of 8-arm PEG-OH and 300 mL of toluene in a 1 L round bottom flask equipped with a Dean-Stark apparatus, condensation column, and an Argon inlet. The mixture was stirred in a 160-165° C. oil bath until about ¾ of toluene was evaporated and collected with Argon purging. The reaction mixture was allowed to cool to room temperature and 675 mL of chloroform was added. 62.4 g of N,N′-α,ε-Bis-Boc-Lysine, 37.2 g of N,N′-dicyclohexylcarbodiimide, and 729 mg of 4-(Dimethylamino) pyridine were added and the reaction mixture was stirred in a room temperature water bath for overnight with Argon purging. Filtered the insoluble urea byproduct with coarse filter paper through vacuum filtration and filtrate was added to 3.75 L of diethyl ether for overnight at 4° C. After collecting and drying the precipitate, 159.61 g of PEG20k-(Boc₂Lyse)₈ was obtained. The polymer was dissolved in 319 mL of chloroform and 319 mL of TFA was slowly added. The mixture was stirred at room temperature for 30 min and added to 3.2 mL of diethyl ether. The mixture was placed in −20° C. for overnight and the supernatant was decanted. The gooey solid was precipitated again in chloroform/ether mixture and dried with vacuum pump. The solid was then dissolved in 2 L of deionized water and dialyzed with 3500 MWCO dialysis tubes for two hours in 20 L of deionized water followed by 40 hrs in 20 L of water acidified to pH 3.5 with HCl, and 2 hrs in deionized water. After lyophilization, 83.35 g of PEG20k-(Lyse)₈ was obtained. ¹H NMR confirmed the structure.

### Example 49

### Synthesis of PEG20k-(MGAe)₈

10 g of 8-armed PEG-OH (20,000 MW; 4 mmol —OH) was added to 2.56 g of 3-Methyl glutaric anhydride (20 mmol), 100 mL chloroform and 1.6 mL of pyridine taken in a round bottom flask equipped with a condensation column. Refluxed the mixture at 80° C. in an oil bath with Ar purging overnight. The polymer solution was cooled to room temperature, added 100 mL of chloroform. The reaction mixture was washed successively with 100 mL each of 12 mM HCl, saturated NaCl solution, and H₂O. The organic layer is then dried over MgSO₄ and filtered. Reduced the filtrate to around 100 mL and added to 900 mL of diethyl ether. Collected the precipitate via filtration and dried the precipitate. 1H NMR confirmed the structure.

### Example 50

### Synthesis of Medhesive-117 (PEG20k-(TMu)₈)

50 g (0.475 mmol) of PEG20k-(OH)₈ was azeotropically dried 2 times with 200 mL of toluene. The PEG was dried under vacuum. The PEG was dissolved in 200 mL of toluene through gentle heating with argon purging. 100 mL of phosgene solution was added. The reaction was heated at 55-65° C. for 4 hours with argon purging. The reaction was removed from the heat source and allowed to cool to room temperature with argon bubbling through the reaction to remove excess phosgene. The toluene was roto evaporated off. 200 mL of toluene was added and roto evaporated off again. The polymer was placed under vacuum overnight. 5.77 g (50 mmol) of NHS and 200 mL of chloroform was added to the reaction. 6.16 mL (44 mmol) of triethlamine was added to 50 mL of chloroform and added dropwise. The reaction was stirred with argon purging for 4 hours. 6.86 g (50 mmol) of tyramine was added to 50 mL of DMF and was added to the reaction. 7 mL of triethylamine was added to the reaction and was allowed to stir overnight. The reaction was gravity filtered into 800 mL of diethyl ether and placed at 4° C. overnight. The precipitate was suction filtered and dried under vacuum. The polymer was dissolved in 400 mL of 12 mM HCl. Insoluble material was removed through suction filtration. The polymer was placed into 3500 MWCO dialysis tubing and dialyzed against 4 L of H₂O for 24 hours. 27.2 g of product was obtained. ¹H NMR (400 MHz, CDCl₃): δ 7.00 (d, 2H, C₆H₄—), 6.95 (s, 1H, C₆H₄—), 6.62 (d, 2H, C₆H₄—), 4.20 (t, 2H, —O—CH₂—CH₂—PEG-), 3.8-3.0 (m, 228H, PEG, —CH₂—CH₂—C₆H₄—OH), 2.70 (m, 2H, NHCOO—CH₂—CH₂—).

### Example 51

### Synthesis of Medhesive-120 (PEG20k-(LysHF2)₈)

9.99 g (0.475 mmol) of PEG20k-(Lyse)₈ was dissolved in 66 mL of chloroform and 33 mL of DMF. 2.8989 g (14.78 mmol) of Hydroferulic Acid, 2.00 g (14.80 mmol) of HOBt and 5.6086 g (14.79 mmol) of HBTU was added to the reaction and stirred until completely dissolved. When the solution was clear, 2.07 mL (14.85 mmol) of triethylamine was added and the reaction was allowed to stir for ˜90 minutes. The reaction was gravity filtered into 600 mL of diethyl ether and placed at ˜4° C. for ˜24 hours. The precipitate was suction filtered and dried under vacuum for ˜15 hours. The material was dissolved in ˜100 mL of 12.1 mM HCl, gravity filtered and placed in 3500 MWCO dialysis tubing. This was dialyzed against 3.5 L of nanopure water acidified with 0.400 mL of concentrated HCl. The dialysate was changed 5 times over 24 hours. The dialysate was changed to nanopure water and changed 5 times over 24 hour. The polymer solution was gravity filtered, frozen and placed on a lyophilizer until dry. 5.90 g of material was obtained (LN006289). ¹H NMR (400 MHz, D2O/TMS): δ 6.8-6.5 (m, 6H, —C₆H₃—), 4.15 (t, 2H, —O—CH₂—CH₂—PEG-), 3.8-3.0 (m, 232H, PEG, —C₆H₃—O—CH₃), 3.0-0.5 (m, 16H, —OCOCH(NHCH₂CH₂—)CH₂CH₂CH₂CH₂—NH—CH₂CH₂—).

### Example 52

### Synthesis of Medhesive-121 (PEG20k-(MGAMTe)₈)

10 g (0.475 mmol) of PEG20k-(MGAe)₈ was dissolved in 40 mL of chloroform. 1.2312 g (6.04 mmol) of 3-Methoxytyramine Hydrochloride, 0.8128 g (6.02 mmol) of HOBt and 2.2869 g (6.03 mmol) of HBTU was dissolved in 27 mL DMF. The two solutions were added together. An additional 28 mL of DMF was added to the reaction. When the solution was clear, 1.26 mL (9.04 mmol) of triethylamine was added and the reaction was allowed to stir for ˜1 hour. The reaction was gravity filtered into 600 mL of diethyl ether and placed at ˜4° C. for ˜24 hours. The precipitate was suction filtered and dried under vacuum for ˜17 hours. The material was dissolved in ˜100 mL of 12.1 mM HCl, gravity filtered and placed in 3500 MWCO dialysis tubing. This was dialyzed against 3.5 L of nanopure water acidified with 0.400 mL of concentrated HCl. The dialysate was changed 13 times over 48 hours. The dialysate was changed to nanopure water and changed once over 1 hour. The polymer solution was gravity filtered, frozen and placed on a lyophilizer until dry. 8.11 g of material was obtained (LN006501). ¹H NMR (400 MHz, D₂O): δ 6.81-6.60 (m, 3H, C₆H₃—), 4.13 (t, 2H, —O—CH₂—CH₂—PEG-), 3.8-3.0 (m, 231H, PEG, —CH₂—CH₂—C₆H₃—O—CH₃), 2.65 (m, 2H, NHCO—CH₂—CH₂—), 2.07-1.90 (m, 5H, —OOC—CH₂CH(CH₃)CH₂—), 0.71 (d, 3H, —OOC—CH₂CH(CH₃)CH₂—).

### Example 53

### Synthesis of Medhesive-122 (PEG20k-(MGAMTe)₈)

15 g (0.713 mmol) of PEG20k-(MGAe)₈ was dissolved in 60 mL of chloroform. 1.72 g (9.07 mmol) of vanillylamine hydrochloride, 1.2184 g (9.02 mmol) of HOBt and 3.4301 g (9.04 mmol) of HBTU was dissolved in 40 mL DMF. The two solutions were added together. An additional 40 mL of DMF was added to the reaction. When the solution was clear, 1.89 mL (13.56 mmol) of triethylamine was added and the reaction was allowed to stir for ˜1 hour. The reaction was gravity filtered into 900 mL of diethyl ether and placed at ˜4° C. for ˜19 hours. The precipitate was suction filtered and dried under vacuum for ˜12 hours. The material was dissolved in ˜150 mL of 12.1 mM HCl, gravity filtered and placed in 3500 MWCO dialysis tubing. This was dialyzed against 3.5 L of nanopure water acidified with 0.400 mL of concentrated HCl. The dialysate was changed 13 times over 48 hours. The dialysate was changed to nanopure water and changed once over 1 hour. The polymer solution was gravity filtered, frozen and placed on a lyophilizer until dry. 11.90 g of material was obtained (LN006516). ¹H NMR (400 MHz, D₂O): δ 6.85-6.65 (m, 3H, C₆H₃—), 4.17 (t, 2H, —O—CH—CH₂—PEG-), 3.8-3.0 (m, 231H, PEG, —CH₂—C₆H₃—O—CH₃), 2.07-1.90 (m, 5H, —OOC—CH₂CH(CH₃)CH₂—), 0.71 (d, 3H, —OOC—CH₂CH(CH₃)CH₂—).

### Example 54

### Synthesis of Medhesive-123 (PEG20k-(LysHVA2)₈)

10 g (0.475 mmol) of PEG20k-(Lyse)₈ was dissolved in 65 mL of chloroform and 35 mL of DMF. 2.6913 g (14.77 mmol) of homovanillic acid. 2.005 g (14.84 mmol) of HOBt and 5.6092 g (14.79 mmol) of HBTU was added to the reaction and stirred until completely dissolved. When the solution was clear, 2.07 mL (14.85 mmol) of triethylamine was added and the reaction was allowed to stir for ˜90 minutes. The reaction was gravity filtered into 600 mL of diethyl ether and placed at ˜4° C. for ˜7 hours. The precipitate was suction filtered and dried under vacuum for ˜11 hours. The material was dissolved in ˜100 mL of 12.1 mM HCl, gravity filtered and placed in 3500 MWCO dialysis tubing. This was dialyzed against 3.5 L of nanopure water acidified with 0.400 mL of concentrated HCl. The dialysate was changed 5 times over 24 hours. The dialysate was changed to nanopure water and changed 5 times over 24 hour. The polymer solution was gravity filtered, frozen and placed on a lyophilizer until dry. The yield of material was not recorded (LN006530). ¹H NMR (400 MHz, D2O/TMS): δ 6.8-6.5 (m, 6H, —C₆H₃—), 4.12 (t, 2H, —O—CH₂—CH₂—PEG-), 3.8-3.3 (m, 232H, PEG, —C₆H₃—O—CH₃), 3.3-0.5 (m, 12H, —OCOCH(NHCH₂-)CH₂CH₂CH₂CH₂—NH—CH₂—).

### Example 55

### Synthesis of Medhesive-125 (PEG20k-(MGAHVTAe)₈)

15 g (0.713 mmol) of PEG20k-(MGAe)₈ was dissolved in 60 mL of chloroform. 1.525 mL (9.07 mmol) of Homoveratrylamine, 1.2175 g (9.02 mmol) of HOBt and 3.425 g (9.04 mmol) of HBTU was dissolved in 40 mL DMF. The two solutions were added together. An additional 40 mL of DMF was added to the reaction. When the solution was clear, 1.89 mL (13.56 mmol) of triethylamine was added and the reaction was allowed to stir for ˜1 hour. The reaction was gravity filtered into 850 mL of diethyl ether and placed at ˜4° C. for ˜16 hours. The precipitate was suction filtered and dried under vacuum for ˜4 days. The material was dissolved in ˜150 mL of 12.1 mM HCl, gravity filtered and placed in 3500 MWCO dialysis tubing. This was dialyzed against 3.5 L of nanopure water acidified with 0.400 mL of concentrated HCl. The dialysate was changed 13 times over 48 hours. The dialysate was changed to nanopure water and changed once over 1 hour. The polymer solution was gravity filtered, frozen and placed on a lyophilizer until dry. 12.80 g of material was obtained (LN006546). ¹H NMR (400 MHz, D₂O): δ 6.86-6.70 (m, 3H, C₆H₃—), 4.11 (t, 2H, —O—CH₂—CH₂—PEG-), 3.8-3.0 (m, 234H, PEG, —CH₂—CH₂—C₆H₃—(O—CH₃)₂), 2.65 (m, 2H, NHCO—CH₂—CH₂—), 2.07-1.90 (m, 5H, —OOC—CH₂CH(CH₃)CH₂—), 0.70 (d, 3H, —OOC—CH₂CH(CH₃)CH₂—).

### Example 56

### Synthesis of Medhesive-126 (PEG20k-(MGATMe)₈)

5.05 g (0.238 mmol) of PEG20k-(MGAe)₈ was dissolved in 22 mL of chloroform. 0.5756 g (4.2 mmol) of tyramine, 0.4075 g (3.02 mmol) of HOBt and 1.1425 g (3.01 mmol) of HBTU was dissolved in 14 mL DMF. The two solution were added together. An additional 14 mL of DMF was added to the reaction. When the solution was clear, 0.63 mL (4.52 mmol) of triethylamine was added and the reaction was allowed to stir for ˜1 hour. The reaction was gravity filtered into 300 mL of diethyl ether and placed at ˜4° C. for ˜18 hours. The precipitate was suction filtered and dried under vacuum for ˜23 hours. The material was dissolved in ˜50 mL of 12.1 mM HCl and placed in 3500 MWCO dialysis tubing. This was dialyzed against 3.5 L of nanopure water acidified with 0.400 mL of concentrated HCl. The dialysate was changed 13 times over 48 hours. The dialysate was changed to nanopure water and changed once over 2 hours. The polymer solution was gravity filtered, frozen and placed on a lyophilizer until dry. 3.37 g of material was obtained (LN005973). H NMR (400 MHz, D₂O): δ 7.02 (d, 2H, C₆H₄—), 6.69 (d, 2H, C₆H₄—), 4.13 (t, 2H, —O—CH—CH₂—PEG-), 3.8-3.0 (m, 228H, PEG, —CH₂—CH₂—C₆H₄), 2.65 (m, 2H, NHCO—CH₂—CH—), 2.20-1.90 (m, 5H, —OOC—CH₂CH(CH₃)CH₂—), 0.72 (d, 3H, —OOC—CH₂CH(CH₃)CH₂—).

### Example 57

### Synthesis of Medhesive-127 (PEG20k-(MGA(Ac)₂DMe)₈)

5.05 g (0.238 mmol) of PEG20k-(MGAe)₈ was dissolved in 20 mL of chloroform. 0.834 g (3.04 mmol) of 3,4-Diacetoxyphenethylamine hydrochloride, 0.4069 g (3.02 mmol) of HOBt and 1.1427 g (3.01 mmol) of HBTU was dissolved in 14 mL DMF. The two solution were added together. An additional 14 mL of DMF was added to the reaction. When the solution was clear, 0.63 mL (4.52 mmol) of triethylamine was added and the reaction was allowed to stir for ˜1 hour. The reaction was gravity filtered into 300 mL of diethyl ether and placed at ˜4° C. for ˜18 hours. The precipitate was suction filtered and dried under vacuum for ˜3 days. The material was dissolved in ˜50 mL of 12.1 mM HCl and placed in 3500 MWCO dialysis tubing. This was dialyzed against 3.5 L of nanopure water acidified with 0.350 mL of concentrated HCl. The dialysate was changed 11 times over 48 hours. The dialysate was changed to nanopure water and changed once over 2 hours. The polymer solution was gravity filtered, frozen and placed on a lyophilizer until dry. 3.30 g of material was obtained (LN005990). ¹H NMR (400 MHz, D₂O): δ 7.15-7.0 (m, 3H, C₆H₃—), 4.13 (t, 2H, —O—CH—CH₂—PEG-), 3.8-3.0 (m, 228H, PEG, —CH₂—CH₂—C₆H₃), 2.73 (m, 2H, NHCO—CH₂—CH—), 2.21 (s, 6H, C₆H₃(OOCH₃)₂), 2.10-1.90 (m, 5H, —OOC—CH₂CH(CH₃)CH₂—), 0.73 (d, 3H, —OOC—CH₂CH(CH₃)CH₂—).

### Example 58

### Synthesis of Medhesive-128 (PEG20k-(MGAPEAe)₈)

5.01 g (0.238 mmol) of PEG20k-(MGAe)₈ was dissolved in 20 mL of chloroform. 0.484 g (3.07 mmol) of phenethylamine hydrochloride, 0.407 g (3.01 mmol) of HOBt and 1.1488 g (3.03 mmol) of HBTU was dissolved in 14 mL DMF. The two solution were added together. An additional 13 mL of DMF was added to the reaction. When the solution was clear, 0.63 mL (4.52 mmol) of triethylamine was added and the reaction was allowed to stir for ˜1 hour. The reaction was gravity filtered into 300 mL of diethyl ether and placed at ˜4° C. for ˜18 hours. The precipitate was suction filtered and dried under vacuum for ˜3 days. The material was dissolved in ˜50 mL of 12.1 mM HCl and placed in 3500 MWCO dialysis tubing. This was dialyzed against 3.5 L of nanopure water acidified with 0.350 mL of concentrated HCl. The dialysate was changed 11 times over 48 hours. The dialysate was changed to nanopure water and changed once over 2 hours. The polymer solution was gravity filtered, frozen and placed on a lyophilizer until dry. 2.90 g of material was obtained (LN007001). ¹H NMR (400 MHz, D₂O): δ 7.3-7.0 (m, 5H, C₆H₅—), 4.14 (t, 2H, —O—CH—CH₂—PEG-), 3.8-3.0 (m, 228H, PEG, —CH₂—CH₂—C₆H₅), 2.71 (m, 2H, NHCO—CH₂—CH₂—), 2.10-1.90 (m, 5H, —OOC—CH₂CH(CH₃)CH₂—), 0.73 (d, 3H, —OOC—CH₂CH(CH₃)CH₂—).

### Example 59

### Synthesis of Medhesive-129 (PEG20k-(LysDMHA2)₈)

9.98 g (0.475 mmol) of PEG20k-(Lyse)₈ was dissolved in 65 mL of chloroform and 35 mL of DMF. 3.1127 g (14.81 mmol) of 3,4-dimethoxyhydrocinnamic acid. 2.007 g (14.84 mmol) of HOBt and 5.611 g (14.80 mmol) of HBTU was added to the reaction and stirred until completely dissolved. When the solution was clear, 2.07 mL (14.85 mmol) of triethylamine was added and the reaction was allowed to stir for ˜90 minutes. The reaction was gravity filtered into 600 mL of diethyl ether and placed at ˜4° C. for ˜15 hours. The precipitate was suction filtered and dried under vacuum for ˜4 days. The material was dissolved in ˜100 mL of 12.1 mM HCl, gravity filtered and placed in 3500 MWCO dialysis tubing. This was dialyzed against 3.5 L of nanopure water acidified with 0.400 mL of concentrated HCl. The dialysate was changed 5 times over 24 hours. The dialysate was changed to nanopure water and changed 5 times over 24 hour. The polymer solution was gravity filtered, frozen and placed on a lyophilizer until dry. 6.50 g of material was obtained (LN006530). ¹H NMR (400 MHz, D2O/TMS): δ 6.8-6.5 (m, 6H, —C₆H₃—), 4.15 (t, 2H, —O—CH—CH₂—PEG-), 3.8-3.25 (m, 238H, PEG, —C₆H₃—O—CH₃), 3.0-0.5 (m, 16H, —OCOCH(NHCH₂CH₂—)CH₂CH₂CH₂CH₂—NH—CH₂CH₂—).

### Example 60

### Synthesis of Medhesive-130 (PEG20k-(LysHCA2)₈)

10 g (0.475 mmol) of PEG20k-(Lyse)₈ was dissolved in 65 mL of chloroform and 35 mL of DMF. 2.221 g (14.8 mmol) of hydrocinnamic acid, 1.995 g (14.8 mmol) of HOBt and 5.6173 g (14.8 mmol) of HBTU was added to the reaction and stirred until completely dissolved. When the solution was clear, 2.07 mL (14.85 mmol) of triethylamine was added and the reaction was allowed to stir for ˜90 minutes. The reaction was gravity filtered into 600 mL of diethyl ether and placed at ˜4° C. for ˜8 hours. The precipitate was suction filtered and dried under vacuum for ˜3 days. The material was dissolved in ˜100 mL of 12.1 mM HCl, gravity filtered and placed in 3500 MWCO dialysis tubing. This was dialyzed against 3.5 L of nanopure water acidified with 0.400 mL of concentrated HCl. The dialysate was changed 5 times over 24 hours.

The dialysate was changed to nanopure water and changed 5 times over 24 hours. The polymer solution was gravity filtered, frozen and placed on a lyophilizer until dry. 7.30 g of material was obtained (LN006567). ¹H NMR (400 MHz, D2O/TMS): δ 7.25-7.1 (m, 6H, —C₆H₅—), 4.15 (t, 2H, —O—CH—CH₂—PEG-), 3.8-3.25 (m, 238H, PEG, —C₆H₅—O—CH₃), 3.0-0.5 (m, 16H, —OCOCH(NHCH₂CH₂)CH₂CH₂CH₂CH₂—NH—CH₂CH₂—).

### Example 61

### Synthesis of Medhesive-134 (PEG20k-(3M-4NBA)₈)

1.895 g (9.6 mmol) of 3-methoxy-4-nitrobenzoic acid and 3.6382 g (9.6 mmol) of HBTU were dissolved in 10 mL of chloroform and 40 mL of DMF. 1.338 mL (9.6 mmol) of triethylamine was added and the reaction was allowed to stir for 15 minutes. 14.994 g (0.75 mmol) of PEG20k-(NH₂)₈ was dissolved in 40 mL chloroform and 60 mL of DMF followed by the addition of 0.836 mL (6 mmol) of triethylamine. The PEG/TEA solution was transferred to an addition funnel and added dropwise over 30 minutes to the HBTU reaction. The reaction was allowed to stir for an additional 90 minutes. The reaction was gravity filtered into 1.5 L of diethyl ether and placed at 4° C. for 20 hours. The precipitate was suction filtered and placed under vacuum for 5 hours. The polymer was dissolved in 170 mL of nanopure water. The solution was gravity filtered and placed in 3500 MWCO dialysis tubing. The solution was dialyzed against 3.5 L of nanopure water. The dialysate was changed 7 times over the next 24 hours. The polymer was gravity filtered, frozen, and placed on a lyophilizer until dry. ˜12 g of material was obtained (LN007265). ¹H NMR conformed to structure.

### Example 62

### Synthesis of Medhesive-135 (PEG20k-(3H-4NBA)₈)

1.7612 g (9.6 mmol) of 3-hydroxy-4-nitrobenzoic acid and 3.6377 g (9.6 mmol) of HBTU were dissolved in 10 mL of chloroform and 40 mL of DMF. 1.338 mL (9.6 mmol) of triethylamine was added and the reaction was allowed to stir for 15 minutes. 15.01 g (0.75 mmol) of PEG20k-(NH₂)₈ was dissolved in 40 mL chloroform and 60 mL of DMF followed by the addition of 0.836 mL (6 mmol) of triethylamine. The PEG/TEA solution was transferred to an addition funnel and added dropwise over 30 minutes to the HBTU reaction. The reaction was allowed to stir for an additional 90 minutes. The reaction was gravity filtered into 1.5 L of diethyl ether and placed at 4° C. for 20 hours. The precipitate was suction filtered and placed under vacuum for 6 hours. The polymer was dissolved in 177 mL of nanopure water. The solution was gravity filtered and placed in 3500 MWCO dialysis tubing. The solution was dialyzed against 3.5 L of nanopure water. The dialysate was changed 7 times over the next 24 hours. The polymer was gravity filtered, frozen, and placed on a lyophilizer until dry. 12.5 g of material was obtained (LN007278). ¹H NMR conformed to structure.

### Example 63

### Synthesis of Medhesive-149 (PEG10k-(ADA-DOHA)₄)

24.99 g (2.283 mmol) of PEG10k-(ADA)₄, 2.006 g (10.96 mmol) of 3,4-dihydroxyhydrocinnamic acid and 4.173 g (10.96 mmol) of HBTU was dissolved in 125 mL of DMSO while stirring at 52° C. 2.80 mL (20.09 mmol) of triethylamine was added and the reaction was allowed to stir for ˜90 minutes. The solution was added to 250 mL of methanol and placed in 2000 MWCO dialysis tubing. This was dialyzed against 2.5 L of nanopure water acidified with 0.25 mL of concentrate HCl. The dialysate was changed 10 times over 40 hours. The dialysate was changed to nanopure water and changed 4 times over the next 4 hours. The polymer solution was frozen and placed on a lyophilizer until dry. 24.87 g of material was obtained (LN012117). ¹H NMR (400 MHz, DMSO/TMS): δ 8.68 (s, 1H, —C₆H₃(OH)₂), 8.58 (s, 1H, —C₆H₃(OH)₂), 7.71 (s, 1H, —OOC(CH₂)₁₀—NH—CO—), 6.6 (d, 1H, —C₆H₃(OH)₂), 6.54 (s, 1H, —C₆H₃(OH)₂), 6.4 (d, 1H, —C₆H₃(OH)₂), 4.11 (t, 2H, —CH₂—OOC(CH₂)₁₀—), 3.8-3.2 (m, 226H, PEG), 2.99 (t, 2H, —OOCCH₂(CH₂)₉—NH₂), 2.59 (m, 2H, —NHOC—CH₂—CH₂—), 2.25 (m, 4H, —OOC(CH₂)₉—CH₂—NH₂, NHOC—CH—CH₂—), 1.51 (m, 2H, —OOCCH₂CH₂(CH₂)₈—NH₂), 1.33 (m, 2H, —OOC (CH₂)₈—CH₂CH₂—NH₂), 1.21 (m, 12H, —OOCCH₂CH₂(CH₂)₆CH₂CH₂—NH₂).

### Example 64

### Synthesis of Medhesive-155 (PEG20k-(GABA-DABA)₈)

40.00 g (1.91 mmol) of PEG20k-(GABA)₈, 8.602 g (24.46 mmol) of di-Boc-3,4-diaminobenzoic acid and 9.27 g (24.46 mmol) of HBTU was dissolved in 240 mL DMF and 120 mL of chloroform while stirring. 5.54 mL (39.74 mmol) of triethylamine was added and the reaction was allowed to stir for ˜3 hours. The reaction was gravity filtered into 3.2 L of MTBE and placed at ˜4° C. for ˜22 hours. The precipitate was suction filtered and dried under vacuum for 25 hours (LN012135). This intermediate was called PEG20k-(GABA-Boc-DABA)₈. 45 g of PEG20k-(GABA-Boc-DABA)₈ was dissolved in 180 mL of chloroform under argon. 200 mL of 4M HCl in Dioxane was added to the solution and allowed to stir for 30 minutes under argon. The solvent was roto evaporated off. The resulting polymer was dissolved in 800 mL of nanopure water and placed in 2000 MWCO dialysis tubing. This was dialyzed against 6 L of nanopure water. The dialysate was changed 6 times over 22 hours. The polymer solution was suction filtered, frozen and placed on a lyophilizer until dry. 36.98 g of material was obtained (LN012143). ¹H NMR (400 MHz, DMSO/TMS): δ 8.02 (s, 1H, —NHOCC₆H₃(NH₂)₂), 7.25 (s, 1H, —C₆H₃(NH₂)₂), 7.17 (d, 1H, —C₆H₃(NH₂)₂), 7.0-5.0 (d, 5H, —C₆H₃(NH₂)₂), 4.09 (t, 2H, —CH₂—OOC—CH₂—), 3.8-3.2 (m, 228H, PEG, —OOCCH₂CH₂CH₂—NH—), 2.33 (m, 2H, —OOCCH₂CH₂CH₂—NH—), 1.72 (m, 2H, —OOCCH₂CH₂CH₂—NH—).

### Example 65

### Synthesis of Medhesive-160 (PEG20k-(β-Ala-DABA)₈)

40 g (1.91 mmol) of PEG20k-(β-Ala)₈, 8.67 g (24.46 mmol) of di-Boc-3,4-diaminobenzoic acid and 9.25 g (24.46 mmol) of HBTU was dissolved in 240 mL DMF and 120 mL of chloroform while stirring. 5.54 mL (39.74 mmol) of triethylamine was added and the reaction was allowed to stir for ˜2 hours. The reaction was gravity filtered into 3.0 L of MTBE and placed at ˜4° C. for ˜23 hours. The precipitate was suction filtered and dried under vacuum for ˜17 hours (LN012428). This intermediate was called PEG20k-(β-Ala-Boc-DABA)₈. 51.4 g of PEG20k-(β-Ala-Boc-DABA)₈ was dissolved in 230 mL of chloroform under argon. 260 mL of 4M HCl in Dioxane was added to the solution and allowed to stir for 30 minutes under argon. The solvent was roto-evaporated off. The resulting polymer was dissolved in 1 L of nanopure water and placed in 2000 MWCO dialysis tubing. This was dialyzed against 9 L of nanopure water. The dialysate was changed 6 times over 24 hours. The polymer solution was suction filtered, frozen and placed on a lyophilizer until dry. 36.68 g of material was obtained (LN012434). ¹H NMR (400 MHz, DMSO/TMS): δ 8.02 (s, 1H, —NHOCC₆H₃(NH₂)₂), 7.25 (s, 1H, —C₆H₃(NH₂)₂), 7.17 (d, 1H, —C₆H₃(NH₂)₂), 7.0-5.0 (d, 5H, —C₆H₃(NH₂)₂), 4.12 (t, 2H, —CH₂—OOC—CH₂—), 3.8-3.2 (m, 228H, PEG, —OOCCH₂CH₂—NH—), 2.55 (m, 2H, —OOCCH₂CH₂—NH—).

### Example 66

### Synthesis of Medhesive-161 (PEG20k-(PI-Ala-DOHA)₈)

40.05 g (1.91 mmol) of PEG20k-(β-Ala)₈, 3.357 g (18.34 mmol) of 3,4-dihydroxyhydrocinnamic acid and 6.95 g (18.34 mmol) of HBTU was dissolved in 240 mL DMF and 120 mL of chloroform while stirring. 4.69 mL (33.62 mmol) of triethylamine was added and the reaction was allowed to stir for ˜90 minutes. The reaction was gravity filtered into 3.0 L of MTBE and placed at ˜4° C. for ˜20 hours. The precipitate was suction filtered and dried under vacuum for ˜21 hours. The resulting polymer was dissolved in 400 mL of nanopure water and placed in 2000 MWCO dialysis tubing. This was dialyzed against 10 L of nanopure water acidified with 1 mL of concentrate HCl. The dialysate was changed 7 times over 23 hours. The dialysate was changed to nanopure water and changed 4 times over the next 5 hours. The polymer solution was frozen and placed on a lyophilizer until dry. 39.50 g of material was obtained (LN012430). ¹H NMR (400 MHz, D2O/TMS): δ 6.68 (d, 1H, —C₆H₃(OH)₂), 6.60 (s, 1H, —C₆H₃(OH)₂), 6.51 (d, 1H, —C₆H₃(OH)₂), 4.09 (t, 2H, —CH₂—OOC—CH₂—), 3.8-3.2 (m, 228H, PEG, —OOCCH₂CH₂—NH—), 2.65 (t, 2H, —OOCCH₂CH₂—NHOC—CH₂CH₂—) 2.34 (m, 4H, —OOCCH₂CH₂—NHOC—CH₂CH₂—).

### Example 67

### GPC Analysis of MPEG5k-(PD)

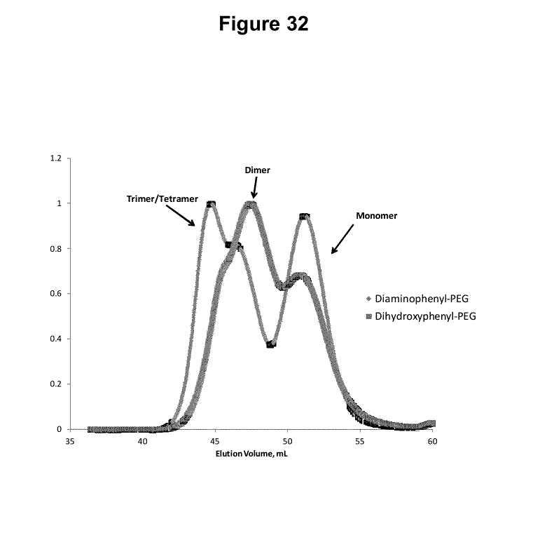

Gel permeation chromatography (GPC) is used for analysis of linear polymers synthesized with different PD endgroups to provide information about molecular, weight, size distribution, the number of times an adhesive endgroup reacts with itself, and crosslink functionality under oxidative conditions. (Initial steps of ferulic acid polymerization by lignin peroxidase. Journal of Biological Chemistry. 276: 2001:18734-18741). For example, FIG. 32 shows concentration chromatograms for a dihydroxyphenyl functionalized linear methoxy terminated PEG (Surphys-074) and a diamino functionalized linear methoxy terminated PEG (Surphys-066). FIG. 32 illustrates that at a fixed IO₄⁻:endgroup ratio, formation of trimers and tetramers predominates with the diamino functionality whereas dimers are the principal fraction with the typical dihydroxy endgroup, and indicates that coatings containing diamino endgroups may be more mechanically robust due to enhanced intermolecular interactions and polymer surface interaction.

### Example 68

### Adhesive Polymer Gelation Time Determination

A known amount of polymer was dissolved in 2× phosphate buffered saline at a desired concentration. A solution of sodium periodate was prepared at a given concentration of IO₄⁻:PD. 100 μL of polymer solution was pipetted into a test tube and stirred with a micro stir bar at 300 rpm. As 100 μL of the sodium periodate cross-linking solution was pipetted into the polymer solution, a timer is started. When the micro stir bar stopped spinning, the timer was stopped, and the time was recorded. The gelation times from three samples are used to calculate a mean and standard deviation. The values of these experiments are shown in Table 1. All values were collected at 15 Wt % polymer.

TABLE 1
PEG20k-(PD)₈ Derivatives: Characterization and Physical Properties

| | | | | [symbol] | | [symbol] | [symbol] | | | |
|---|---|---|---|---|---|---|---|---|---|---|
| [symbol] | Compound | | | polymer | | [symbol] | [symbol] | [symbol] | | |
| | Name | Linker | PEG | [symbol] | pH | Polymer | Polymer | [symbol] | | |
| 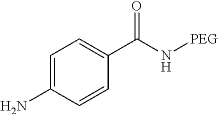 | Surphys- 059 | N/A | PEG20k- (NH₂) [symbol] | 0.310 (¹H NMR) | 6.21 | Did Not Gel | Not Acquired (Hydrogel Does not Form) | Not Acquired (Hydrogel Does not Form) | | |
| 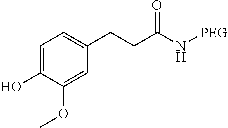 | Surphys- 061 | N/A | PEG20k- (NH₂)₈ | 0.338 (¹H NMR) | 7.41 | 46.1+/ −1.3 [0.5] 23.0+/ −0.9 [1.0] 12.0+/ −0.3 [2.0] | 81.6+/ −23.9 [0.5] 176.1+/ −34.0 [1.0] 147.4+/ −56.0 [2.0] | Not Acquired | | |
| 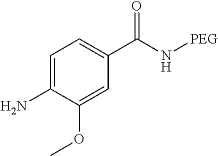 | Surphys- 062 | N/A | PEG20k- (NH₂)₈ | 0.305 (¹H NMR) | 6.15 | 76.5+/ −3.5 [3.0] 42.6+/ −2.8 [4.0] | 181.3+/ −51.0 [3.0] 157.7+/ −42.8 [4.0] | Not Acquired | | |
| 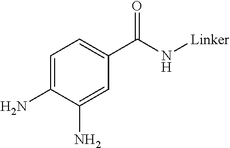 | Surphys- 065 | N/A | PEG20k- (NH₂)₈ | 0.295 (¹H NMR) | 5.05 | 29.1+/ −1.3 [0.25] 6.5+/ −0.3 [0.5] N/A | 8.0+/ −7.8 [0.25] 83.3+/ −29.7 [0.5] 41.1+/ −19.0 [1.0] | Not Acquired | | |
| 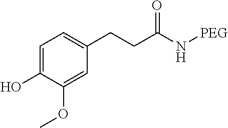 | Surphys- 068 | N/A | PEG20k- (NH₂)₈ | 0.285 (¹H NMR) | 7.43 | 28.3+/ −0.9 [0.25] 16.9+/ −0.4 [0.5] 11.2+/ −0.3 [1.0] | 4.4+/ −6.5 [0.25] 60.5+/ −44.7 [0.5] 96.6+/ −61.5 [1.0] | Not Acquired | | |
| 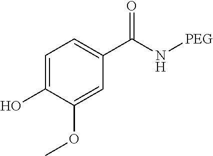 | Surphys- 069 | N/A | PEG20k- (NH₂)₈ | 0.297 (¹H NMR) | 7.28 | Did Not Gel | Not Acquired (Hydrogel Does not Form) | Not Acquired (Hydrogel Does not Form) | | |
| 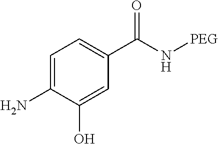 | Surphys- 077 | N/A | PEG20k- (NH₂)₈ | 0.301 (¹H NMR) | 5.43 | 0 sec [0.25] | 5.0+/ −8.0 [0.5] | Not Acquired | | |
| 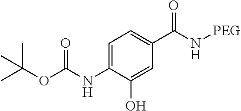 | Suryphys- 079 | N/A | PEG20k- (NH₂)₈ | 0.338 (¹H NMR) | 7.09 | 53.2+/ −1.9 [0.5] 33.5+/ −1.5 [1.0] | 36.5+/ −14.5 [0.5] 33.3+/ −8.1 [1.0] | Not Acquired | | |
| | Compound | | | | | | | | | |
| [symbol] | Name | Linker | PEG | [symbol] | pH | [symbol] | [symbol] | [symbol] | | |
| 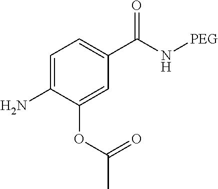 | Surphys- 081 | N/A | PEG20k- (NH₂)₈ | 0.295 (¹H NMR) | 7.11 | 49.9 s [0.5] 32.3+/ −0.6 s [1.0] | 47.0+/ −20.8 [0.5] 37.3+/ −22.5 [1.0] | Not Acquired | | |
| 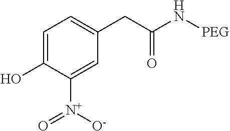 | Surphys- 083 | N/A | PEG20k- (NH₂)₈ | 0.293 (¹H NMR) | 6.37 | Did Not Gel | Not Acquired (Hydrogel Does not Form) | Not Acquired (Hydrogel Does not Form) | | |
| 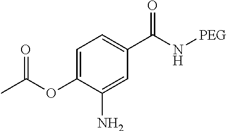 | Surphys- 085 | N/A | PEG20k- (NH₂)₈ | 0.245 (¹H NMR) | 6.64 | 113.9+/ −2.8 [1.0] | 54.9+/ −13.9 [1.0] | Not Acquired | | |
| 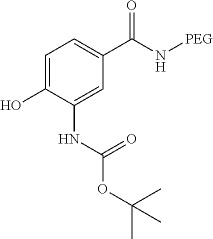 | Surphys- 087 | N/A | PEG20k- (NH₂)₈ | 0.258 (¹H NMR) | 6.99 | 75.9+/ −2.9 [1.0] | 141.9+/ −34.5 [1.0] | Not Acquired | | |
| 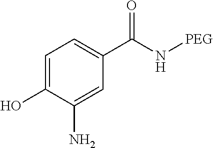 | Surphys- 089 | N/A | PEG20k- (NH₂)₈ | 0.265 (¹H NMR) | 4.60 | 0.5+/ −0.3 [0.5] | 19.4+/ −10.0 [0.5] | Not Acquired | | |
| 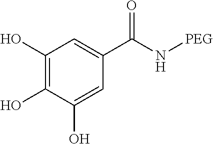 | Med- hesive- 077 | N/A | PEG20k- (NH₂)₈ | 0.263 (¹H NMR) | 7- 7.4 | Imme- diate | Not Acquired- Clogged tip when spraying | Not Acquired | | |
| 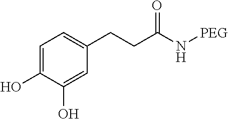 | Med- hesive- 079 | N/A | PEG20k- (NH₂)₈ | 0.316 (¹H NMR) | 7.38 | 1.3+/ −0.1 [0.25] 0.9+/ −0 [0.5] N/A | 13.3+/ −7.6 [0.25] 16.1+/ −8.0 [0.5] 21.5+/−5.7 [0.5] 40 mM HCl added | Not Acquired | | |
| 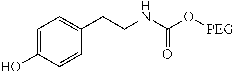 | Med- hesive- 117 | N/A | PEG20k- (OH)₈ | 0.360 (Theo- retical Value Used) | N/A | Not Acquired | Not Acquired | Not Acquired | | |
| | Compound | | | | | | | | | |
| [symbol] | Name | Linker | PEG | [symbol] | pH | [symbol] | [symbol] | [symbol] | | |
| 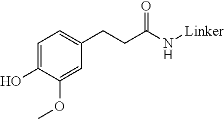 | Med- hesive- 120 | Lysine | PEG20k- (OH)₈ | 0.531 (UV- VIS @ 2.80 nm) | 7.44 | 19.3+/ −0.8 [0.5] Not Acquired | 155.3+/ −10.4 [0.5] 127.4+/ −28.9 [1.0] | Not Acquired | | |
| 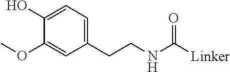 | Med- hesive- 121 | Methyl Glutaric Acid | PEG20k- (OH)₈ | 0.323 (UV- VIS @ 280 nm) | 7.48 | 83.8+/ −0.9 [0.5] 72.0+/ −0 [0.75] 60.0+/ −0 [1.0] | 116.8+/ −14.6 [0.5] 156.5+/ −45.0 [0.75] 215.4+/ −33.9 [1.0] | Not Acquired | | |
| 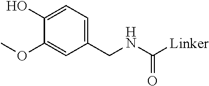 | Med- hesive- 122 | Methyl Glutaric Acid | PEG20k- (OH)₈ | 0.342 (UV- Vis @ 280 nm) | N/A | Not Acquired | Not Acquired | Not Acquired | | |
| 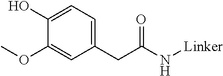 | Med- hesive- 123 | Lysine | PEG20k- (OH)₈ | 0.595 (UV- VIS @ 280 nm) | N/A | Not Acquired | Not Acquired | Not Acquired | | |
| 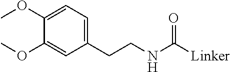 | Med- hesive- 125 | Methyl Glutaric Acid | PEG20k- (OH)₈ | 0.319 (UV- VIS @ 280 nm) | N/A | Did not gel | Not Acquired (Hydrogel Does not Form) | Not Acquired (Hydrogel Does not Form) | | |
| 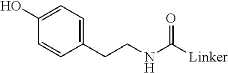 | Med- hesive- 126 | Methyl Glutaric Acid | PEG20k- (OH)₈ | 0.118 (UV- VIS @ 280 nm) | N/A | Not Acquired | Not Acquired | Not Acquired | | |
| 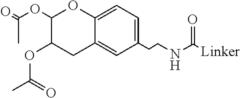 | Med- hesive- 127 | Methyl Glutaric Acid | PEG20k- (OH)₈ | 0.360 (Theo- retical Value Used) | N/A | Not Acquired | Not Acquired | Not Acquired | | |
| 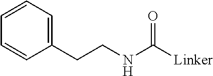 | Med- hesive- 128 | Methyl Glutaric Acid | PEG20k- (OH)₈ | 0.360 (Theo- retical Value Used) | N/A | Not Acquired | Not Acquired | Not Acquired | | |
| | Compound | | | | | | | | [symbol] | |
| [symbol] | Name | Linker | PEG | [symbol] | pH | [symbol] | [symbol] | [symbol] | 37° C. | 55° C. |
| 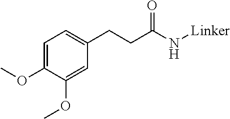 | Med- hesive- 129 | Lysine | PEG20k- (OH)₈ | 0.571 (UV- (VIS @ 280 nm) | N/A | No Acquired | Not Acquired | Not Ac- quired | Not Ac- quired | Not Ac- quired |
| 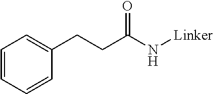 | Med- hesive- 130 | Methyl Glutaric Acid | PEG20k- (OH)₈ | N/A | N/A | Not Acquired | Not Acquired | Not Ac- quired | Not Ac- quired | Not Ac- quired |
| 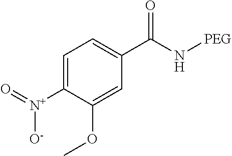 | Med- hesive- 134 | N/A | PEG20k- (NH₂)₈ | 0.355 (UV- VIS@ 300 nm) | N/A | Not Acquired | Not Acquired | Not Ac- quired | Not Ac- quired | Not Ac- quired |
| 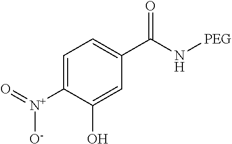 | Med- hesive- 135 | N/A | PEG20k- (NH₂)₈ | N/A | N/A | Not Acquired | Not Acquired | Not Ac- quired | Not Ac- quired | Not Ac- quired |
| 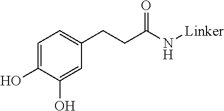 | Med- hesive 149 | 11- Amino- un- dec- anoic Acid | PEG10k- (OH)₄ | 0.333 (Theo- retical value used) | 7- 7.4 | 20.7+/ −3.0 [0.5] | 75.5+/ −28.5 [0.5] | Not Ac- quired | Not Ac- quired | Not Ac- quired |
| 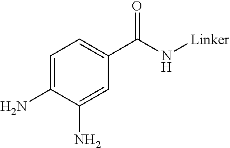 | Med- hesive- 155 | ν- amino- butyric acid | PEG20k- (OH)₈ | 0.364 (Theo- retical value used) | ~4.5 | 2.7+/ −0.1 [0.5] | 99.6+/ −22.4 [0.5] | 19.7+/ −3.6% | Not Ac- quired | 11 days |
| 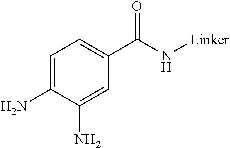 | Med- hesive- 160 | B- Alanine | PEG20k- (OH)₈ | 0.366 (Theo- retical value used) | 4.88 | 2.4+/ −0.1 [0.5] | 42.1+/ −19.2 [0.5] | 44.5+/ −7.7% | 44 days | 5-6 days |
| 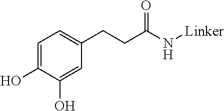 | Med- hesive- 161 | B- Alanine | PEG20k- (OH)₈ | 0.362 (Theo- retical value used) | 7.13 | 9.2+/ −0.7 | 102.3+/ −31.8 [0.5] | 39.1+/ −4.2% | 58-60 days | 6 days |

[symbol]

indicates data missing or illegible when filed

### Example 69

### Adhesive Polymer pH Determination

The pH of the polymer solution was measured by weighing out 750 mg of compound into a glass vial. The compound was dissolved completely into 2.5 mL of 2×PBS buffer. The pH was measured with a pH meter which had been calibrated. The pH of the

### Example 70

### Adhesive Polymer Percent Swelling Determination

A known amount of polymer was dissolved in 2× phosphate buffered saline at the desired concentration and loaded into a 3 mL syringe. An additional 3 mL syringe was filled with a solution of sodium periodate prepared at a concentration of 0.5 IO₄⁻: DHP. Both the polymer solution syringe and the sodium periodate syringe, in a volumetric ratio of 1:1 were connected to a y-adaptor and secured with a syringe holder and plunger lock. A spray tip was connected and a mixture of the two solutions is expressed onto the surface of a PTFE sheet. The hydrogels produced were allowed to cure for approximately 10 minutes, then are cut into 6 approximately equal pieces and placed into 6 glass vials. The relaxed weight of each polymer gel was collected (Wᵣ). 10 mL of phosphate buffered saline was then added to each glass vial and the gels were allowed to swell at 37 degrees Celsius for 24 hours. After which, the phosphate buffered saline was decanted from the vials and the interior of the vial was dried. The swollen weight of the gel was collected (Wₛ). The swollen gels were then placed in a vacuum desiccator for 48 hours and weighed again (W_{d}). The percent volumetric swelling ratio (Vᵣ) was then calculated as follows:

$$R = \frac{{V}_{s}}{{V}_{r}}$$

$${V}_{s} = \frac{{W}_{d}}{{ρ}_{PEG}} + \frac{{W}_{s} - {W}_{d}}{{ρ}_{Solvent}}$$

$${V}_{r} = \frac{{W}_{d}}{{ρ}_{PEG}} + \frac{{W}_{r} - {W}_{d}}{{ρ}_{Solvent}}$$

where ρ_{PEG} is the density of the polymer (1.123 g/mL) and ρ_{Solvent} is the density of the solvent (1.123 g/mL for water). Swelling values are shown in Table 1. All values collected were at 15 Wt % polymer.

### Example 71

### Adhesive Burst Strength Determination

Fresh crosslinked, collagen substrate (F_{TYPE} Sausage Casing, Nippi Inc.) was prepared by hydrating and washing in a mild detergent for 20 min. 40 mm circles were cut and a 2-mm circular defect was cut in the center of each circle. The samples were stored in phosphate buffered saline until use. A known amount of polymer was dissolved in 2× phosphate buffered saline at the desired concentration and loaded into a 3 mL syringe. An additional 3 mL syringe was filled with a solution of sodium periodate prepared at a concentration of 0.5 IO₄⁻: PD. Both the polymer solution syringe and the sodium periodate syringe, in a volumetric ratio of 1:1 were connected to a y-adaptor and secured with a syringe holder and plunger lock. The collagen substrates were placed on a petroleum coated PTFE sheet, and covered with a 3.5 cm diameter PTFE mask with a 1.5 cm hole. A spray tip was connected and a mixture of the two solutions was expressed into the PTFE mask hole. The sample was then covered with a petroleum coated glass slide, and a 100 gram weight was placed on top to ensure uniform thickness. The samples were allowed to cure approximately 10 minutes before they were placed in phosphate buffered saline at 37 degrees Celsius and incubated for one hour. The samples were then burst tested in accordance with ASTM F2392 entitled, “Standard Test Method for Burst Strength of Surgical Sealants”. The pressure required to burst through the hydrogel was then recorded. Burst strength pressure values are shown in Table 1. All values collected were at 15 Wt % polymer.

### Example 72

### Sprayability of Adhesive Hydrogels

Solutions of Medhesive were prepared at 15 Wt % in 2×PBS buffer at a 0.5 IO₄⁻:PD ratio. For spray testing it is optimal to have gelation times under 3 seconds. At the same time, gelation can not be so quick that it clogs the tip in the spray device. It was found that a gelation time of ˜2.5-3 seconds produces optimal results on spray testing. To obtain the proper gelation time the pH of the formulation may be increased (faster gelation) or decreased (slower gelation). The gelation time of optimal formulations are shown in Table 2.

TABLE 2
Formulation Optimization for Sprayability Testing

| Polymer | Diluent | Gelation Time (sec) |
|---|---|---|
| M102 | 2xPBS + 10 mM NaOH | 2.7 +/− 0.29 |
| M069 | 2xPBS + 15 mM NaOH | 2.8 +/− 0.30 |
| M155 | 2xPBS | 2.7 +/− 0.10 |
| M160 | 2xPBS + 5 mM HCl | 2.8 +/− 0.12 |
| M161 | 2xPBS + 10 mM NaOH | 2.9 +/− 0.38 |

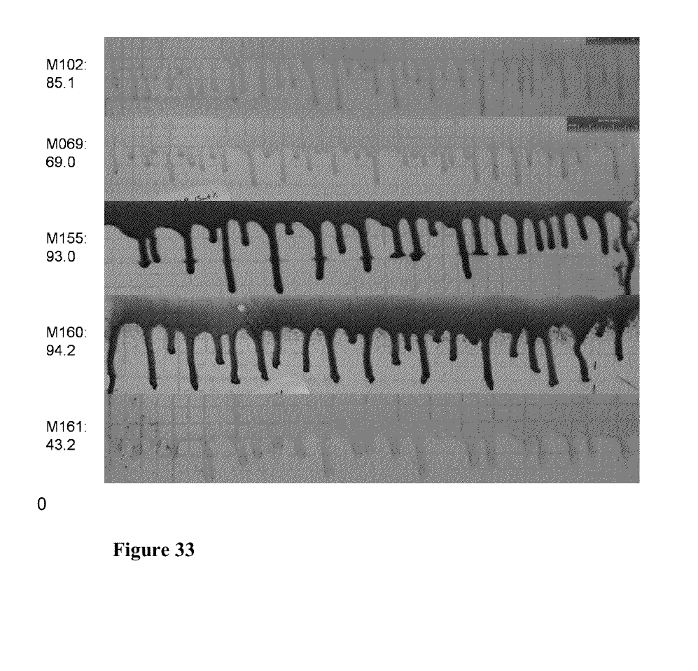

Formulations cited in Table 2 were sprayed onto a 90° surface at a velocity of 65 mm/s and an acceleration rate of 10,000 mm/s2. The sweep length was 500 mm and the flow rate was 40 mL/min. The drips in a 30 cm section were measured and the drip quotient was measured using the following formula: Sqrt(#drips)*(average drip length)² FIG. 33 shows the results of these experiments.

### Example 73

### Sterilization of Medhesive Properties

Medhesive kits consisting of the spray device, Medhesive, 2×PBS, NaIO₄, and nanopure water were underwent E-Beam sterilization (25kGy). Their physical properties were measured and the results are shown in Table 3.

TABLE 3
Effect of Sterilization on Medhesive Formulations

| | | | | | | Pilot | | |
|---|---|---|---|---|---|---|---|---|
| | | | Gelation | | Volumetric | Degradation | | |
| | Polymer | pH | (sec ) | Burst (mmHg) | Swelling | (55° C.) | (37° C.) | Drip Quotient |
| Pre- | M160 | 4.88 | 2.8 +/− 0.12 | 88.1 +/− 22.8 | 47% +/− 3% | 5 d | 49 d | 122.6 |
| Sterilization | M161 | 7.13 | 2.9 +/− 0.38 | 94.1 +/− 23.6 | 59% +/− 4% | 6 d | 59 d | 45.0 |
| Post- | M160 | 4.85 | 2.7 +/− 0.19 | 77.5 +/− 39.0 | 40% +/− 2% | 4 d | 42 d | 56.6 |
| Sterilization | M161 | 7.98 | 2.5 +/− 0.28 | 93.5 +/− 29.2 | 56% +/− 4% | 6 d | 67 d | 50.4 |

Minimal to no effect of sterilization was observed on gelation, burst testing, swelling and degradation. A large difference was noticed for the drip quotient with Medhesive-160, however, this effect was positive in nature.

### Example 74

### Degradation time of Adhesive Hydrogels

To assess the degradation time of adhesive hydrogels, polymer was weighed into a syringe and linked to another syringe containing the appropriate amount of buffer. The two syringes were mixed via a blending connector until the entire polymer was dissolved. A solution of NalO₄ was prepared and loaded into a syringe. The mixed polymer syringe and the NaIO₄ syringe were connected to a Y-adapter and a spray tip, syringe holder, and plunger lock were attached. The Medhesive polymer was then expressed onto a PTFE sheet and allowed to cure on the bench top for approximately 10 minutes. The hydrogels were then cut into pieces approximately 1 cm×1 cm. Each piece was then placed into a glass vial of known weight and the relaxed weight was collected. The polymer was then covered with 10 mL PBS and placed in an incubator, at a temperature of 37° C. or 55° C. Periodically the vials were removed, the water emptied, and then remaining gel weighed. The remaining gel was then dried under vacuum for 48 hours and weighed again. The change in mass was calculated. Results for degradation rate can be seen in Table 1. and Table 3.

### Example 75

### Degradation Rate and Polymer Structure

As shown in Tables 1. and 3., Medhesive-155, which contains a γ-aminobutyric acid linker, degrades at a slower rate than Medhesive-160, which contains a f-alanine linker. The difference in degradation is due, for example, to the number of alkane units (—CH₂—). Where Medhesive-155 has 3-CH₂— units, Medhesive-160 only has 2. This results in Medhesive-161 degrading faster than Medhesive-155. Accordingly, Medhesive-149, which contains 10-CH₂-units, would degrade very slowly. Moreover, the degradation rate differs between Medhesive-160 and Medhesive-161 due in part to the different PD's used between Medhesive-160 (3,4-diaminobenzoic acid) and Medhesive-161 (3,4-dihydroxhydrocinnamic acid).

### Example 76

### Synthesis of Medhesive-233 (PEG20k-(GABA-DOHA)₈)

200 g of PEG_{20K}(GABA)₈ was added to a 3 L round-bottom flask and dissolved in 600 mL of chloroform and 600 mL of DMF. In a separate flask 16.4 g of DOHA was dissolved in 500 mL of N,N-dimethylformamide (DMF) and slowly added to the flask containing PEG_{20K}(GABA)₈. Once dissolved, 34.17 g HBTU was added to the flask as a solid and allowed to dissolve. After 15 minutes of stirring, 23.0 mL of triethylamine (TEA) (0.211 mol, 2.2 eq) was added to the flask and the entire solution stirred at 25° C. under N₂ for 16 hours. An additional portion of DOHA (2.74 g), TEA (2.1 mL), and HBTU (5.70 g) was added after 16 hours and the mixture was stirred for an additional 2 hours. After overnight stirring, the solution was precipitated directly into 7:3 heptane/IPA. The product is redissolved in water and purified by tangential flow filtration. The aqueous solution is then freeze dried to yield the final product in 85% yield. ¹H NMR (500 MHz, D₂O): δ 6.65 (d, 1H), 6.57 (s, 1H), 6.50 (d, 1H), 4.09 (t, 2H), 3.15-3.75 (m, 226H), 2.94 (t, 2H), 2.63 (t, 2H), 2.32 (t, 2H), 1.90 (t, 2H), 1.43 (quint, 2H).

### Example 77

### Degradation Rates

Four samples according to the invention (Samples 77A-77D) were synthesized as follows, and a blend of two of the samples (Sample 77E) was also prepared. Each Sample was prepared and degradation studies were carried out as described. Degradation experiment was performed by independently preparing hydrogel samples from specified polymers and monitoring their weight loss over time in a solution of 2×PBS buffer at 37° C. Cured hydrogels were prepared generally by the methods described in Example 68. Specifically, 1.500 grams of polymer was loaded into a 10 mL luer lock syringe and sterilized. Separately 5 mL of 2×PBS buffer (pH=7.4) was loaded into a separate 5 mL luer lock syringe. The polymer was dissolved in the PBS buffer by a reciprocating motion using a female-female luer lock connection and kept in the 10 mL syringe. The polymer solution was dissolved. Separately, 5 mL of a 11.6 mg/mL solution of Sodium periodate in process water was placed in a 5 mL syringe. Both the polymer syringe and the periodate syringe were connected using a Micromedics blending “Y” connector and applicator. The solutions were expressed and the components mixed into a mold between glass plates. The mixture was cured for 10 minutes. After 10 minutes the hydrogel was removed and 10 mm diameter discs were punched out from the hydrogel sheet. Each cured polymer used in the study yielded fifteen discs. The discs were weighed to obtain an initial mass. Then, each disc was individually placed in a scintillation vial and filled with 15 mL of 2×PBS buffer. The vials were stored in an environmental chamber at 37° C. The pH was recorded weekly to ensure that a pH of 7.4 was maintained. If the pH of the solution fell outside of the range of 7.3 to 7.5, the solution was discarded and refilled with fresh 2×PBS buffer. At pre-determined time points, the vials were removed from the chamber, and the contents quantitatively transferred to pre-weighed 50 mL centrifuge tube. The scintillation vials were washed with 2 additional 15 mL portions of process water and transferred to the centrifuge tubes. the centrifuge tubes were then diluted to 50 mL with process water. The samples were spun, and the solutions were aspirated to remove the excess water. After 48±4 hours of vacuum drying at room temperature, The final mass of residual polymer hydrogel in each tube was recorded on an analytical balance.

Sample A: Synthesis of Medhesive-228(PEG10k-(β-Ala-FA)₄)

37.67 g of PEG_{10K}(β-Ala)₄ (prepared in a similar manner as in Example 47) was added to a 1 L round-bottom flask and dissolved in 260 mL of chloroform. once dissolved, 2.55 mL of diisopropylethylamine (DIEA) is added and allowed to stir. In a separate flask 4.27 g of ferulic acid is dissolved in 130 mL of chloroform, after which an additional 2.55 mL of DIEA is added. In a separate flask, 2.81 g of EDC.HCl is dissolved in 130 mL of chloroform. The EDC-chloroform solution is added to the ferulic acid-chloroform solution and stirred for two minutes at room temperature. At this point the resultant solution is added the round bottom flask containing PEG(β-Ala)₄ and stirred for 16 hours. An additional portion of ferulic acid (0.56 g), DIEA (0.51 mL), and EDC (0.56 g) was added after 16 hours and the mixture was stirred for an additional hour. The product mixture is concentrated and precipitated in a 70/30 mixture of Heptane/isopropyl alcohol. The product is redissolved in water and purified by tangential flow filtration. The aqueous solution is then freeze dried to yield the final product in 94% yield. ¹H NMR (500 MHz, D₂O): δ 7.35 (d, 1H), 7.17 (s, 1H), 7.07 (d, 1H), 6.85 (d, 1H), 6.40 (d, 1H), 4.19 (t, 2H), 3.8 (s, 3H), 3.25-3.75 (m, 228H, PEG and β-Ala resonances), 2.61 (t, 2H).

Sample B: Synthesis of Medhesive-229 (PEG10k-(GABA-FA)₄)

40.0 g of PEG_{10K}(GABA)₄ (prepared in a similar manner as in Example 46) was added to a 1 L round-bottom flask and dissolved in 275 mL of chloroform. once dissolved, 2.70 mL of diisopropylethylamine (DIEA) is added and allowed to stir. In a separate flask 4.51 g of ferulic acid is dissolved in 130 mL of chloroform, after which an additional 2.70 mL of DIEA is added. In a separate flask, 2.97 g of EDC.HCl is dissolved in 130 mL of chloroform. The EDC-chloroform solution is added to the ferulic acid-chloroform solution and stirred for two minutes at room temperature. At this point the resultant solution is added the round bottom flask containing PEG(GABA)₄ and stirred for 16 hours. An additional portion of ferulic acid (0.60 g), DIEA (0.54 mL), and EDC (0.59 g) was added after 16 hours and the mixture was stirred for an additional hour. The product mixture is concentrated and precipitated in a 70/30 mixture of Heptane/isopropyl alcohol. The product is redissolved in water and purified by tangential flow filtration. The aqueous solution is then freeze dried to yield the final product in 94% yield. ¹H NMR (500 MHz, D₂O): δ 7.35 (d, 1H), 7.16 (s, 1H), 7.07 (d, 1H), 6.85 (d, 1H), 6.40 (d, 1H), 4.16 (t, 2H), 3.80 (s, 3H), 3.30-3.75 (m, 226H, PEG resonances), 3.26 (t, 2H), 2.38 (t, 2H), 1.8 (t, 2H).

Sample C: Synthesis of Medhesive-230 (PEG1 Ok-(AVA-FA)₄)

40.0 g of PEG_{10K}(AVA)₄ (prepared in a similar manner as in Examples 46 and 47) was added to a 1 L round-bottom flask and dissolved in 275 mL of chloroform. once dissolved, 2.68 mL of diisopropylethylamine (DIEA) is added and allowed to stir. In a separate flask 4.49 g of ferulic acid is dissolved in 135 mL of chloroform, after which an additional 2.68 mL of DIEA is added. In a separate flask, 2.95 g of EDC.HCl is dissolved in 135 mL of chloroform. The EDC-chloroform solution is added to the ferulic acid-chloroform solution and stirred for two minutes at room temperature. At this point the resultant solution is added the round bottom flask containing PEG(GABA)₄ and stirred for 16 hours. An additional portion of ferulic acid (0.60 g), DIEA (0.54 mL), and EDC (0.59 g) was added after 16 hours and the mixture was stirred for an additional hour. The product mixture is concentrated and precipitated in a 70/30 mixture of Heptane/isopropyl alcohol. The product is redissolved in water and purified by tangential flow filtration. The aqueous solution is then freeze dried to yield the final product in 94% yield. ¹H NMR (500 MHz, D₂O): δ 7.35 (d, 1H), 7.16 (s, 1H), 7.07 (d, 1H), 6.85 (d, 1H), 6.40 (d, 1H), 4.17 (t, 2H), 3.81 (s, 3H), 3.25-3.75 (m, 226H, PEG resonances), 3.22 (t, 2H), 2.36 (t, 2H), 1.54 (m, 4H).

Sample D: Synthesis of Medhesive-235 (PEG10k-(β-Ala)₂(AVA-FA)₂)

37.57 g of PEG_{10K}[(β-Ala)₂(AVA)₂] (prepared in a similar manner as in Examples 46 and 47) was added to a 1 L round-bottom flask and dissolved in 258 mL of chloroform. once dissolved, 2.53 mL of diisopropylethylamine (DIEA) is added and allowed to stir. In a separate flask 4.24 g of ferulic acid is dissolved in 130 mL of chloroform, after which an additional 2.53 mL of DIEA is added. In a separate flask, 2.78 g of EDC.HCl is dissolved in 130 mL of chloroform. The EDC-chloroform solution is added to the ferulic acid-chloroform solution and stirred for two minutes at room temperature. At this point the resultant solution is added the round bottom flask containing PEG(GABA)₄ and stirred for 16 hours. An additional portion of ferulic acid (0.56 g), DIEA (0.50 mL), and EDC (0.55 g) was added after 16 hours and the mixture was stirred for an additional hour. The product mixture is concentrated and precipitated in a 70/30 mixture of Heptane/isopropyl alcohol. The product is redissolved in water and purified by tangential flow filtration. The aqueous solution is then freeze dried to yield the final product in 94% yield. ¹H NMR (500 MHz, D₂O): δ 7.37 (d, 1H), 7.17 (s, 1H), 7.07 (d, 1H), 6.85 (d, 1H), 6.42 (d, 1H), 4.20 (t, 2H)-β-Ala fragment, [4.17 (t, 2H)-AVA arms], 3.81 (s, 3H), 3.25-3.75 (m, 226H, PEG resonances), 3.22 (t, 2H), 2.60 (t, 2H), 2.36 (t, 2H), 1.54 (m, 4H).

Sample E: Blend of Medhesive-228 and Medhesive-230

10.00 g of Medhesive-228 and 10.00 grams of Medhesive-230 were combined, and dissolved in 500 mL of process water. The aqueous solution is then freeze dried and collected.

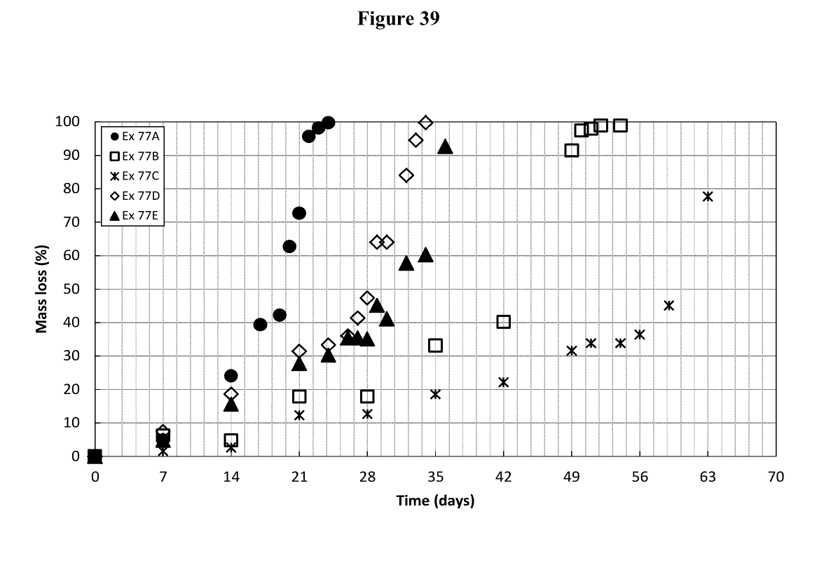

The degradation of these materials can be influenced in numerous ways through the use of specific linkers. Table 4, below, shows the degradation rates when the L group, L_{b}, Lₖ, Lₒ, Lᵣ has a linear alkyl spacer of 2, 3, or 4 carbons in length. Moreover, FIG. 39 is a graph with the degradation profiles for each of Example 77A-77E.

TABLE 4
In vitro Degradation data for Examples 77A-77E.

| | no. of carbons in the | | |
|---|---|---|---|
| | amino acid spacer for | % mass loss @ | days @ 20% |
| Example | L_{b}, Lₖ, Lₒ, Lᵣ | 21 days | mass loss |
| 77A | 2 | 72.6 | 13 |
| 77B | 3 | 17.8 | 27 |
| 77C | 4 | 12.3 | 38 |
| 77D | average = 3 | 31.4 | 15 |
| 77E | average = 3 | 27.7 | 17 |

Surprisingly, when these polymers are blended in various ratios a nonlinear effect may be achieved. For example, a 1:1 blend of two polymers where the first polymer (Example 77A), whose L_{b}, Lₖ, Lₒ, Lᵣ contains 2 carbons (e.g. L=β-Alanine) and a second polymer (Example 77C) whose L_{b}, Lₖ, Lₒ, Lᵣ contains 4 carbons (e.g. L=aminovaleric acid), degrades at a different rate than a polymer (Example 77B) whose L_{b}, Lₖ, Lₒ, Lᵣ contains 3 carbons (e.g. L=γ-aminobutyric acid). Additionally, when a single polymer (Example 77D) where 2 of the 4 linkers, L_{b}, Lₖ, Lₒ, Lᵣ, contain 2 carbons (e.g. β-Alanine), and the remaining 2 linkers, L_{b}, Lₖ, Lₒ, Lᵣ, contain 4 carbons (e.g. aminovaleric acid), also degrade at an even different rate than the single polymer (Example 77B) whose L_{b}, Lₖ, Lₒ, Lᵣ contains 3 carbons (e.g. L=γ-aminobutyric acid) or the aforementioned blend. Both approaches (i.e. multi-polymer blends or polymers with mixtures of L_{b}, Lₖ, Lₒ, Lᵣ) enable the fine tune tailoring of materials that degrade at a precise rate.

### REFERENCES

Najera et al., Recent synthetic uses of functionalized aromatic and heteroaromatic organolithium reagents prepared by non-deprotonating methods. Tetrahedron 59:2003:9255-9303
Malic et al., “Dye Comprising Functional Substituent”, WO2009/121148A1.
Xiao et al., “Photochromic Materials With Reactive Substituents”, U.S. Pat. No. 7,556,750B2.
Buchanan et al., “Aromatics Conversion With ITQ-13”, U.S. Pat. No. 7,081,556B2.
Kadoma et al., “A Comparative Study of the Radical-scavenging Activity of the Phenolcarboxylic Acids Caffeic Acid, p-Coumaric Acid, Chlorogenic Acid and Ferulic Acid, With or Without 2-Mercaptoethanol, a Thiol, Using the Induction Period Method.” Molecules; 2008: 2488-2499.
Trombino et al., Antioxidant Effect of Ferulic Acid in Isolated Membranes and Intact Cells: Synergistic Interactions with r-Tocopherol, â-Carotene, and Ascorbic Acid. J. Agric. Food Chem. 2004; 52:2411-2420.
Graf E. Antioxidant potential of ferulic acid. Free Radical BiologyMedicine. 13; 1992:435-448.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A compound comprising formula (I): 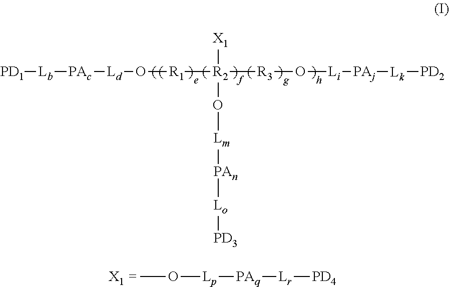
wherein
X₁ is optional;
each PD₁, PD₂, PD₃, and PD₄, independently, can be the same or different wherein each of PD₁, PD₂, PD₃, and PD₄, independently, is a residue of a formula comprising: 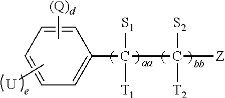
Wherein Q is a OH, SH, or NH₂
“d” is 1 to 5
U is a H, OH, OCH₃, O-PG, SH, S-PG, NH2, NH-PG, N(PG)₂, NO₂, F, Cl, Br, or I, or a combination thereof;
“e” is 1 to 5
“d+e” is equal to 5
each T₁ independently, is H, NH₂, OH, or COOH;
each S₁, independently, is H, NH₂, OH, or COOH;
each T₂, independently, is H, NH₂, OH, or COOH;
each S₂, independently, is H, NH₂, OH, or COOH;
Z is COOH, NH₂, OH or SH;
aa is a value of 0 to about 4;
bb is a value of 0 to about 4; and
Optionally, when one of the combinations of T₁ and T₂, S₁ and S₂, T₁ and S₂ or S₁ and T₂ are absent, then a double bond is formed between Cₐₐ and C_{bb}, and aa and bb are each at least 1 to form the double bond when present.
each L_{b}, Lₖ, Lₒ and Lᵣ, independently, can be the same or different;
optionally, each L_{d}, Lᵢ, Lₘ and Lₚ, if present, can be the same or different and if not present, represent a bond between the O and respective PA of the compound;
each PA_{c}, PAⱼ, PAₙ, and PA_{q}, independently, can be the same or different;
e is a value from 1 to about 3;
f is a value from 1 to about 10;
g is a value from 1 to about 3;
h is a value from 1 to about 10;
each of R₁, R₂ and R₃, independently, is a branched or unbranched alkyl group having at least 1 carbon atom;
each PA, independently, is a substantially poly(alkylene oxide) polyether or derivative thereof;
each L, independently, is a linker or is a suitable linking group selected from amide, ether, ester, urea, carbonate or urethane linking groups; and
each PD, independently, is a phenyl derivative.

2. The compound of claim 1, wherein each of PA_{c}, PAⱼ, PAₙ and PA_{q}, is a polyethylene glycol polyether or derivative thereof.

3. The compound of any of claims 1 through 2, wherein the molecular weight of each of the PAs is between about 1,500 and about 5,000 daltons.

4. The compound of any of claims 1 through 3, wherein each of L_{b}, Lₖ, Lₒ and Lᵣ are amide, ester, or a combination of amide and ester linkages and L_{d}, Lᵢ, Lₘ, and Lₚ represent ether bonds.

5. The compound of any of claims 1 through 4, wherein each R₁ and R₃ is a CH₂ and R₂ is a CH or CH₂—C—CH₂.

6. The compound of any of claims 1 through 5, wherein e and g each have a value of 1 and f has a value of 1 to 6.

7. The compound of any of claims 1 through 6, wherein h is 1 to 6.

8. The compound of claim 1, wherein X₁ is not present;
each of L_{b}, Lₖ, and Lₒ are amide linkages;
each of L_{d}, Lᵢ, and Lₘ represent ether bonds;
each of PA_{c}, PAⱼ, and PAₙ are polyethylene glycol polyether derivatives each comprising an amine terminal residue which form the amide linkages between the PD acid residue and the polyethylene glycol polyether derivative, each having a molecular weight of between about 1,500 and about 3,500 daltons;
wherein e, f and g each have a value of 1;
each R₁ and R₃ is a CH₂ and R₂ is a CH; and
h is 6.

9. The compound of claim 1, wherein X₁ is not present;
each of L_{b}, Lₖ, and Lₒ are a combination of amide and ester linkages;
each of L_{d}, Lᵢ, and Lₘ represent ether bonds;
each of PA_{c}, PAⱼ, and PAₙ are polyethylene glycol polyether derivatives each comprising a hydroxyl terminal residue having a molecular weight of between about 1,500 and about 3,500 daltons;
each L_{b}, Lₖ, and Lₒ represent an amino acid residue, where an ester bond is formed between the hydroxyl terminal of the polyethylene glycol polyether derivative and the carboxylic acid portion of the amino acid, and an amide bond is formed between the amine of the amino acid residue and the carboxylic acid portion of the PD
wherein e, f and g each have a value of 1;
each R₁ and R₃ is a CH₂ and R₂ is a CH; and
h is 6.

10. The compound of claim 1, wherein X₁ is not present;
each of L_{b}, Lₖ, and Lₒ are a combination of amide and ester linkages;
each of L_{d}, Lᵢ, and Lₘ represent ether bonds;
each of PA_{c}, PAⱼ, and PAₙ are polyethylene glycol polyether derivatives each comprising a hydroxyl terminal residue having a molecular weight of between about 1,500 and about 3,500 daltons;
each L_{b}, Lₖ, and Lₒ represent a dicarboxylic acid residue, where an ester bond is formed between the hydroxyl terminal of the polyethylene glycol polyether derivative and one terminal portion of the dicarboxylic acid, and an amide bond is formed between the second terminal portion of the dicarboxylic acid residue and the terminal amine portion of the PD
wherein e, f and g each have a value of 1;
each R₁ and R₃ is a CH₂ and R₂ is a CH; and
h is 6.

11. The compound of claim 1, wherein X₁ is not present;
each of L_{b}, Lₖ, and Lₒ are urethane linkages between the terminal amine residue of the PD and the terminal portion of the polyethylene glycol polyether;
each of L_{d}, Lᵢ, and Lₘ represent ether bonds;
each of PA_{c}, PAⱼ, and PAₙ are polyethylene glycol polyether derivatives each comprising a hydroxyl terminal residue having a molecular weight of between about 1,500 and about 3,500 daltons;
wherein e, f and g each have a value of 1;
each R₁ and R₃ is a CH₂ and R₂ is a CH; and
h is 6.

12. The compound of claim 1, wherein X₁ is not present;
each of L_{b}, Lₖ, and Lₒ are urea linkages between the terminal amine residue of the PD and the terminal portion of the polyethylene glycol polyether;
each of L_{d}, Lᵢ, and Lₘ represent ether bonds;
each of PA_{c}, PAⱼ, and PAₙ are polyethylene glycol polyether derivatives each comprising an amine terminal residue having a molecular weight of between about 1,500 and about 3,500 daltons;
wherein e, f and g each have a value of 1;
each R₁ and R₃ is a CH₂ and R₂ is a CH; and
h is 6.

13. The compound of claim 1, wherein X₁ is present;
each of L_{b}, Lₖ, Lₒ, and Lᵣ are amide linkages;
each of L_{d}, Lᵢ, Lₘ, and Lₚ represent ether bonds;
each of PA_{c}, PAⱼ, PAₙ, and PA_{q} are polyethylene glycol polyether derivatives each comprising an amine terminal residue which form the amide linkages between the PD acid residue and the polyethylene glycol polyether derivative, each having a molecular weight of between about 1,500 and about 3,500 daltons;
wherein e, f and g each have a value of 1;
each R₁ and R₃ is a CH₂ and R₂ is a CH₂—C—CH₂; and
h is 1.

14. The compound of claim 1, wherein X₁ is present;
each of L_{b}, Lₖ, Lₒ, and Lᵣ are a combination of amide and ester linkages;
each of L_{d}, Lᵢ, Lₘ, L represent ether bonds;
each of PA_{c}, PAⱼ, PAₙ, and PA_{q} are polyethylene glycol polyether derivatives each comprising a hydroxyl terminal residue having a molecular weight of between about 1,500 and about 3,500 daltons;
each L_{b}, Lₖ, Lₒ, and Lᵣ represent an amino acid residue, where an ester bond is formed between the hydroxyl terminal of the polyethylene glycol polyether derivative and the carboxylic acid portion of the amino acid, and an amide bond is formed between the amine of the amino acid residue and the carboxylic acid portion of the PD
wherein e, f and g each have a value of 1;
each R₁ and R₃ is a CH₂ and R₂ is a CH₂—C—CH₂; and
h is 1.

15. The compound of claim 1, wherein X₁ is present;
each of L_{b}, Lₖ, Lₒ, and Lᵣ are a combination of amide and ester linkages;
each of L_{d}, Lᵢ, Lₘ and Lₚ represent ether bonds;
each of PA_{c}, PAⱼ, PAₙ, and PA_{q} are polyethylene glycol polyether derivatives each comprising a hydroxyl terminal residue having a molecular weight of between about 1,500 and about 3,500 daltons;
each L_{b}, Lₖ, Lₒ, and Lᵣ represent a dicarboxylic acid residue, where an ester bond is formed between the hydroxyl terminal of the polyethylene glycol polyether derivative and one terminal portion of the dicarboxylic acid, and an amide bond is formed between the second terminal portion of the dicarboxylic acid residue and the terminal amine portion of the PD
wherein e, f and g each have a value of 1;
each R₁ and R₃ is a CH₂ and R₂ is a CH₂—C—CH₂; and
h is 1.

16. The compound of claim 1, wherein X₁ is present;
each of L_{b}, Lₖ, Lₒ, and Lᵣ are urethane linkages between the terminal amine residue of the PD and the terminal portion of the polyethylene glycol polyether;
each of L_{d}, Lᵢ, Lₘ, and Lₚ represent ether bonds;
each of PA_{c}, PAⱼ, PAₙ, and PA_{q} are polyethylene glycol polyether derivatives each comprising a hydroxyl terminal residue having a molecular weight of between about 1,500 and about 3,500 daltons;
wherein e, f and g each have a value of 1;
each R₁ and R₃ is a CH₂ and R₂ is a CH₂—C—CH₂; and
h is 1.

17. The compound of claim 1, wherein X₁ is present;
each of L_{b}, Lₖ, Lₒ, and Lᵣ are urea linkages between the terminal amine residue of the PD and the terminal portion of the polyethylene glycol polyether;
each of L_{d}, Lᵢ, Lₘ, and Lₚ represent ether bonds;
each of PA_{c}, PAⱼ, PAₙ, and PA_{q} are polyethylene glycol polyether derivatives each comprising an amine terminal residue having a molecular weight of between about 1,500 and about 3,500 daltons;
wherein e, f and g each have a value of 1;
each R₁ and R₃ is a CH₂ and R₂ is a CH₂—C—CH₂; and
h is 1.

18. The compound of claim 1, wherein X₁ is present;
each of L_{b}, Lₖ, Lₒ, and Lᵣ are amide linkages;
each of L_{d}, Lᵢ, Lₘ, and Lₚ represent ether bonds;
each of PA_{c}, PAⱼ, PAₙ, and PA_{q} are polyethylene glycol polyether derivatives each comprising an amine terminal residue which form the amide linkages between the PD acid residue and the polyethylene glycol polyether derivative, each having a molecular weight of between about 1,500 and about 3,500 daltons;
wherein e, f and g each have a value of 1;
each R₁ and R₃ is a CH₂ and R₂ is a CH₂—C—CH₂; and
h is 2.

19. The compound of claim 1, wherein X₁ is present;
each of L_{b}, Lₖ, Lₒ, and Lᵣ are a combination of amide and ester linkages;
each of L_{d}, Lᵢ, Lₘ, L represent ether bonds;
each of PA_{c}, PAⱼ, PAₙ, and PA_{q} are polyethylene glycol polyether derivatives each comprising a hydroxyl terminal residue having a molecular weight of between about 1,500 and about 3,500 daltons;
each L_{b}, Lₖ, Lₒ, and Lᵣ represent an amino acid residue, where an ester bond is formed between the hydroxyl terminal of the polyethylene glycol polyether derivative and the carboxylic acid portion of the amino acid, and an amide bond is formed between the amine of the amino acid residue and the carboxylic acid portion of the PD
wherein e, f and g each have a value of 1;
each R₁ and R₃ is a CH₂ and R₂ is a CH₂—C—CH₂; and
h is 2.

20. The compound of claim 1, wherein X₁ is present;
each of L_{b}, Lₖ, Lₒ, and Lᵣ are a combination of amide and ester linkages;
each of L_{d}, Lᵢ, Lₘ and Lₚ represent ether bonds;
each of PA_{c}, PAⱼ, PAₙ, and PA_{q} are polyethylene glycol polyether derivatives each comprising a hydroxyl terminal residue having a molecular weight of between about 1,500 and about 3,500 daltons;
each L_{b}, Lₖ, Lₒ, and Lᵣ represent a dicarboxylic acid residue, where an ester bond is formed between the hydroxyl terminal of the polyethylene glycol polyether derivative and one terminal portion of the dicarboxylic acid, and an amide bond is formed between the second terminal portion of the dicarboxylic acid residue and the terminal amine portion of the PD
wherein e, f and g each have a value of 1;
each R₁ and R₃ is a CH₂ and R₂ is a CH₂—C—CH₂; and
h is 2.

21. The compound of claim 1, wherein X₁ is present;
each of L_{b}, Lₖ, Lₒ, and Lᵣ are urethane linkages between the terminal amine residue of the PD and the terminal portion of the polyethylene glycol polyether;
each of L_{d}, Lᵢ, Lₘ, and Lₚ represent ether bonds;
each of PA_{c}, PAⱼ, PAₙ, and PA_{q} are polyethylene glycol polyether derivatives each comprising a hydroxyl terminal residue having a molecular weight of between about 1,500 and about 3,500 daltons;
wherein e, f and g each have a value of 1;
each R₁ and R₃ is a CH₂ and R₂ is a CH₂—C—CH₂; and
h is 2.

22. The compound of claim 1, wherein X₁ is present;
each of L_{b}, Lₖ, Lₒ, and Lᵣ are urea linkages between the terminal amine residue of the PD and the terminal portion of the polyethylene glycol polyether;
each of L_{d}, Lᵢ, Lₘ, and Lₚ represent ether bonds;
each of PA_{c}, PAⱼ, PAₙ, and PA_{q} are polyethylene glycol polyether derivatives each comprising an amine terminal residue having a molecular weight of between about 1,500 and about 3,500 daltons;
wherein e, f and g each have a value of 1;
each R₁ and R₃ is a CH₂ and R₂ is a CH₂—C—CH₂; and
h is 2.

23. The compound of claim 1, wherein X₁ is present;
each of L_{b}, Lₖ, Lₒ, and Lᵣ are amide linkages;
each of L_{d}, Lᵢ, Lₘ, and Lₚ represent ether bonds;
each of PA_{c}, PAⱼ, PAₙ, and PA_{q} are polyethylene glycol polyether derivatives each comprising an amine terminal residue which form the amide linkages between the PD acid residue and the polyethylene glycol polyether derivative, each having a molecular weight of between about 1,500 and about 3,500 daltons;
wherein e, f and g each have a value of 1;
each R₁ and R₃ is a CH₂ and R₂ is a CH₂—C—CH₂; and
h is 3.

24. The compound of claim 1, wherein X₁ is present;
each of L_{b}, Lₖ, Lₒ, and Lᵣ are a combination of amide and ester linkages;
each of L_{d}, Lᵢ, Lₘ, L represent ether bonds;
each of PA_{c}, PAⱼ, PAₙ, and PA_{q} are polyethylene glycol polyether derivatives each comprising a hydroxyl terminal residue having a molecular weight of between about 1,500 and about 3,500 daltons;
each L_{b}, Lₖ, Lₒ, and Lᵣ represent an amino acid residue, where an ester bond is formed between the hydroxyl terminal of the polyethylene glycol polyether derivative and the carboxylic acid portion of the amino acid, and an amide bond is formed between the amine of the amino acid residue and the carboxylic acid portion of the PD
wherein e, f and g each have a value of 1;
each R₁ and R₃ is a CH₂ and R₂ is a CH₂—C—CH₂; and
h is 3.

25. The compound of claim 1, wherein X₁ is present;
each of L_{b}, Lₖ, Lₒ, and Lᵣ are a combination of amide and ester linkages;
each of L_{d}, Lᵢ, Lₘ and Lₚ represent ether bonds;
each of PA_{c}, PAⱼ, PAₙ, and PA_{q} are polyethylene glycol polyether derivatives each comprising a hydroxyl terminal residue having a molecular weight of between about 1,500 and about 3,500 daltons;
each L_{b}, Lₖ, Lₒ, and Lᵣ represent a dicarboxylic acid residue, where an ester bond is formed between the hydroxyl terminal of the polyethylene glycol polyether derivative and one terminal portion of the dicarboxylic acid, and an amide bond is formed between the second terminal portion of the dicarboxylic acid residue and the terminal amine portion of the PD
wherein e, f and g each have a value of 1;
each R₁ and R₃ is a CH₂ and R₂ is a CH₂—C—CH₂; and
h is 3.

26. The compound of claim 1, wherein X₁ is present;
each of L_{b}, Lₖ, Lₒ, and Lᵣ are urethane linkages between the terminal amine residue of the PD and the terminal portion of the polyethylene glycol polyether;
each of L_{d}, Lᵢ, Lₘ, and Lₚ represent ether bonds;
each of PA_{c}, PAⱼ, PAₙ, and PA_{q} are polyethylene glycol polyether derivatives each comprising a hydroxyl terminal residue having a molecular weight of between about 1,500 and about 3,500 daltons;
wherein e, f and g each have a value of 1;
each R₁ and R₃ is a CH₂ and R₂ is a CH₂—C—CH₂; and
h is 3.

27. The compound of claim 1, wherein X₁ is present;
each of L_{b}, Lₖ, Lₒ, and Lᵣ are urea linkages between the terminal amine residue of the PD and the terminal portion of the polyethylene glycol polyether;
each of L_{d}, Lᵢ, Lₘ, and Lₚ represent ether bonds;
each of PA_{c}, PAⱼ, PAₙ, and PA_{q} are polyethylene glycol polyether derivatives each comprising an amine terminal residue having a molecular weight of between about 1,500 and about 3,500 daltons;
wherein e, f and g each have a value of 1;
each R₁ and R₃ is a CH₂ and R₂ is a CH₂—C—CH₂; and
h is 3.

28. A compound of any of claims 1 through 27, with an oxidant

29. A blend of a polymer and a compound of any of claims 1 through 28.

30. The blend of claim 29, wherein the polymer is present in a range of about 1 to about 50 percent by weight.

31. The blend of claim 30, wherein the polymer is present in a range of about 1 to about 30 percent by weight.

32. A blend of a polymer and a compound of any of claims 29 through 31 with an oxidant.

33. A blend of a first compound of claim 1, where L_{b}, Lₖ, Lₒ, and Lᵣ each comprise 2 carbons, and a second compound of claim 1, where L_{b}, Lₖ, Lₒ, and Lᵣ each comprise 4 carbons.

34. The blend of claim 33, wherein the first compound and the second compound are provided in a 1:1 weight ratio.

35. A bioadhesive construct comprising:
a support suitable for tissue repair or reconstruction; and
a coating comprising any of the blends of claims 29 through 31.

36. The bioadhesive construct of claim 35, further comprising an oxidant.

37. The bioadhesive construct of either of claims 30, 31 or 35, wherein the oxidant is formulated with the coating.

38. The bioadhesive construct of either of claims 30, 31 or 35, wherein the oxidant is applied to the coating.

39. The bioadhesive construct of any of claims 30, 31, or 35 through 38, wherein the support is a film, a mesh, a membrane, a nonwoven or a prosthetic.

40. A bioadhesive construct comprising:
a support suitable for tissue repair or reconstruction;
a first coating comprising a phenyl derivative (PD) functionalized polymer (PDp) of any of claims 1 through 27 and a polymer; and
a second coating coated onto the first coating, wherein the second coating comprises a phenyl derivative (PD) functionalized polymer (PDp) of any of claims 1 through 27.

41. A bioadhesive construct comprising:
a support suitable for tissue repair or reconstruction;
a first coating comprising a first phenyl derivative (PD) functionalized polymer (PDp) of any of claims 1 through 27 and a first polymer; and
a second coating coated onto the first coating, wherein the second coating comprises a second phenyl derivative (PD) functionalized polymer (PDp) of any of claims 1 through 27 and a second polymer, wherein the first and second polymer may be the same or different and wherein the first and second PDp can be the same or different.

42. A bioadhesive construct comprising:
a support suitable for tissue repair or reconstruction;
a first coating comprising a first phenyl derivative (PD) functionalized polymer (PDp) of any of claims 1 through 27; and
a second coating coated onto the first coating, wherein the second coating comprises a second phenyl derivative (PD) functionalized polymer (PDp) of any of claims 1 through 27, wherein the first and second PDp can be the same or different.

43. A bioadhesive construct of any of claims 40 through 42 formulated with oxidant.

44. A method to reduce bacterial growth on a substrate surface, comprising the step of coating a phenyl derivative (PD) functionalized polymer (PDp) of any of claims 1 through 27 onto the surface of the substrate.

45. The compound of claim 1, wherein at least 1 of the linkers L_{b}, Lₖ, Lₒ, Lᵣ, L_{d}, Lᵢ, Lₘ, and Lₚ is different from at least one other of said linkers.
